SEQUENCE LISTING (20 sequences)

SEQ ID NO: 1 - DNA, 1139 bp, Homo sapiens
gagcggatgttcacttcttctccacaatgaatgagtgtcactatgacaagcacatggactttttttataataggagcaacactgatactgtcgatgactggacaggaacaaagcttgtgattgttttgtgtgttgggacgtttttctgcctgtttatttttttttctaattctctggtcatcgcggcagtgatcaaaaacagaaaatttcatttccccttctactacctgttggctaatttagctgctgccgatttcttcgctggaattgcctatgtattcctgatgtttaacacaggcccagtttcaaaaactttgactgtcaaccgctggtttctccgtcaggggcttctggacagtagcttgactgcttccctcaccaacttgctggttatcgccgtggagaggcacatgtcaatcatgaggatgcgggtccatagcaacctgaccaaaaagagggtgacactgctcattttgcttgtctgggccatcgccatttttatgggggcggtccccacactgggctggaattgcctctgcaacatctctgcctgctcttccctggcccccatttacagcaggagttaccttgttttctggacagtgtccaacctcatggccttcctcatcatggttgtggtgtacctgcggatctacgtgtacgtcaagaggaaaaccaacgtcttgtctccgcatacaagtgggtccatcagccgccggaggacacccatgaagctaatgaagacggtgatgactgtcttaggggcgtttgtggtatgctggaccccgggcctggtggttctgctcctcgacggcctgaactgcaggcagtgtggcgtgcagcatgtgaaaaggtggttcctgctgctggcgctgctcaactccgtcgtgaaccccatcatctactcctacaaggacgaggacatgtatggcaccatgaagaagatgatctgctgcttctctcaggagaacccagagaggcgtccctctcgcatcccctccacagtcctcagcaggagtgacacaggcagccagtacatagaggatagtattagccaaggtgcagtctgcaataaaagcacttcctaaactctggatgcctctcggcccacccaggcctcctctgggaaaagagctgtt

SEQ ID NO: 2 - protein, 353 aa, Homo sapiens
MNECHYDKHMDFFYNRSNTDTVDDWTGTKLVIVLCVGTFFCLFIFFSNSLVIAAVIKNRKFHFPFYYLLANLAAADFFAGIAYVFLMFNTGPVSKTLTVNRWFLRQGLLDSSLTASLTNLLVIAVERHMSIMRMRVHSNLTKKRVTLLILLVWAIAIFMGAVPTLGWNCLCNISACSSLAPIYSRSYLVFWTVSNLMAFLIMVVVYLRIYVYVKRKTNVLSPHTSGSISRRRTPMKLMKTVMTVLGAFVVCWTPGLVVLLLDGLNCRQCGVQHVKRWFLLLALLNSVVNPIIYSYKDEDMYGTMKKMICCFSQENPERRPSRIPSTVLSRSDTGSQYIEDSISQGAVCNKSTS

SEQ ID NO: 3 - DNA, 23 bp, Artificial Sequence
  n = inosine
gyntwyrynntnwsntgghtncc

SEQ ID NO: 4 - DNA, 23 bp, Artificial Sequence
  n = inosine
avnadngbrwavannannggrtt

SEQ ID NO: 5 - DNA, 25 bp, Artificial Sequence
  Description of Artificial Sequence primer
ttatgcttccggctcgtatgttgtg

SEQ ID NO: 6 - DNA, 26 bp, Artificial Sequence
  Description of Artificial Sequence primer
atgtgctgcaaggcgattaagttggg

SEQ ID NO: 7 - DNA, 35 bp, Artificial Sequence
  Description of Artificial Sequence primer
atctatctcgagcctgggtgggccgagaggcatcc

SEQ ID NO: 8 - DNA, 24 bp, Artificial Sequence
  Description of Artificial Sequence primer
gcaggcagtgtggcgtgcagcatg

SEQ ID NO: 9 - DNA, 24 bp, Artificial Sequence
  Description of Artificial Sequence primer
tctgctcctcgacggcctgaactg

SEQ ID NO: 10 - DNA, 48 bp, Artificial Sequence
  Description of Artificial Sequence primer
gtgaaaaggtggttcctgctgctggcgctgctcaactccgtcgtgaac

SEQ ID NO: 11 - DNA, 34 bp, Artificial Sequence
  Description of Artificial Sequence primer
gtcacgaattcgtcatgacactgctcattttgct

SEQ ID NO: 12 - DNA, 34 bp, Artificial Sequence
  Description of Artificial Sequence primer
gtcacggatccgtcatgtcatcaccgtcttcatt

SEQ ID NO: 13 - DNA, 19 bp, Artificial Sequence
  Description of Artificial Sequence primer
tctctgcctgctcttccct

SEQ ID NO: 14 - DNA, 18 bp, Artificial Sequence
  Description of Artificial Sequence primer
cataccacaaacgcccct

SEQ ID NO: 15 - protein, 364 aa, Homo sapiens
MAAISTSIPVISQPQFTAMNEPQCFYNESIAFFYNRSGKHLATEWNTVSKLVMGLGITVCIFIMLANLLVMVAIYVNRRFHFPIYYLMANLAAADFFAGLAYFYLMFNTGPNTRRLTVSTWLLRQGLIDTSLTASVANLLAIAIERHITVFRMQLHTRMSNRRVVVVIVVIWTMAIVMGAIPSVGWNCICDIENCSNMAPLYSDSYLVFWAIFNLVTFVVMVVLYAHIFGYVRQRTMRMSRHSSGPRRNRDTMMSLLKTVVIVLGAFIICWTPGLVLLLLDVCCPQCDVLAYEKFFLLLAEFNSAMNPIIYSYRDKEMSATFRQILCCQRSENPTGPTEGSDRSASSLNHTILAGVHSNDHSVV

SEQ ID NO: 16 - protein, 382 aa, Homo sapiens
MVIMGQCYYNETIGFFYNNSGKELSSHWRPKDVVVVALGLTVSVLVLLTNLLVIAAIASNRRFHQPIYYLLGNLAAADLFAGVAYLFLMFHTGPRTARLSLEGWFLRQGLLDTSLTASVATLLAIAVERHRSVMAVQLHSRLPRGRVVMLIVGVWVAALGLGLLPAHSWHCLCALDRCSRMAPLLSRSYLAVWALSSLLVFLLMVAVYTRIFFYVRRRVQRMAEHVSCHPRYRETTLSLVKTVVIILGAFVVCWTPGQVVLLLDGLGCESCNVLAVEKYFLLLAEANSLVNAAVYSCRDAEMRRTFRRLLCCACLRQSTRESVHYTSSAQGGASTRIMLPENGHPLMTPPFSYLELQRYAASNKSTAPDDLWVLLAQPNQQD

SEQ ID NO: 17 - protein, 381 aa, Homo sapiens
MGPTSVPLVKAHRSSVSDYVNYDIIVRHYNYTGKLNISADKENSIKLTSVVFILICCFIILENIFVLLTIWKTKKFHRPMYYFIGNLALSDLLAGVAYTANLLLSGATTYKLTPAQWFLREGSMFVALSASVFSLLAIAIERYITMLKMKLHNGSNNFRLFLLISACWVISLILGGLPIMGWNCISALSSCSTVLPLYHKHYILFCTTVFTLLLLSIVILYCRIYSLVRTRSRRLTFRKNISKASRSSENVALLKTVIIVLSVFIACWAPLFILLLLDVGCKVKTCDILFRAEYFLVLAVLNSGTNPIIYTLTNKEMRRAFIRIMSCCKCPSGDSAGKFKRPIIAGMEFSRSKSDNSSHPQKDEGDNPETIMSSGNVNSSS

SEQ ID NO: 18 - protein, 378 aa, Homo sapiens
MATALPPRLQPVRGNETLREHYQYVGKLAGRLKEASEGSTLTTVLFLVICSFIVLENLMVLIAIWKNNKFHNRMYFFIGNLALCDLLAGIAYKVNILMSGKKTFSLSPTVWFLREGSMFVALGASTCSLLAIAIERHLTMIKMRPYDANKRHRVFLLIGMCWLIAFTLGALPILGWNCLHNLPDCSTILPLYSKKYIAFCISIFTAILVTIVILYARIYFLVKSSSRKVANHNNSERSMALLRTVVIVVSVFIACWSPLFILFLIDVACRVQACPILFKAQWFIVLAVLNSAMNPVIYTLASKEMRRAFFRLVCNCLVRGRGARASPIQPALDPSRSKSSSSNNSSHSPKVKEDLPHTDPSSCIMDKNAALQNGIFCN

SEQ ID NO: 19 - protein, 353 aa, Homo sapiens
MGSLYSEYLNPNKVQEHYNYTKETLETQETTSRQVASAFIVILCCAIVVENLLVLIAVARNSKFHSAMYLFLGNLAASDLLAGVAFVANTLLSGSVTLRLTPVQWFAREGSASITLSASVFSLLAIAIERHVAIAKVKLYGSDKSCRMLLLIGASWLISLVLGGLPILGWNCLGHLEACSTVLPLYAKHYVLCVVTIFSIILLAIVALYVRIYCVVRSSHADMAAPQTLALLKTVTIVLGVFIVCWLPAFSILLLDYACPVHSCPILYKAHYFFAVSTLNSLLNPVIYTWRSRDLRREVLRPLQCWRPGVGVQGRRRVGTPGHHLLPLRSSSSLERGMHMPTSPTFLEGNTVV

SEQ ID NO: 20 - protein, 383 aa, Homo sapiens
MNATGTPVAPESCQQLAAGGHSRLIVLHYNHSGRLAGRGGPEDGGLGALRGLSVAASCLVVLENLLVLAAITSHMRSRRWVYYCLVNITLSDLLTGAAYLANVLLSGARTFRLAPAQWFLREGLLFTALAASTFSLLFTAGERFATMVRPVAESGATKTSRVYGFIGLCWLLAALLGMLPLLGWNCLCAFDRCSSLLPLYSKRYILFCLVIFAGVLATIMGLYGAIFRLVQASGQKAPRPAARRKARRLLKTVLMILLAFLVCWGPLFGLLLADVFGSNLWAQEYLRGMDWILALVLNSAVNPIIYSFRSREVCRAVLSFLCCGCLRLGMRGPGDCLARAVEAHSGASTTDSSLRPRDSGRFSRSLSFRMREPLSSISSVRSI

(19) United States
(12) Patent Application Publication
(10) Pub. No.: US 2003/0130493 A1
(43) Pub. Date: Jul. 10, 2003

(54) DNA encoding Edg7 receptor

(76) Inventors: James A. Bonini, Oakland, NJ (US); Ling Yan Huang, Kearny, NJ (US); Beth E. Borowsky, Flemington, NJ (US); John A. Salon, Santa Paula, CA (US); Amy Wilson, Woodstock, NY (US); Raisa Nagorny, Fair Lawn, NJ (US)

Correspondence Address: John P. White, Cooper & Dunham LLP, 1185 Avenue of the Americas, New York, NY, 10036, US

(21) Appl. No.: 10/228,762

(22) Filed: Aug. 26, 2002

Related U.S. Application Data
(63) Continuation-in-part of application No. 09/356,315, filed on Jul. 16, 1999, now abandoned.
Application No. 09/356,315 is a continuation-in-part of application No. 09/253,998, filed on Feb. 22, 1999, now abandoned.

(51) Int. Cl. C07H 21/04; C12Q 1/68; C07K 14/705; C12P 21/02; C12N 5/06; A61K 38/17

(52) U.S. Cl. 536/23.5; 530/350; 514/12; 435/6; 435/69.1; 435/320.1; 435/325

(57) ABSTRACT

This invention provides isolated nucleic acids encoding mammalian Edg7 receptors, purified mammalian Edg7 receptors, vectors comprising nucleic acid encoding mammalian Edg7 receptors, cells comprising such vectors, antibodies directed to mammalian Edg7 receptors, nucleic acid probes useful for detecting nucleic acid encoding mammalian Edg7 receptors, antisense oligonucleotides complementary to unique sequences of nucleic acid encoding mammalian Edg7 receptors, transgenic, nonhuman animals which express DNA encoding normal or mutant mammalian Edg7 receptors, methods of isolating mammalian Edg7 receptors, methods of treating an abnormality that is linked to the activity of the mammalian Edg7 receptors, as well as methods of determining binding of compounds to mammalian Edg7 receptors, methods of identifying agonists and antagonists of Edg7 receptors, and agonists and antagonists so identified.

### BACKGROUND OF THE INVENTION

This application is a continuation-in-part of U.S. Ser. No. 09/253,998, filed Feb. 22, 1999, the contents of which are hereby incorporated by reference into the subject application.

Throughout this application various publications are referred to by author(s) and year within parenthesis. Full citations for these publications may be found at the end of the specification immediately preceding the claims. The disclosures of these publications, in their entireties, are hereby incorporated by reference into this application in order to more fully describe the state of the art to which the invention pertains.

Neuroregulators comprise a diverse group of natural products that subserve or modulate communication in the nervous system. They include, but are not limited to, neuropeptides, amino acids, biogenic amines, lipids and lipid metabolites, and other metabolic byproducts. Many of these neuroregulator substances interact with specific cell surface receptors which transduce signals from the outside to the inside of the cell. G-protein coupled receptors (GPCRs) represent a major class of cell surface receptors with which many neurotransmitters interact to mediate their effects. GPCRs are characterized by seven membrane-spanning domains and are coupled to their effectors via G-proteins linking receptor activation with intracellular biochemical sequelae such as stimulation of adenylyl cyclase.

Phospholipids represent an emerging class of endogenous activators of GPCRs (Moolenaar et al., 1997; Goetzl and An, 1998; Nietgen and Durieux, 1998). Lysophosphatidic acid (LPA) is structurally the simplest glycerophospholipid and consists of a glycerol backbone with an acyl group at the sn-1 position, a hydroxyl group at the sn-2 position, and a phosphate group at the sn-3 position. It can be generated through the hydrolysis of pre-existing phospholipids. One of the best characterized pathways for LPA production is deacylation of phosphatidic acid by the enzyme phospholipase A₂ (Fourcade et al., 1995), although other biosynthetic pathways may contribute to LPA generation (Gaits et al., 1997a). LPA has been found to be present in various fluids associated with pathophysiological conditions. In serum LPA is produced by thrombin-activated platelets and is present in the micromolar range (Jalink et al., 1994). Also ascites fluid from ovarian cancer patients (Westermann et al., 1998; Xu et al., 1998), and cerebrospinal fluid of piglets injected with hematoma blood (Tigyi et al., 1995; Yakubu et al., 1997) contain LPA. In addition, growth factor-stimulated fibroblasts (Jalink et al., 1994) and alpha₂ adrenergic receptor-stimulated adipocytes (Valet et al., 1998) can produce LPA.

Historically, LPA was believed to be just an intermediary in phospholipid metabolism; however, recently it has gained recognition as an intercellular lipid mediator with diverse effects in a wide variety of cells and tissues (Jalink et al., 1994; Moolenaar, 1995). LPA-mediated cellular responses range from modulation of signaling pathways such as reduction in cAMP, to cell growth-related effects such as DNA transcription and mitogenesis, to cytoskeleton reorganization-related responses such as stress fiber formation and neurite retraction. Accumulating evidence suggests that LPA produces these diverse effects by activating one or more surface GPCRs. The LPA-induced hydrolysis of phosphoinositides and reduction in cAMP accumulation are sensitive to guanine nucleotides and pertussis toxin (PTX), respectively (Plevin et al., 1991). In addition, photoaffinity labeling of a putative 38 kDa membrane receptor by [³²P]diazirine-LPA was observed in rat brain membranes and in various cell lines (van der Bend et al., 1992). Similarly, Thomson et al. (1994) observed specific binding of [³H]LPA in the rat brain and Swiss 3T3 membranes with affinities (K_{d} values) of 2 and 5nM and maximum number of binding sites (B_{Max}) of 19 and 38 fmol/μg protein, respectively. This LPA binding was sensitive to guanine nucleotides, suggesting that the binding site may represent a GPCR. The recent cloning of several heptahelical GPCRs which preferentially respond to LPA, as compared to other phospholipids, supports the idea that LPA indeed produces its effects via activation of GPCRs.

To date, three GPCRs, for which LPA is proposed to be an endogenous ligand, have been cloned. Two of these receptors belong to a new class of GPCRs called the Endothelial Differentiation Gene (Edg) family. The Edg receptor family, six members of which have been cloned thus far, can be subdivided into two groups on the basis of amino acid homology and ligand selectivity (Goetzl and An, 1998). Each of the Edg1, Edg3 and Edg5 receptor demonstrates >40% amino acid identity with each of the others and is preferentially activated by a sphingophospholipid, sphingosine-1-phosphate (SiP) (An et al., 1997b; Lee et al., 1998). In contrast, LPA is the preferred agonist for Edg2 and Edg4 receptors which exhibit >40% amino acid sequence identity between them. The amino acid identity between S1P-activated and LPA-activated cloned Edg receptors is 30-33% (Goetzl and An, 1998). Edg6, a recently cloned receptor, exhibits 37-46% identity to other Edg family members; however, its endogenous ligand is not yet known (Graler et al., 1998).

Edg2 (also called ventricular zone gene-1 or vzg-1) was the first mammalian LPA receptor to be cloned whose protein expression was developmentally regulated (Hecht et al., 1996). Edg2 overexpression in cortical neuroblastoma cells resulted in decreases in EC₅₀ values for LPA-induced cell rounding and reduction in cAMP production. However, other structurally related lysophospholipids, such as lysophosphatidylethanolamine, lysophosphatidylglycerol, and lysophosphatidylcholine were unable to enhance cell rounding in Edg2-expressing cells, demonstrating specificity of the response to LPA and implying that Edg2 was indeed an LPA receptor. Similar results were obtained with the human homologue of Edg2, overexpression of which resulted in enhancement of [³H]LPA binding and LPA-induced transcription in a serum response element (SRE) reporter gene assay (An et al., 1997a), in LPA-mediated calcium mobilization (An et al., 1998b), and in activation of yeast pheromone response pathway (Erickson et al., 1998). Heterologous expression of Edg4, cloned on the basis of its sequence similarity to Edg2, results in biological responses very similar to those observed with Edg2, namely, an increase in specific binding of [³H]LPA, induction of gene transcription and calcium mobilization (An et al., 1998a; An et al., 1998b). Even though Edg2 and Edg4 receptors seem to have similar biological endpoints, their tissue distribution is unique. Edg2 is expressed more widely in many central and peripheral tissues such as brain, heart, kidney, pancreas, prostate and small intestine (Hecht et al., 1996; An et al., 1997a). In contrast, the expression of Edg4 is restricted to peripheral tissues such as thymus, spleen, testes, prostate, pancreas and leukocytes, and is almost undetectable in brain and heart (An et al., 1998a). The third GPCR activated by LPA, PSP24, does not belong to the Edg family (Guo et al., 1996). Expression of PSP24, cloned from Xenopus laevis oocyte, increases the maximal oscillatory chloride currents in oocytes in response to LPA. This response is attenuated in the presence of antisense oligonucleotide targeted against PSP24 -(Guo et al., 1996). Thus, PSP24 may represent an LPA receptor subfamily very distinct from Edg family in terms of amino acid sequence (Guo et al., 1996; Kawasawa et al., 1998).

Via stimulation of these known, and possibly as yet unknown, receptors, LPA produces a plethora of cellular responses through activation of G_{i/o}, G_{q/11}, and G_{12/13} classes of G-proteins (Moolenaar et al., 1997; Goetzl and An, 1998; Nietgen and Durieux, 1998). LPA-mediated effects can be roughly divided into three categories: cell growth-related, cytoskeleton rearrangement-related, and miscellaneous. Responses such as enhanced DNA transcription and cellular proliferation fall into the first category. Increase in DNA synthesis and cell number upon direct application of LPA is observed in a wide variety of cells such as fibroblasts (van Corven et al., 1992; Mao et al., 1998), smooth muscle cells (Cerutis et al., 1997), renal mesangial cells (Gaits et al., 1997b), keratinocytes (Piazza et al., 1995), and astrocytes (Keller et al., 1997), and it is believed that LPA present in serum is one of the factors responsible for serum-induced cell proliferation. Previous studies suggest that LPA-mediated induction of gene transcription involves the activation of MAP kinase-regulated ternary complex factor and rho kinase-regulated serum response factor (Fromm et al., 1997; Moolenaar et al., 1997; Goetzl and An, 1998). These factors, in turn, converge on stimulation of SRE which induces immediate early genes, including c-fos, leading to cell growth (Perkins et al., 1994; Chuprun et al., 1997; Fukushima et al., 1998). It has been demonstrated that LPA-mediated activation of MAP kinase involves the PTX-sensitive stimulation of the G_{i/o} subfamily of G-proteins, as well as of the small molecular weight G-proteins, ras and raf (Howe and Marshall, 1993; van Corven et al., 1993; Hordijk et al., 1994). An additional pathway which may contribute to LPA-induced mitogenesis is the production and secretion of growth factors, such as transforming growth factors alpha and beta, as observed in LPA-stimulated keratinocytes (Piazza et al., 1995). In contrast to the more widely observed proliferative effects, LPA produces inhibition of mitogenesis in some myeloma cell lines (Tigyi et al., 1994). Interestingly, this inhibitory response is coincidental with an increase in cAMP in these cells, and not with the more commonly observed Gᵢ-mediated reduction in cAMP.

The second category of LPA-mediated responses involves effects which are linked to cytoskeletal reorganization, such as actin stress fiber formation, tyrosine phosphorylation of Focal Adhesion Kinase (FAK), chemotaxis, neurite retraction, cell rounding, smooth muscle contraction, and tumor cell invasion (cf. Goetzl and An, 1998). Two observations support the idea that the small molecular weight G-protein, rho, may be the primary mediator of these LPA-mediated effects. First, activation of rho has been linked to stress fiber formation and changes in cell shape (Narumiya et al., 1997). Second, some of the LPA-induced cytoskeletal changes are abolished in the presence of the compounds that inhibit the rho signaling pathway (Ridley and Hall, 1992; Amano et al., 1997; Yoshioka et al., 1998). The G_{12/13} class of G-proteins may couple LPA receptors to rho activation since-some LPA-induced cytoskeietal-responses, such as stress fiber formation and FAK activation, are suppressed in the presence of antibodies directed against G₁₃ (Gohla et al., 1998). Apart from cell growth and cytoskeleton reorganization-related effects, one of the most ubiquitous responses to LPA is enhancement of free intracellular calcium in a wide variety of cells including neurons (Holtsberg et al., 1997). Earlier reports suggested that the G_{q/11} class of G-proteins may be a primary mediator of this response since LPA was shown to induce phosphoinositide hydrolysis, a typical G_{q}-mediated effect, and the calcium mobilization response was insensitive to PTX in fibroblasts (cf. Moolenaar, 1995). However, recent data indicate that some LPA receptors may mobilize calcium by recruiting G_{i/o} G-protiens (Pietruck et al., 1997; An et al., 1998b). It has been suggested that calcium mobilization may play a pivotal role in the stimulation of tumor cell invasion by LPA (Stam et al., 1998). Other cellular responses observed with LPA are platelet aggregation (Schumacher et al., 1979), neurotransmitter secretion (Shiono et al., 1993), potentiation of nicotinic receptor currents (Nishizaki and Sumikawa, 1997), and membrane depolarization via activation of chloride currents (Postma et al., 1996).

Since LPA produces a wide variety of biological effects, it is not surprising that it is proposed to play an important role in various physiological and pathophysiological processes. Probably the foremost in this list is its potential role in wound healing and tissue regeneration. The observation that LPA is released from activated platelets implies that LPA would achieve a high concentration near the site of injury. Furthermore, LPA receptor activation results in responses typically associated with wound healing, such as proliferation of fibroblasts and vascular smooth muscle cells, FAK activation, and stress fiber formation. Together these observations indicate that LPA may be an important mediator of angiogenesis and wound healing. In support of this hypothesis, Piazza et al. (1995) reported that in an in vitro assay, LPA induced proliferation and differentiation of human keratinocytes, and more importantly, topical application of LPA on the skin of hairless mice resulted in epidermal cell proliferation. Similarly, Liliom et al. (1998) demonstrated that LPA was mitogenic in dissociated keratocytes from cornea, and that LPA-like activity in aqueous humor was increased after corneal injury. Thus, an LPA-mimetic may have a beneficial effect in tissue repair and regeneration processes.

LPA could also play a role in pathophysiology of cerebral vasoconstriction. Tigyi et al. (1995) and Yakubu et al. (1997) reported that in newborn piglets, topical application of LPA resulted in vasoconstriction of cerebral arterioles and in prevention of vasodilatation by isoproterenol and hypercapnia. In addition, intrathecal injection of autologous blood led to the production of LPA-like factors in the cerebrospinal fluid. Thus, LPA may be one of the vasoconstrictors generated at the time of intracranial hemorrhage, and may contribute to neurological damage occurring as a consequence of cerebral vasoconstriction. In such a scenario, an antagonist at LPA receptors may be of therapeutic value.

Another-area where LPA could play a modulatory role is cell survival and apoptosis. LPA improves survival of renal-proximal tubular cells (Levine et al., 1997) and macrophages (Koh et al., 1998), and may thus provide protection in ischemia-reperfusion injury. In contrast, it induces apoptosis in hippocampal neurons and may play a role in neurodegenerative diseases such as Alzheimer's disease (Holtsberg et al., 1998). The observation that expression of at least one LPA receptor is developmentally regulated (Hecht et al., 1996), along with LPA's apoptotic ability, suggests a role for LPA in embryonic development where apoptosis is an important process.

There are several pathophysiological processes where LPA could be one of the primary mediators of pathogenesis, such as proliferative diseases, cancer, inflammation, and pulmonary diseases. As mentioned earlier, LPA can produce proliferation of a wide variety of cells and thus may be involved in diseases such as glomerulosclerosis and proliferative vitreoretinopathy (Gaits et al., 1997b; Thoreson et al., 1997)). As for cancer, exposure to LPA induces tumor cell invasiveness in vitro (Stam et al., 1998; Yoshioka et al., 1998)). In addition, it has been observed that LPA production increases in ascites fluids from ovarian cancer patients (Westermann et al., 1998; Xu et al., 1998). These results, coupled with LPA's mitogenic effect, suggest that LPA may be one of the regulators for tumor generation or progression. Several lines of evidence suggest a potential role of LPA as an inflammatory mediator. For example, LPA increases prostaglandin E₂ production in rat mesangial cells (Inoue et al., 1995). Furthermore, LPA induces chemotaxis of fibroblasts and monocytes (Zhou et al., 1995; Sakai et al., 1998) and modulates endothelial permeability (Schulze et al., 1997; Alexander et al., 1998). These cellular changes could lead to inflammation. Finally, LPA may contribute to the development or maintenance of asthma and other obstructive pulmonary disorders since it enhances contractility and proliferation of airway smooth muscles (Cerutis et al., 1997; Toews et al., 1997).

Few in vivo studies have explored the integrated responses to LPA in animals. It has been reported by several groups that LPA induces platelet aggregation in vitro and in vivo (Gerrard et al., 1979; Schumacher et al., 1979; Simon et al., 1984).

Furthermore, intravenous administration of LPA results in species-dependent blood pressure changes (Tokumura et al., 1978; Tokumura et al., 1995). Thus, an LPA-mimetic could be useful in blood coagulation- and blood pressure-related disorders.

In summary, LPA has gained recognition recently as an endogenous mediator of a variety of biological responses. The impressive list of LPA-mediated effects indicates that LPA receptors are attractive targets for therapeutic intervention for several disorders and would be useful to develop drugs with higher specificity and fewer side effects for a wide variety of diseases.

### SUMMARY OF THE INVENTION

This invention provides an isolated nucleic acid encoding a mammalian Edg7 receptor.

This invention further provides a purified mammalian Edg7 receptor protein.

This invention provides a vector comprising a nucleic acid of this invention.

This invention provides a cell comprising a vector of this invention.

This invention provides a membrane preparation isolated from a cell of this invention.

This invention provides a nucleic acid probe comprising at least 15 nucleotides, which probe specifically hybridizes with a nucleic acid encoding a mammalian Edg7 receptor, wherein the probe has a unique sequence corresponding to a sequence present within one of the two strands of the nucleic acid encoding the mammalian Edg7 receptor and contained in plasmid hSNORF3-pCDNA3.1 (ATCC Accession No. 203520).

This invention further provides a nucleic acid probe comprising at least 15 nucleotides, which probe specifically hybridizes with a nucleic acid encoding a mammalian Edg7 receptor, wherein the probe has a unique sequence corresponding to a sequence present within (a) the nucleic acid sequence shown in FIG. 1 (SEQ ID NO: 1) or (b) the reverse complement thereto.

This invention provides an antisense oligonucleotide having a sequence capable of specifically hybridizing to the RNA of this invention, so as to prevent translation of the RNA.

This invention further provides an antisense oligonucleotide having a sequence capable of specifically hybridizing to the genomic DNA of this invention, so as to prevent transcription of the genomic DNA.

This invention provides an antibody capable of binding to a mammalian Edg7 receptor encoded by the nucleic acid of this invention.

This invention provides an agent capable of competitively inhibiting the binding of the antibody of this invention to a mammalian Edg7 receptor.

This invention provides a pharmaceutical composition comprising (a) an amount of the oligonucleotide of this invention capable of passing through a cell membrane and effective to reduce expression of a mammalian Edg7 receptor and (b) a pharmaceutically acceptable carrier capable of passing through the cell membrane.

This invention provides a pharmaceutical composition which comprises an amount of the antibody of this invention effective to block binding of a ligand to a human Edg7 receptor and a pharmaceutically acceptable carrier.

This invention provides a transgenic, nonhuman mammal expressing DNA encoding a mammalian Edg7 receptor of this invention.

This invention further provides a transgenic, nonhuman mammal comprising a homologous recombination knockout of the native mammalian Edg7 receptor.

This invention further provides a transgenic, nonhuman mammal whose genome comprises antisense DNA complementary to the DNA encoding a mammalian Edg7 receptor of this invention so placed within the genome as to be transcribed into antisense mRNA which is complementary to mRNA encoding the mammalian Edg7 receptor and which hybridizes to mRNA encoding the mammalian Edg7 receptor, thereby reducing its translation.

This invention provides a process for identifying a chemical compound which specifically binds to a is mammalian Edg7 receptor which comprises contacting cells containing DNA encoding and expressing on their cell surface the mammalian Edg7 receptor, wherein such cells do not normally express the mammalian Edg7 receptor, with the compound under conditions suitable for binding, and detecting specific binding of the chemical compound to the mammalian Edg7 receptor.

This invention further provides a process for identifying a chemical compound which specifically binds to a mammalian Edg7 receptor which comprises contacting a membrane preparation from cells containing DNA encoding and expressing on their cell surface the mammalian Edg7 receptor, wherein such cells do not normally express the mammalian Edg7 receptor, with the compound under conditions suitable for binding, and detecting specific binding of the chemical compound to the mammalian Edg7 receptor.

This invention further provides a compound identified by one of the above-identified processes.

This invention provides a process involving competitive binding for identifying a chemical compound which specifically binds to a mammalian Edg7 receptor which comprises separately contacting cells expressing on their cell surface the mammalian Edg7 receptor, wherein such cells do not normally express the mammalian Edg7 receptor, with both the chemical compound and a second chemical compound known to bind to the receptor, and with only the second chemical compound, under conditions suitable for binding of both compounds, and detecting specific binding of the chemical compound to the mammalian Edg7 receptor, a decrease in the binding of the second chemical compound to the mammalian Edg7 receptor in the presence of the chemical compound indicating that the chemical compound-binds to the mammalian Edg7 receptor.

This invention further provides a process involving competitive binding for identifying a chemical compound which specifically binds to a mammalian Edg7 receptor which comprises separately contacting a membrane preparation from cells expressing on their cell surface the mammalian Edg7 receptor, wherein such cells do not normally express the mammalian Edg7 receptor, with both the chemical compound and a second chemical compound known to bind to the receptor, and with only the second chemical compound, under conditions suitable for binding of both compounds, and detecting specific binding of the chemical compound to the mammalian Edg7 receptor, a decrease in the binding of the second chemical compound to the mammalian Edg7 receptor in the presence of the chemical compound indicating that the chemical compound binds to the mammalian Edg7 receptor.

This invention further provides a compound identified by one of the above-identified processes.

This invention provides a method of screening a plurality of chemical compounds not known to bind to a mammalian Edg7 receptor to identify a compound which specifically binds to the mammalian Edg7 receptor, which comprises (a)contacting cells transfected with and expressing DNA encoding the mammalian Edg7 receptor with a compound known to bind specifically to the mammalian Edg7 receptor; (b)contacting the preparation of step (a) with the plurality of compounds not known to bind specifically to the mammalian Edg7 receptor, under conditions permitting binding of compounds known to bind to the mammalian Edg7 receptor; (c) determining whether the binding of the compound known to bind to the mammalian Edg7 receptor is reduced in the presence of any compound within the plurality of compounds, relative to the binding of the compound in the absence of the plurality of compounds; and if so (d) separately determining the binding to the mammalian Edg7 receptor of compounds included in the plurality of compounds, so as to thereby identify the compound which specifically binds to the mammalian Edg7 receptor.

This invention further provides a method of screening a plurality of chemical compounds not known to bind to a mammalian Edg7 receptor to identify a compound which specifically binds to the mammalian Edg7 receptor, which comprises (a) contacting a membrane preparation from cells transfected with and expressing DNA encoding the mammalian Edg7 receptor with the plurality of compounds not known to bind specifically to the mammalian Edg7 receptor under conditions permitting binding of compounds known to bind to the mammalian Edg7 receptor; (b) determining whether the binding of a compound known to bind to the mammalian Edg7 receptor is reduced in the presence of any compound within the plurality of compounds, relative to the binding of the compound in the absence of the plurality of compounds; and if so (c) separately determining the binding to the mammalian Edg7 receptor of compounds included in the plurality of compounds, so as to thereby identify the compound which specifically binds to the mammalian Edg7 receptor.

This invention provides a method of detecting expression of a mammalian Edg7 receptor by detecting the presence of mRNA coding for the mammalian Edg7 receptor which comprises obtaining total mRNA from the cell and contacting the mRNA so obtained with the nucleic acid probe of this invention under hybridizing conditions, detecting the presence of mRNA hybridizing to the probe, and thereby detecting the expression of the mammalian Edg7 receptor by the cell.

This invention provides a method of detecting the presence of a mammalian Edg7 receptor on the surface of a cell which comprises contacting the cell with the antibody of this invention under conditions permitting binding of the antibody to the receptor, detecting the presence of the antibody bound to the cell, and thereby detecting the presence of the mammalian Edg7 receptor on the surface of the cell.

This invention provides a method of determining the physiological effects of varying levels of activity of mammalian Edg7 receptors which comprises producing a transgenic, nonhuman mammal of this invention whose levels of mammalian Edg7 receptor activity are varied by use of an inducible promoter which regulates mammalian Edg7 receptor expression.

This invention provides a method of determining the physiological effects of varying levels of activity of mammalian Edg7 receptors which comprises producing a panel of transgenic, nonhuman mammals of this invention each expressing a different amount of mammalian Edg7 receptor.

This invention provides a method for identifying an antagonist capable of alleviating an abnormality wherein the abnormality is alleviated by decreasing the activity of a mammalian Edg7 receptor comprising administering a compound to the transgenic, nonhuman mammal of this invention, and determining whether the compound alleviates the physical and behavioral abnormalities displayed by the transgenic, nonhuman mammal as a result of overactivity of a mammalian Edg7 receptor, the alleviation of the abnormality identifying the compound as an antagonist.

This invention provides an antagonist identified by the above-identified method.

This invention provides a pharmaceutical composition comprising an antagonist of this invention and a pharmaceutically acceptable carrier.

This invention provides a method of treating an abnormality in a subject wherein the abnormality is alleviated by decreasing the activity of a mammalian Edg7 receptor which comprises administering to the subject an effective amount of the pharmaceutical composition of this invention, thereby treating the abnormality.

This invention provides a method for identifying an agonist capable of alleviating an abnormality in a subject wherein the abnormality is alleviated by increasing the activity of a mammalian Edg7 receptor comprising administering a compound to the transgenic, nonhuman mammal of this invention, and determining whether the compound alleviates the physical and behavioral abnormalities displayed by the transgenic, nonhuman mammal, the alleviation of the abnormality identifying the compound as an agonist.

This invention provides an agonist identified by the above-identified method.

This invention provides a pharmaceutical composition comprising an agonist of this invention and a pharmaceutically acceptable carrier.

This invention provides a method of treating an abnormality in a subject wherein the abnormality is alleviated by increasing the activity of a mammalian Edg7 receptor which comprises administering to the subject an effective amount of the pharmaceutical composition of this invention, thereby treating the abnormality.

This invention provides a method for diagnosing a predisposition to a disorder associated with the activity of a specific mammalian allele which comprises: (a) obtaining DNA of subjects suffering from the disorder; (b) performing a restriction digest of the DNA with a panel of restriction enzymes; (c) electrophoretically separating the resulting DNA fragments on a sizing gel; (d) contacting the resulting gel with a nucleic acid probe capable of specifically hybridizing with a unique sequence included within the sequence of a nucleic acid molecule encoding a mammalian Edg7 receptor and labeled with a detectable marker; (e) detecting labeled bands which have hybridized to the DNA encoding a mammalian Edg7 receptor of the invention labeled with a detectable marker to create a unique band pattern specific to the DNA of subjects suffering from the disorder; (f) preparing DNA obtained for diagnosis by steps (a)-(e); and (g) comparing the unique band pattern specific to the DNA of subjects suffering from the disorder from step (e) and the DNA obtained for diagnosis from step (f) to determine whether the patterns are the same or different and to diagnose thereby predisposition to the disorder if the patterns are the same.

This invention provides for a method of preparing the purified mammalian Edg7 receptor of the invention which comprises: (a) culturing cells which express the mammalian Edg7 receptor; (b) recovering the mammalian Edg7 receptor from the cells; and (c) purifying the mammalian Edg7 receptor so recovered.

This invention provides for a method of preparing the purified mammalian Edg7 receptor of the invention which comprises: (a) inserting a nucleic acid encoding the mammalian Edg7 receptor into a suitable vector; (b) introducing the resulting vector into a suitable host cell; (c) placing the resulting cell in suitable condition permitting the production of the mammalian Edg7 receptor; (d) recovering the mammalian Edg7 receptor produced by the resulting cell; and (e) isolating and/or purifying the mammalian Edg7 receptor so recovered.

This invention provides a process for determining whether a chemical compound is a mammalian Edg7 receptor agonist which comprises contacting cells transfected with and expressing DNA encoding the mammalian Edg7 receptor with the compound under conditions permitting the activation of the mammalian Edg7 receptor, and detecting an increase in mammalian Edg7 receptor activity, so as to thereby determine whether the compound is a mammalian Edg7 receptor agonist.

This invention provides a process for determining whether a chemical compound is a mammalian Edg7 receptor antagonist which comprises contacting cells transfected with and expressing DNA encoding the mammalian Edg7 receptor with the compound in the presence of a known mammalian Edg7 receptor agonist, under conditions permitting the activation of the mammalian Edg7 receptor, and detecting a decrease in mammalian Edg7 receptor activity, so as to thereby determine whether the compound is a mammalian Edg7 receptor antagonist.

This invention provides a pharmaceutical composition which comprises an amount of a mammalian Edg7 receptor agonist determined by the process of the invention effective to increase activity of a mammalian Edg7 receptor and a pharmaceutically acceptable carrier.

This invention provides a pharmaceutical composition which comprises an amount of a mammalian Edg7 receptor antagonist determined by the process of the invention effective to reduce activity of a mammalian Edg7 receptor and a pharmaceutically acceptable carrier.

This invention provides a process for determining whether a chemical compound specifically binds to and activates a mammalian Edg7 receptor, which comprises contacting cells producing a second messenger response and expressing on their cell surface the mammalian Edg7 receptor, wherein such cells do not normally express the mammalian Edg7 receptor, with the chemical compound under conditions suitable for activation of the mammalian Edg7 receptor, and measuring the second messenger response in the presence and in the absence of the chemical compound, a change in the second messenger response in the presence of the chemical compound indicating that the compound activates the mammalian Edg7 receptor.

This invention provides a process for determining whether a chemical compound specifically binds to and inhibits activation of a mammalian Edg7 receptor, which comprises separately contacting cells producing a second messenger response and expressing on their cell surface the mammalian Edg7 receptor, wherein such cells do not normally express the mammalian Edg7 receptor, with both the chemical compound and a second chemical compound known to activate the mammalian Edg7 receptor, and with only the second chemical compound, under conditions suitable for activation of the mammalian Edg7 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the mammalian Edg7 receptor.

This invention provides for a compound determined by the process of the invention.

This invention provides for a pharmaceutical composition which comprises an amount of a mammalian Edg7 receptor agonist determined by the process of the invention, effective to increase activity of a mammalian Edg7 receptor and a pharmaceutically acceptable carrier.

This invention provides for a pharmaceutical composition which comprises an amount of a mammalian Edg7 receptor antagonist determined by the process of the invention, effective to reduce activity of a mammalian Edg7 receptor and a pharmaceutically acceptable carrier.

This invention provides for a method of screening a plurality of chemical compounds not known to activate a mammalian Edg7 receptor to identify a compound which activates the mammalian Edg7 receptor which comprises: (a)contacting cells transfected with and expressing the mammalian Edg7 receptor with the plurality of compounds not known to activate the mammalian Edg7 receptor, under conditions permitting activation of the mammalian Edg7 receptor; (b) determining whether the activity of the mammalian Edg7 receptor is increased in the presence of the compounds; and if so (c) separately determining whether the activation of the mammalian Edg7 receptor is increased by each compound included in the plurality of compounds, so as to thereby identify the compound which activates the mammalian Edg7 receptor.

This invention provides for a method of screening a plurality of chemical compounds not known to inhibit the activation of a mammalian Edg7 receptor to identify a compound which inhibits the activation of the mammalian Edg7 receptor, which comprises: (a) contacting cells transfected with and expressing the mammalian Edg7 receptor with the plurality of compounds in the presence of a known mammalian Edg7 receptor agonist, under conditions permitting activation of the mammalian Edg7 receptor; (b) determining whether the activation of the mammalian Edg7 receptor is reduced in the presence of the plurality of compounds, relative to the activation of the mammalian Edg7 receptor in the absence of the plurality of compounds; and if so (c) separately determining the inhibition of activation of the mammalian Edg7 receptor for each compound included in the plurality of compounds, so as to thereby identify the compound which inhibits the activation of the mammalian Edg7 receptor.

This invention provides for a pharmaceutical composition comprising a compound identified by the method of the invention effective to increase mammalian Edg7 receptor activity and a pharmaceutically acceptable carrier.

This invention provides for a pharmaceutical composition comprising a compound identified by the method of the invention effective to decrease mammalian Edg7 receptor activity and a pharmaceutically acceptable carrier.

This invention provides for a method of treating an abnormality in a subject wherein the abnormality is alleviated by increasing the activity of a mammalian Edg7 receptor which comprises administering to the subject an amount of a compound which is a mammalian Edg7 receptor agonist effective to treat the abnormality.

This invention provides for a method of treating an abnormality in a subject wherein the abnormality is alleviated by decreasing the activity of a mammalian Edg7 receptor which comprises administering to the subject an amount of a compound which is a mammalian Edg7 receptor antagonist effective to treat the abnormality.

This invention provides for a process for making a composition of matter which specifically binds to a mammalian Edg7 receptor which comprises identifying a chemical compounds using the process of the invention and then synthesizing the chemical compound or a novel structural and functional analog or homolog thereof.

This invention provides for a process for preparing a pharmaceutical composition which comprises admixing a pharmaceutically acceptable carrier and a pharmaceutically acceptable amount of a chemical compound identified by the process of the invention or a novel structural and functional analog or homolog thereof.

### BRIEF DESCRIPTION OF THE FIGURES

FIGS. 1A-1B

Nucleotide sequence including sequence encoding a human SNORF3 (Edg7) receptor (SEQ ID NO: 1). Putative open reading frames including the shortest open reading frame are indicated by underlining in which two start (ATG) codons (at positions 27-29 and 54-56) and the stop codon (at positions 1086-1088) are underlined. In addition, partial 5′ and 3′ untranslated sequences are shown.

FIGS. 2A-2B

Deduced amino acid sequence (SEQ ID NO: 2) of the human SNORF3 (Edg7) receptor encoded by the longest open reading frame indicated in the nucleotide sequence shown in FIGS. 1A-1B (SEQ ID NO: 1). The seven putative transmembrane (TM) regions are underlined.

FIGS. 3A-3B

Amino acid alignment of human Edg7 (hEdg7) with the other members of the Edg family (SEQ ID NO: 15-SEQ ID NO: 20). Gaps in the alignment are indicated by the “.” symbol. Residues agreeing with the consensus (Cons) are shown in uppercase, while residues differing from the consensus are lowercase.

FIG. 4

Dendrogram of the amino acid alignment of human Edg7 (hEdg7) with the other members of the Edg family, demonstrating that Edg7 and Edg2 cluster closer to each other than to any of the other Edg family members.

FIG. 5

Oleoyl LPA-mediated inositol phosphate (IP) release in Edg7-expressing (A) Cos-7, (B) HEK293, and (C) CHO cells. Edg7 or empty vector (mock) DNA was transfected into the indicated cell lines as described in Materials and Methods. The transfectants were plated into 96-well plates, labeled with [³H]myo-inositol, challenged with varying concentrations of oleoyl LPA and assayed for [³H]IP release as described. A representative experiment is shown.

FIG. 6

Modulation of IP release by several phospholipids in Edg7-expressing Cos-7 cells. The transfectants were plated into 96-well plates, labeled with [³H]myo-inositol, challenged with varying concentrations of oleoyl LPA and assayed for [³H]IP release as described. A representative experiment is shown. PA, phosphatidic acid; LPC, lysophosphatidylcholine; LPE, lysophosphatidylethanolamine; LPG, lysophosphatidylglycerol; S-1-P, sphingosine-1-phosphate; SPC, sphingosylphosphorylcholine.

FIG. 7

A representative example of increase in intracellular calcium by oleoyl LPA (10 μM) and UTP (10 μM) in A) Edg7-expressing, and B) Mock DNA-expressing CHO cells. The cells were loaded with fluo 3, a calcium indicator dye, for 1 h. Subsequently, free intracellular calcium was measured at different time points using Fluorescence Image Plate Reader. The agonists were added at the time indicated by the arrow. Basal fluorescence signal (measured in the absence of agonist) has been subtracted out from the traces.

FIG. 8

Concentration-response relationship for A) oleoyl LPA-induced, and B) UTP-induced increase in intracellular calcium in Edg7- and Mock DNA-expressing CHO cells. The data are presented as mean±SEM (n=6 for oleoyl LPA and n=3 for UTP).

FIG. 9

Increase in intracellular calcium in Edg7-expressing and Mock DNA-expressing CHO cells by A) Stearoyl LPA, B) Palmitoyl LPA, and C) Myristoyl LPA. Data are presented as mean±SEM, n=4 for each agonist.

FIG. 10

Modulation of intracellular calcium in Edg7-expressing and Mock DNA-expressing CHO cells by A) Phosphatidic acid, and B) Sphingosine-1-phosphate. Data are presented as mean±SEM, n=4 for each agonist.

FIG. 11

Modulation of intracellular calcium by various lysophosphatides (n=3) and ATP (n=2). Drugs were added at 10 μM concentration. Data are presented as mean±SEM. LPS, lysophosphatidylserine. For other abbreviations, see figure legend 6.

FIG. 12

Representative traces of oleoyl LPA-induced calcium-activated chloride currents in Xenopus laevis oocytes. The traces labeled Edg7′ were recorded from oocytes injected with mRNA encoding Edg7.

FIG. 13

Averaged calcium-activated chloride current responses to oleoyl LPA (1 μM) in control (uninjected) ooctyes (n=5) and oocytes injected with mRNA encoding Edg7 (n=5).

FIG. 14

Representative traces showing the effect of pertussis toxin (PTX) pretreatment on oleoyl LPA-induced calcium-activated chloride currents in control (uninjected) oocytes and oocytes injected with mRNA encoding Edg7.

FIG. 15

Effect of pertussis toxin (PTX) on oleoyl LPA-induced calcium-activated chloride currents in Edg7-expressing and control oocytes.

FIG. 16

RT-PCR was performed as described on a panel of mRNA extracted from human tissue as indicated at the bottom of the gel. After amplification, PCR reactions were size fractionated on 10% polyacrylamide gels, and stained with SYBR Green I. Images were analyzed using a Molecular Dynamics Storm 860 workstation. The amplified band corresponding to Edg7 is 234 base pairs and is indicated by arrow. RT-PCR indicates a broad distribution of mRNA encoding Edg7. All tissues assayed contained Edg7 mRNA.

FIG. 17

Autoradiograph of solution hybridization/nuclease protection assay to localize Edg7 mRNA extracted from multiple organs. The single band represents mRNA coding for the Edg7 receptor extracted from tissue indicated at the bottom of the gel. mRNA coding for Edg7 is most abundant in: heart, lung, and pancreas. Most tissues, with the exception of adult kidney, liver, pituitary, and substantia nigra contain mRNA encoding Edg7. Integrity of RNA was assessed using hybridization to GAPDH mRNA.

### DETAILED DESCRIPTION OF THE INVENTION

This invention provides a recombinant nucleic acid comprising a nucleic acid encoding a mammalian SNORF3 (Edg7) receptor, wherein the mammalian receptor-encoding nucleic acid hybridizes under high stringency conditions to a nucleic acid encoding a human SNORF3 (Edg7) receptor and having a sequence identical to the sequence of the human SNORF3 (Edg7) receptor-encoding nucleic acid contained in plasmid hSNORF3-pCDNA3.1 (ATCC Accession No. 203520).

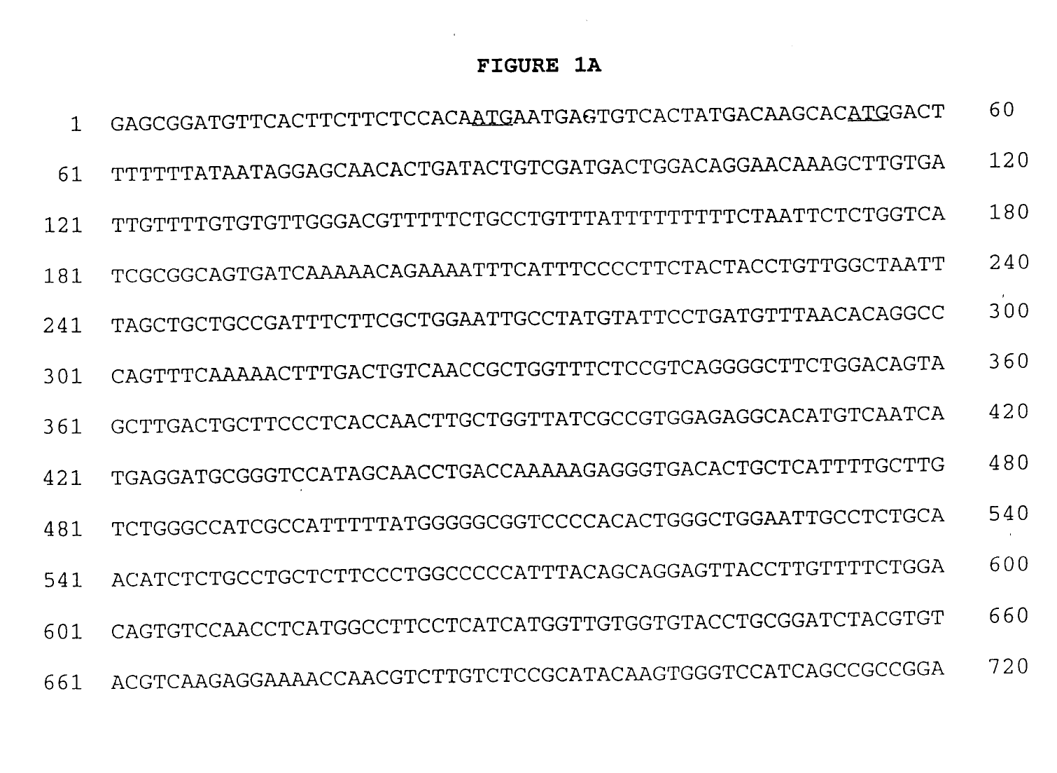

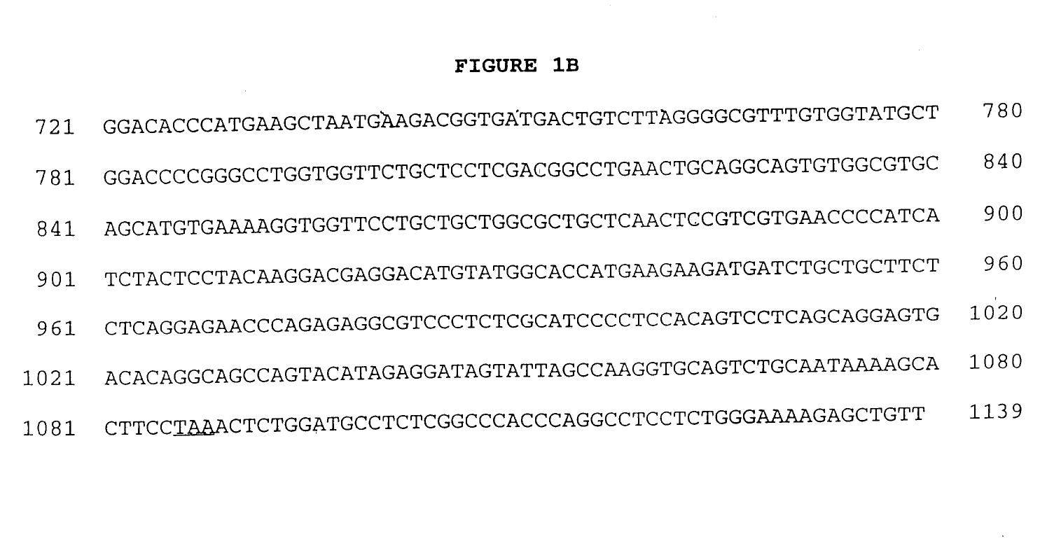

This invention further provides a recombinant nucleic acid comprising a nucleic acid encoding a human SNORF3 receptor, wherein the human SNORF3 receptor comprises an amino acid sequence identical to the sequence of the human SNORF3 receptor encoded by the shortest open reading frame indicated in FIGS. 1A-1B (SEQ ID NO: 1).

The plasmid hSNORF3-pCDNA3.1 was deposited on Dec. 15, 1998, with the American Type Culture Collection (ATCC), 10801 University Blvd., Manassas, Va. 20110-2209, U.S.A. under the provisions of the Budapest Treaty for the International Recognition of the Deposit of Microorganisms for the Purposes of Patent Procedure and was accorded ATCC Accession No. 203520.

Hybridization methods are well known to those of skill in the art. For purposes of this invention, hybridization under high stringency conditions means hybridization performed at 40° C. in a hybridization buffer containing 50% formamide, 5×SSC, 7 mM Tris, 1×Denhardt's, 25 μg/ml salmon sperm DNA; wash at 50° C. in 0.1×SSC, 0.1% SDS.

As used throughout this application, a human Edg7 receptor means a receptor (1) that has the amino acid sequence shown in FIG. 1 (SEQ ID NO: 1) and is activated by LPA; (2) that has an amino acid sequence which is greater than 80% homologous to the sequence shown in FIG. 1 (SEQ ID NO: 1) and is activated by LPA or preferably has an amino acid sequence which is greater than 90% homologous to the sequence shown FIG. 1 (SEQ ID NO: 1) and is activated by LPA; or (3) that is a naturally occurring human receptor whose sequence varies by less than 10 amino acids from the sequence shown in FIG. 1 (SEQ ID NO: 1) and is activated by LPA.

As used throughout this application, a mammalian Edg7 receptor means a receptor which is a human Edg7 receptor or is a species homolog of the human Edg7 receptor found in a species other than man.

Throughout this application, the following standard abbreviations are used to indicate specific nucleotide bases:

A=adenine

G=guanine

C=cytosine

T=thymine

M=adenine or cytosine

R=adenine or guanine

W=adenine or thymine

S=cytosine or guanine

Y=cytosine or thymine

K=guanine or thymine

V=adenine, cytosine, or guanine (not thymine)

H=adenine, cytosine, or thymine (not guanine)

D=adenine, guanine, or thymine (not cytosine)

B=cytosine, guanine, or thymine (not adenine)

N=adenine, cytosine, guanine, or thymine (or other modified base such as inosine)

I=inosine

Furthermore, the term “agonist” is used throughout this application to indicate any peptide or non-peptidyl compound which increases the activity of any of the polypeptides of the subject invention. The term “antagonist” is used throughout this application to indicate any peptide or non-peptidyl compound which decreases the activity of any of the polypeptides of the subject invention.

Furthermore, as used herein, the phrase “pharmaceutically acceptable carrier” means any of the standard pharmaceutically acceptable carriers. Examples include, but are not limited to, phosphate buffered saline, physiological saline, water, and emulsions, such as oil/water emulsions.

It is possible that the mammalian Edg7 receptor genes contain introns and furthermore, the possibility exists that additional introns could exist in coding or non-coding regions. In addition, spliced form(s) of mRNA may encode additional amino acids either upstream of the currently defined starting methionine or within the coding region. Further, the existence and use of alternative exons is possible, whereby the mRNA may encode different amino acids within the region comprising the exon. In addition, single amino acid substitutions may arise via the mechanism of RNA editing such that the amino acid sequence of the expressed protein is different than that encoded by the original gene. (Burns et al., 1996; Chu et al., 1996). Such variants may exhibit pharmacologic properties differing from the polypeptide encoded by the original gene.

This invention provides splice variants of the mammalian Edg7 receptors disclosed herein. This invention further provides for alternate translation initiation sites and alternately spliced or edited variants of nucleic acids encoding the mammalian Edg7 receptors of this invention.

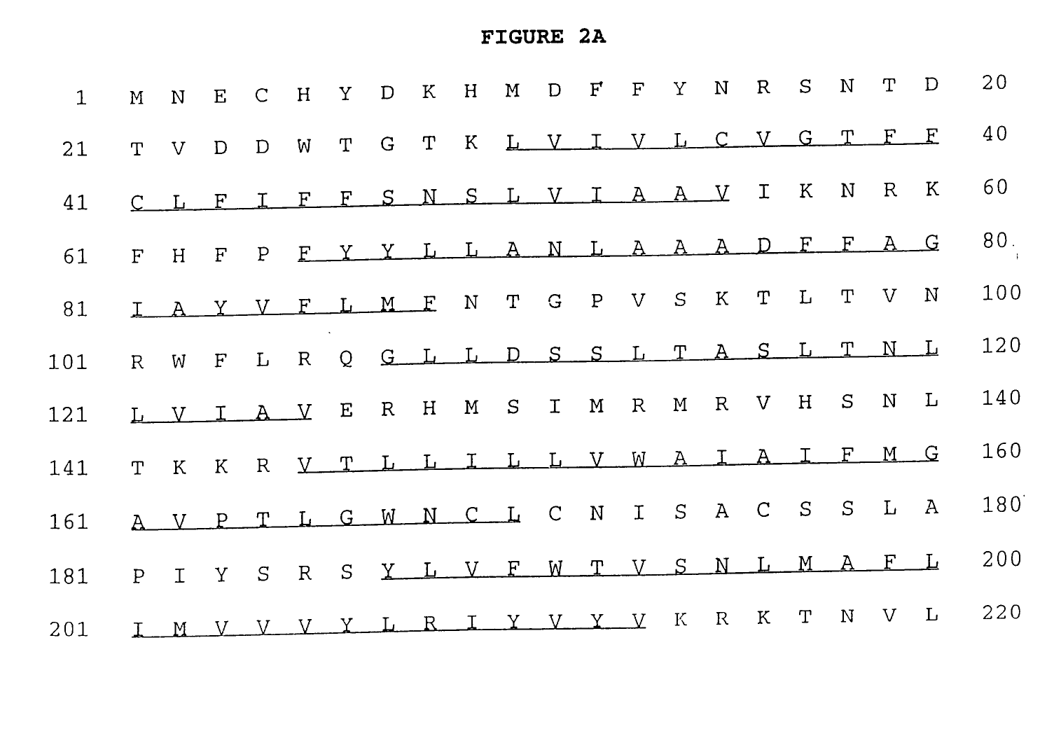

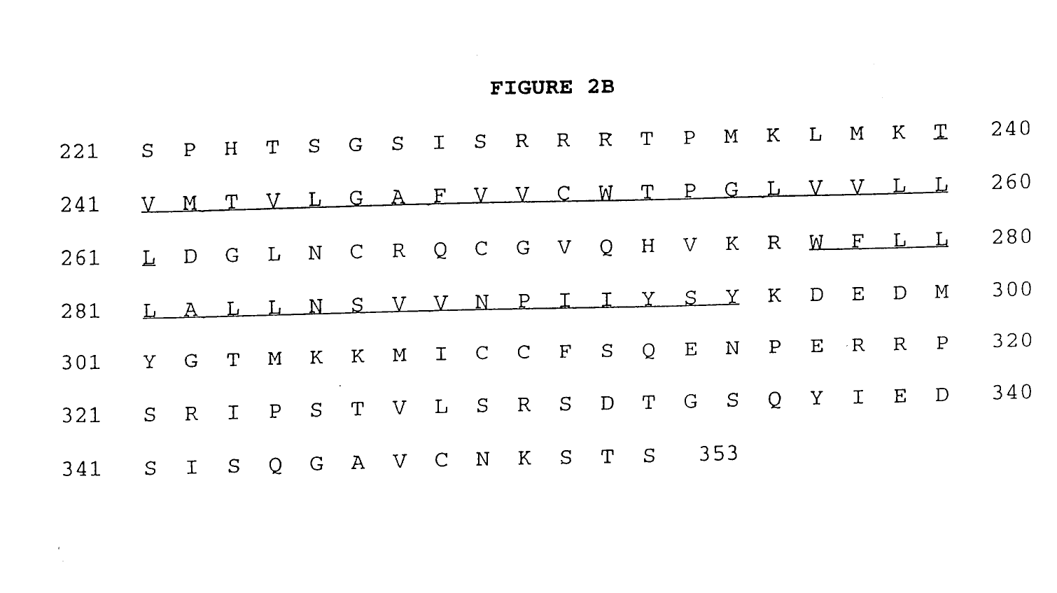

The nucleic acids of the subject invention also include nucleic acid analogs of the human Edg7 receptor genes, wherein the human Edg7 receptor gene comprises the nucleic acid sequence shown in FIG. 1 or contained in plasmid hSNORF3-pCDNA3.1 (ATCC Accession No. 203520). Nucleic acid analogs of the human Edg7 receptor genes differ from the human Edg7 receptor genes described herein in terms of the identity or location of one or more nucleic acid bases (deletion analogs containing less than all of the nucleic acid bases shown in FIG. 1 or contained in plasmid hSNORF3-pCDNA3.1, substitution analogs wherein one or more nucleic acid bases shown in FIG. 1 or contained in plasmid hSNORF3-pCDNA3.1, are replaced by other nucleic acid bases, and addition analogs, wherein one or more nucleic acid bases are added to a terminal or medial portion of the nucleic acid sequence) and which encode proteins which share some or all of the properties of the proteins encoded by the nucleic acid sequences shown in FIG. 1 or contained in plasmid hSNORF3-pCDNA3.1. In one embodiment of the present invention, the nucleic acid analog encodes a protein which has an amino acid sequence identical to that shown in FIG. 2 or encoded by the nucleic acid sequence contained in plasmid hSNORF3-pCDNA3.1. In another embodiment, the nucleic acid analog encodes a protein having an amino acid sequence which differs from the amino acid sequences shown in FIG. 2 or encoded by the nucleic acid contained in plasmid hSNORF3-pCDNA3.1. In a further embodiment, the protein encoded by the nucleic acid analog has a function which is the same as the function of the receptor proteins having the amino acid sequence shown in FIG. 2. In another embodiment, the function of the protein encoded by the nucleic acid analog differs from the function of the receptor protein having the amino acid sequence shown in FIG. 2. In another embodiment, the variation in the nucleic acid sequence occurs within the transmembrane (TM) region of the protein. In a further embodiment, the variation in the nucleic acid sequence occurs outside of the TM region.

This invention further provides nucleic acid which is degenerate with respect to the DNA encoding any of the polypeptides described herein. In an embodiment, the nucleic acid comprises a nucleotide sequence which is degenerate with respect to the nucleotide sequence shown in FIG. 1 (SEQ ID NO: 1) or the nucleotide sequence contained in the plasmid hSNORF3-pCDNA3.1, that is, a nucleotide sequence which is translated into the same amino acid sequence.

This invention also encompasses DNAs and cDNAs which encode amino acid sequences which differ from those of the polypeptides of this invention, but which should not produce phenotypic changes. Alternately, this invention also encompasses DNAs, cDNAs, and RNAs which hybridize to the DNA, cDNA, and RNA of the subject invention. Hybridization methods are well known to those of skill in the art.

The nucleic acids of the subject invention also include nucleic acid molecules coding for polypeptide analogs, fragments or derivatives of antigenic polypeptides which differ from naturally-occurring forms in terms of the identity or location of one or more amino acid residues (deletion analogs containing less than all of the residues specified for the protein, substitution analogs wherein one or more residues specified are replaced by other residues and addition analogs wherein one or more amino acid residues is added to a terminal or medial portion of the polypeptides) and which share some or all properties of naturally-occurring forms. These molecules include: the incorporation of codons “preferred” for expression by selected non-mammalian hosts; the provision of sites for cleavage by restriction endonuclease enzymes; and the provision of additional initial, terminal or intermediate DNA sequences that facilitate construction of readily expressed vectors. The creation of polypeptide analogs is well known to those of skill in the art (Spurney, R. F. et al. (1997); Fong, T. M. et al. (1995); Underwood, D. J. et al. (1994); Graziano, M. P. et al. (1996); Guan X. M. et al. (1995)).

The modified polypeptides of this invention may be transfected into cells either transiently or stably using methods well-known in the art, examples of which are disclosed herein. This invention also provides for binding assays using the modified polypeptides, in which the polypeptide is expressed either transiently or in stable cell lines. This invention further provides a compound identified using a modified polypeptide in a binding assay such as the binding assays described herein.

The nucleic acids described and claimed herein are useful for the information which they provide concerning the amino acid sequence of the polypeptide and as products for the large scale synthesis of the polypeptides by a variety of recombinant techniques. The nucleic acid molecule is useful for generating new cloning and expression vectors, transformed and transfected prokaryotic and eukaryotic host cells, and new and useful methods for cultured growth of such host cells capable of expression of the polypeptide and related products.

This invention also provides an isolated nucleic acid encoding species homologs of the Edg7 receptors encoded by the nucleic acid sequence shown in FIG. 1 (SEQ ID NO: 1) or encoded by the plasmid hSNORF3-pCDNA3.1. In one embodiment, the nucleic acid encodes a mammalian Edg7 receptor homolog which has substantially the same amino acid sequence as does the Edg7 receptor encoded by the plasmid hSNORF3-pCDNA3.1. In another embodiment, the nucleic acid encodes a mammalian Edg7 receptor homolog which has above 75% amino acid identity to the Edg7 receptor encoded by the plasmid hSNORF3-pCDNA3.1; preferably above 85% amino acid identity to the Edg7 receptor encoded by the plasmid hSNORF3-pCDNA3.1; most preferably above 95% amino acid identity to the Edg7 receptor encoded by the plasmid hSNORF3-pCDNA3.1. In another embodiment, the mammalian Edg7 receptor homolog has above 70% nucleic acid identity to the Edg7 receptor gene contained in plasmid hSNORF3-pCDNA3.1; preferably above 80% nucleic acid identity to the Edg7 receptor gene contained in the plasmid hSNORF3-pCDNA3.1; more preferably above 90% nucleic acid identity to the Edg7 receptor gene contained in the plasmid hSNORF3-pCDNA3.1. Examples of methods for isolating and purifying species homologs are described elsewhere (e.g., U.S. Pat. No. 5,602,024, WO 94/14957, WO 97/26853, WO 98/15570).

This invention provides an isolated nucleic acid encoding a modified mammalian Edg7 receptor, which differs from a mammalian Edg7 receptor by having an amino acid(s) deletion, replacement, or addition in the third intracellular domain.

This invention provides an isolated nucleic acid encoding a mammalian Edg7 receptor. In one embodiment, the nucleic acid is DNA. In another embodiment, the DNA is cDNA. In another embodiment, the DNA is genomic DNA. In another embodiment, the nucleic acid is RNA. In another embodiment, the mammalian Edg7 receptor is a human Edg7 receptor. In another embodiment, the human Edg7 receptor has an amino acid sequence identical to that encoded by the plasmid hSNORF3-pCDNA3.1 (ATCC Accession No. 203520). In another embodiment, the human Edg7 receptor has an amino acid sequence identical to the amino acid sequence shown in FIG. 2 (SEQ ID NO: 2).

This invention provides a purified mammalian Edg7 receptor protein. In one embodiment, the Edg7 receptor protein is a human Edg7 receptor protein.

This invention provides a vector comprising the nucleic acid of this invention. This invention further provides a vector adapted for expression in a cell which comprises the regulatory elements necessary for expression of the nucleic acid in the cell operatively linked to the nucleic acid encoding the receptor so as to permit expression thereof, wherein the cell is a bacterial, amphibian, yeast, insect or mammalian cell. In one embodiment, the vector is a baculovirus. In another embodiment, the vector is a plasmid.

This invention provides a plasmid designated hSNORF3-pCDNA3.1 (ATCC Accession No. 203520).

This invention further provides for any vector or plasmid which comprises modified untranslated sequences, which are beneficial for expression in desired host cells or for use in binding or functional assays. For example, a vector or plasmid with untranslated sequences of varying lengths may express differing amounts of the polypeptide depending upon the host cell used. In an embodiment, the vector or plasmid comprises the coding sequence of the polypeptide and the regulatory elements necessary for expression in the host cell.

This invention provides for a cell comprising the vector of this invention. In one embodiment, the cell is a non-mammalian cell. In one embodiment, the non-mammalian cell is a Xenopus oocyte cell or a Xenopus melanophore cell. In another embodiment, the cell is a mammalian cell. In another embodiment, the cell is a COS-7 cell, a 293 human embryonic kidney cell, a NIH-3T3 cell, a LM(tk-) cell, a mouse Y1cell, or a CHO cell. In another embodiment, the cell is an insect cell. In another embodiment, the insect cell is an Sf9 cell, an Sf21 cell or a Trichoplusia ni 5B-4 cell.

This invention provides a membrane preparation isolated from the cell of this invention.

This invention provides for a nucleic acid probe comprising at least 15 nucleotides, which probe specifically hybridizes with a nucleic acid encoding a mammalian Edg7 receptor, wherein the probe has a unique sequence corresponding to a sequence present within one of the two strands of the nucleic acid encoding the mammalian Edg7 receptor and contained in plasmid hSNORF3-pCDNA3.1 (ATCC Accession No. 203520).

This invention provides for a nucleic acid probe comprising at least 15 nucleotides, which probe specifically hybridizes with a nucleic acid encoding a mammalian Edg7 receptor, wherein the probe has a unique sequence corresponding to a sequence present within (a) the nucleic acid sequence shown in FIG. 1 (SEQ ID NO: 1) or (b) the reverse complement thereto. In one embodiment, the nucleic acid is DNA. In another embodiment, the nucleic acid is RNA.

As used herein, the phrase “specifically hybridizing” means the ability of a nucleic acid molecule to recognize a nucleic acid sequence complementary to its own and to form double-helical segments through hydrogen bonding between complementary base pairs.

The nucleic acids-of this invention may be used as probes to obtain homologous nucleic acids from other species and to detect the existence of nucleic acids having complementary sequences in samples.

The nucleic acids may also be used to express the receptors they encode in transfected cells.

The use of a constitutively active receptor encoded by SNORF3 (Edg7) either occurring naturally without further modification or after appropriate point mutations, deletions or the like, allows screening for antagonists and in vivo use of such antagonists to attribute a role to receptor SNORF3 (Edg7) without prior knowledge of the endogenous ligand.

Use of the nucleic acids further enables elucidation of possible receptor diversity and of the existence of multiple subtypes within a family of receptors of which SNORF3 (Edg7) is a member.

Methods of transfecting cells e.g. mammalian cells, with such nucleic acid to obtain cells in which the receptor is expressed on the surface of the cell are well known in the art. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.)

Such transfected cells may also be used to test compounds and screen compound libraries to obtain compounds which bind to the SNORF3 (Edg7) receptor, as well as compounds which activate or inhibit activation of functional responses in such cells, and therefore are likely to do so in vivo. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.)

This invention further provides an antibody capable of binding to a mammalian receptor encoded by a nucleic acid encoding a mammalian receptor. In one embodiment, the mammalian receptor is a human receptor. This invention also provides an agent capable of competitively inhibiting the binding of the antibody to a mammalian receptor. In one embodiment, the antibody is a monoclonal antibody or antisera.

This invention also provides a nucleic acid probe comprising at least 15 nucleotides, which probe specifically hybridizes with a nucleic acid encoding a mammalian receptor, wherein the probe has a sequence corresponding to a unique sequence present within one of the two strands of the nucleic acid encoding the mammalian receptor and is contained in plasmid hSNORF3-pCDNA3.1 (ATCC Accession No. 203520). This invention also provides a nucleic acid probe comprising at least 15 nucleotides, which probe specifically hybridizes with a nucleic acid encoding a mammalian receptor, wherein the probe has a sequence corresponding to a unique sequence present within (a) the nucleic acid sequence shown in FIGS. 1A-1B (SEQ ID NO: 1) or (b) the reverse complement thereto. In one embodiment, the nucleic acid is DNA. In another embodiment, the nucleic acid is RNA.

Methods of preparing and employing antisense oligonucleotides, antibodies, nucleic acid probes and transgenic animals directed to the SNORF3 (Edg7) receptor are well known in the art. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.)

This invention provides for an antisense oligonucleotide having a sequence capable of specifically hybridizing to the RNA of this invention, so as to prevent translation of the RNA. This invention further provides for an antisense oligonucleotide having a sequence capable of specifically hybridizing to the genomic DNA of this invention, so as to prevent transcription of the genomic DNA. In one embodiment, the oligonucleotide comprises chemically modified nucleotides or nucleotide analogues.

This invention provides for an antibody capable of binding to a mammalian Edg7 receptor encoded by the nucleic acid of this invention. In one embodiment, the mammalian Edg7 receptor is a human Edg7 receptor.

This invention provides an agent capable of competitively inhibiting the binding of the antibody of this invention to a mammalian Edg7 receptor. In one embodiment, the antibody is a monoclonal antibody or antisera.

This invention provides a pharmaceutical composition comprising (a) an amount of the oligonucleotide of this invention capable of passing through a cell membrane and effective to reduce expression of a mammalian Edg7 receptor and (b) a pharmaceutically acceptable carrier capable of passing through the cell membrane.

In one embodiment, the oligonucleotide is coupled to a substance which inactivates mRNA. In another embodiment, the substance which inactivates mRNA is a ribozyme. In another embodiment, the pharmaceutically acceptable carrier comprises a structure which binds to a mammalian Edg7 receptor on a cell capable of being taken up by the cells after binding to the structure. In another embodiment, the pharmaceutically acceptable carrier is capable of binding to a mammalian Edg7 receptor which is specific for a selected cell type.

This invention provides a pharmaceutical composition which comprises an amount of the antibody of this invention effective to block binding of a ligand to a human Edg7 receptor and a pharmaceutically acceptable carrier.

This invention provides a transgenic, nonhuman mammal expressing DNA encoding a mammalian Edg7 receptor of this invention. This invention provides a transgenic, nonhuman mammal comprising a homologous recombination knockout of the native mammalian Edg7 receptor. This invention further provides a transgenic, nonhuman mammal whose genome comprises antisense DNA complementary to the DNA encoding a mammalian Edg7 receptor of this invention so placed within the genome as to be transcribed into antisense mRNA which is complementary to mRNA encoding the mammalian Edg7 receptor and which hybridizes to mRNA encoding the mammalian Edg7 receptor, thereby reducing its translation. In one embodiment, the DNA encoding the mammalian Edg7 receptor additionally comprises an inducible promoter. In another embodiment, the DNA encoding the mammalian Edg7 receptor additionally comprises tissue specific regulatory elements. In another embodiment, the transgenic, nonhuman mammal is a mouse.

This invention provides for a process for identifying a chemical compound which specifically binds to a mammalian Edg7 receptor which comprises contacting cells containing DNA encoding and expressing on their cell surface the mammalian Edg7 receptor, wherein such cells do not normally express the mammalian Edg7 receptor, with the compound under conditions suitable for binding, and detecting specific binding of the chemical compound to the mammalian Edg7 receptor. This invention further provides for a process for identifying a chemical compound which specifically binds to a mammalian Edg7 receptor which comprises contacting a membrane preparation from cells containing DNA encoding and expressing on their cell surface the mammalian Edg7 receptor, wherein such cells do not normally express the mammalian Edg7 receptor, with the compound under conditions suitable for binding, and detecting specific binding of the chemical compound to the mammalian Edg7 receptor.

In one embodiment, the mammalian Edg7 receptor is a human Edg7 receptor. In another embodiment, the mammalian Edg7 receptor has substantially the same amino acid sequence as the human Edg7 receptor encoded by plasmid hSNORF3-pCDNA3.1 (ATCC Accession No. 203520). In another embodiment, the mammalian Edg7 receptor has substantially the same amino acid sequence as that shown in FIG. 2 (SEQ ID NO: 2). In another embodiment, the mammalian Edg7 receptor has the amino acid sequence shown in FIG. 2 (SEQ ID NO: 2). In one embodiment, the compound is not previously known to bind to a mammalian Edg7 receptor. In one embodiment, the cell is an insect cell. In one embodiment, the cell is a mammalian cell. In another embodiment, the cell is nonneuronal in origin. In another embodiment, the nonneuronal cell is a COS-7 cell, 293 human embryonic kidney cell, a CHO cell, a NIH-3T3 cell, a mouse Y1 cell, or a LM (tk-) cell. In another embodiment, the compound is a compound not previously known to bind to a mammalian Edg7 receptor. This invention provides a compound identified by the above-identified process of this invention.

This invention provides a process involving competitive binding for identifying a chemical compound which specifically binds to a mammalian Edg7 receptor which comprises separately contacting cells expressing on their cell surface the mammalian Edg7 receptor, wherein such cells do not normally express the mammalian Edg7 receptor, with both the chemical compound and a second chemical compound known to bind to the receptor, and with only the second chemical compound, under conditions suitable for binding of both compounds, and detecting specific binding of the chemical compound to the mammalian Edg7 receptor, a decrease in the binding of the second chemical compound to the mammalian Edg7 receptor in the presence of the chemical compound indicating that the chemical compound binds to the mammalian Edg7 receptor.

This invention provides a process involving competitive binding for identifying a chemical compound which specifically binds to a mammalian Edg7 receptor which comprises separately contacting a membrane preparation from cells expressing on their cell surface the mammalian Edg7 receptor, wherein such cells do not normally express the mammalian Edg7 receptor, with both the chemical compound and a second chemical compound known to bind to the receptor, and with only the second chemical compound, under conditions suitable for binding of both compounds, and detecting specific binding of the chemical compound to the mammalian Edg7 receptor, a decrease in the binding of the second chemical compound to the mammalian Edg7 receptor in the presence of the chemical compound indicating that the chemical compound binds to the mammalian Edg7 receptor.

In one embodiment, the mammalian Edg7 receptor is a human Edg7 receptor. In another embodiment, the cell is an insect cell. In another embodiment, the cell is a mammalian cell. In another embodiment, the cell is nonneuronal in origin. In another embodiment, the nonneuronal cell is a COS-7 cell, 293 human embryonic kidney cell, a CHO cell, a NIH-3T3 cell, a mouse Y1 cell, or a LM(tk-) cell. In another embodiment, the compound is not previously known to bind to a mammalian Edg7 receptor. This invention provides for a compound identified by the above-identified process of this invention.

This invention provides for a method of screening a plurality of chemical compounds not known to bind to a mammalian Edg7 receptor to identify a compound which specifically binds to the mammalian Edg7 receptor, which comprises (a) contacting cells transfected with and expressing DNA encoding the mammalian Edg7 receptor with a compound known to bind specifically to the mammalian Edg7 receptor; (b) contacting the preparation of step (a) with the plurality of compounds not known to bind specifically to the mammalian Edg7 receptor, under conditions permitting binding of compounds known to bind to the mammalian Edg7 receptor; (c) determining whether the binding of the compound known to bind to the mammalian Edg7 receptor is reduced in the presence of any compound within the plurality of compounds, relative to the binding of the compound in the absence of the plurality of compounds; and if so (d) separately determining the binding to the mammalian Edg7 receptor of compounds included in the plurality of compounds, so as to thereby identify the compound which specifically binds to the mammalian Edg7 receptor.

This invention provides a method of screening a plurality of chemical compounds not known to bind to a mammalian Edg7 receptor to identify a compound which specifically binds to the mammalian Edg7 receptor, which comprises (a) contacting a membrane preparation from cells transfected with and expressing DNA encoding the mammalian Edg7 receptor with the plurality of compounds not known to bind specifically to the mammalian Edg7 receptor under conditions permitting binding of compounds known to bind to the mammalian Edg7 receptor; (b) determining whether the binding of a compound known to bind to the mammalian Edg7 receptor is reduced in the presence of any compound within the plurality of compounds, relative to the binding of the compound in the absence of the plurality of compounds; and if so (c) separately determining the binding to the mammalian Edg7 receptor of compounds included in the plurality of compounds, so as to thereby identify the compound which specifically binds to the mammalian Edg7 receptor.

In one embodiment, the mammalian Edg7 receptor is a human Edg7 receptor. In another embodiment, the cell is a mammalian cell. In another embodiment, the mammalian cell is non-neuronal in origin. In a further embodiment, the non-neuronal cell is a COS-7 cell, a 293 human embryonic kidney cell, a LM(tk-) cell, a CHO cell, a mouse Y1 cell, or an NIH-3T3 cell.

This invention provides a method of detecting expression of a mammalian Edg7 receptor by detecting the presence of mRNA coding for the mammalian Edg7 receptor which comprises obtaining total mRNA from the cell and contacting the mRNA so obtained with the nucleic acid probe of this invention under hybridizing conditions, detecting the presence of mRNA hybridizing to the probe, and thereby detecting the expression of the mammalian Edg7 receptor by the cell.

This invention provides for a method of detecting the presence of a mammalian Edg7 receptor on the surface of a cell which comprises contacting the cell with the antibody of this invention under conditions permitting binding of the antibody to the receptor, detecting the presence of the antibody bound to the cell, and thereby detecting the presence of the mammalian Edg7 receptor on the surface of the cell.

This invention provides a method of determining the physiological effects of varying levels of activity of mammalian Edg7 receptors which comprises producing a transgenic, nonhuman mammal of this invention whose levels of mammalian Edg7 receptor activity are varied by use of an inducible promoter which regulates mammalian Edg7 receptor expression.

This invention provides a method of determining the physiological effects of varying levels of activity of mammalian Edg7 receptors which comprises producing a panel of transgenic, nonhuman mammals of this invention each expressing a different amount of mammalian Edg7 receptor.

This invention provides method for identifying an antagonist capable of alleviating an abnormality wherein the abnormality is alleviated by decreasing the activity of a mammalian Edg7 receptor comprising administering a compound to the transgenic, nonhuman mammal of this invention, and determining whether the compound alleviates the physical and behavioral abnormalities displayed by the transgenic, nonhuman mammal as a result of overactivity of a mammalian Edg7 receptor, the alleviation of the abnormality identifying the compound as an antagonist. In one embodiment, the mammalian Edg7 receptor is a human Edg7 receptor. The invention provides an antagonist identified by the above-identified method of this invention. This invention provides a pharmaceutical composition comprising an antagonist of this invention and a pharmaceutically acceptable carrier. This invention provides a method of treating an abnormality in a subject wherein the abnormality is alleviated by decreasing the activity of a mammalian Edg7 receptor which comprises administering to the subject an effective amount of the pharmaceutical composition of this invention, thereby treating the abnormality.

This invention provides a method for identifying an agonist capable of alleviating an abnormality in a subject wherein the abnormality is alleviated by increasing the activity of a mammalian Edg7 receptor comprising administering a compound to the transgenic, nonhuman mammal of this invention, and determining whether the compound alleviates the physical and behavioral abnormalities displayed by the transgenic, nonhuman mammal, the alleviation of the abnormality identifying the compound as an agonist. In one embodiment, the mammalian Edg7 receptor is a human Edg7 receptor. This invention provides an agonist identified by the above-identified method of this invention. This invention provides a pharmaceutical composition comprising an agonist identified by the method of this invention and a pharmaceutically acceptable carrier.

This invention provides a method of treating an abnormality in a subject wherein the abnormality is alleviated by increasing the activity of a mammalian Edg7 receptor which comprises administering to the subject an effective amount of the pharmaceutical composition of this invention, thereby treating the abnormality.

This invention provides a method for diagnosing a predisposition to a disorder associated with the activity of a specific mammalian allele which comprises: (a) obtaining DNA of subjects suffering from the disorder; (b) performing a restriction digest of the DNA with a panel of restriction enzymes; (c) electrophoretically separating the resulting DNA fragments on a sizing gel; (d) contacting the resulting gel with a nucleic acid probe capable of specifically hybridizing with a unique sequence included within the sequence of a nucleic acid molecule encoding a mammalian Edg7 receptor and labeled with a detectable marker; (e) detecting labeled bands which have hybridized to the DNA encoding a mammalian Edg7 receptor of this invention labeled with a detectable marker to create a unique band pattern specific to the DNA of subjects suffering from the disorder; (f) preparing DNA obtained for diagnosis by steps (a)-(e); and (g) comparing the unique band pattern specific to the DNA of subjects suffering from the disorder from step (e) and the DNA obtained for diagnosis from step (f) to determine whether the patterns are the same or different and to diagnose thereby predisposition to the disorder if the patterns are the same.

In one embodiment, the disorder is a disorder associated with the activity of a specific mammalian allele is diagnosed.

This invention provides a method of preparing the purified mammalian Edg7 receptor of this invention which comprises: (a) culturing cells which express the mammalian Edg7 receptor; (b) recovering the mammalian Edg7 receptor from the cells; and (c) purifying the mammalian Edg7 receptor so recovered.

This invention provides a method of preparing the purified mammalian Edg7 receptor of this invention which comprises: (a) inserting a nucleic acid encoding the mammalian Edg7 receptor into a suitable vector; (b) introducing the resulting vector into a suitable host cell; (c) placing the resulting cell in suitable condition permitting the production of the mammalian Edg7 receptor; (d) recovering the mammalian Edg7 receptor produced by the resulting cell; and (e) isolating and/or purifying the mammalian Edg7 receptor so recovered.

This invention provides a process for determining whether a chemical compound is a mammalian Edg7 receptor agonist which comprises contacting cells transfected with and expressing DNA encoding the mammalian Edg7 receptor with the compound under conditions permitting the activation of the mammalian Edg7 receptor, and detecting an increase in mammalian Edg7 receptor activity, so as to thereby determine whether the compound is a mammalian Edg7 receptor agonist.

This invention provides a process for determining whether a chemical compound is a mammalian Edg7 receptor antagonist which comprises contacting cells transfected with and expressing DNA encoding the mammalian Edg7 receptor with the compound in the presence of a known mammalian Edg7 receptor agonist, under conditions permitting the activation of the mammalian Edg7 receptor, and detecting a decrease in mammalian Edg7 receptor activity, so as to thereby determine whether the compound is a mammalian Edg7 receptor antagonist.

In one embodiment, the mammalian Edg7 receptor is a human Edg7 receptor.

This invention provides a pharmaceutical composition which comprises an amount of a mammalian Edg7 receptor agonist determined by the process of this invention effective to increase activity of a mammalian Edg7 receptor and a pharmaceutically acceptable carrier. In one embodiment, the mammalian Edg7 receptor agonist is not previously known.

This invention provides a pharmaceutical composition which comprises an amount of a mammalian Edg7 receptor antagonist determined by the process of this invention effective to reduce activity of a mammalian Edg7 receptor and a pharmaceutically acceptable carrier. In one embodiment, the mammalian Edg7 receptor antagonist is not previously known.

This invention provides a process for determining whether a chemical compound specifically binds to and activates a mammalian Edg7 receptor, which comprises contacting cells producing a second messenger response and expressing on their cell surface the mammalian Edg7 receptor, wherein such cells do not normally express the mammalian Edg7 receptor, with the chemical compound under conditions suitable for activation of the mammalian Edg7 receptor, and measuring the second messenger response in the presence and in the absence of the chemical compound, a change in the second messenger response in the presence of the chemical compound indicating that the compound activates the mammalian Edg7 receptor.

In one embodiment, the second messenger response comprises chloride channel activation and the change in second messenger is an increase in the level of chloride current. In another embodiment, the second messenger response comprises change in intracellular calcium levels and the change in second messenger is an increase in the measure of intracellular calcium. In another embodiment, the second messenger response comprises release of inositol phosphate and the change in second messenger is an increase in the level of inositol phosphate.

This invention provides a process for determining whether a chemical compound specifically binds to and inhibits activation of a mammalian Edg7 receptor, which comprises separately contacting cells producing a second messenger response and expressing on their cell surface the mammalian Edg7 receptor, wherein such cells do not normally express the mammalian Edg7 receptor, with both the chemical compound and a second chemical compound known to activate the mammalian Edg7 receptor, and with only the second chemical compound, under conditions suitable for activation of the mammalian Edg7 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the mammalian Edg7 receptor.

In one embodiment, the second messenger response comprises chloride channel activation and the change in second messenger response is a smaller increase in the level of chloride current in the presence of both the chemical compound and the second chemical compound than in the presence of only the second chemical compound. In another embodiment, the second messenger response comprises change in intracellular calcium levels and the change in second messenger response is a smaller increase in the measure of intracellular calcium in the presence of both the chemical compound and the second chemical compound than in the presence of only the second chemical compound. In another embodiment, the second messenger response comprises release of inositol phosphate and the change in second messenger response is a smaller increase in the level of inositol phosphate in the presence of both the chemical compound and the second chemical compound than in the presence of only the second chemical compound.

In one embodiment, the mammalian Edg7 receptor is a human Edg7 receptor. In another embodiment, the cell is an insect cell. In another embodiment, the cell is a mammalian cell. In another embodiment, the mammalian cell is nonneuronal in origin. In another embodiment, the nonneuronal cell is a COS-7 cell, CHO cell, 293 human embryonic kidney cell, NIH-3T3 cell or LM(tk-) cell. In another embodiment, the compound is not previously known to bind to a mammalian Edg7 receptor.

This invention provides a compound determined by the process of this invention.

This invention provides a pharmaceutical composition which comprises an amount of a mammalian Edg7 receptor agonist determined by the process of this invention effective to increase activity of a mammalian Edg7 receptor and a pharmaceutically acceptable carrier. In one embodiment, the mammalian Edg7 receptor agonist is not previously known.

This invention provides a pharmaceutical composition which comprises an amount of a mammalian Edg7 receptor antagonist determined by the process of this invention, effective to reduce activity of a mammalian Edg7 receptor and a pharmaceutically acceptable carrier. In one embodiment, the mammalian Edg7 receptor antagonist is not previously known.

This invention provides a method of screening a plurality of chemical compounds not known to activate a mammalian Edg7 receptor to identify a compound which activates the mammalian Edg7 receptor which comprises: (a) contacting cells transfected with and expressing the mammalian Edg7 receptor with the plurality of compounds not known to activate the mammalian Edg7 receptor, under conditions permitting activation of the mammalian Edg7 receptor; (b) determining whether the activity of the mammalian Edg7 receptor is increased in the presence of the compounds; and if so (c) separately determining whether the activation of the mammalian Edg7 receptor is increased by each compound included in the plurality of compounds, so as to thereby identify the compound which activates the mammalian Edg7 receptor. In one embodiment, the mammalian Edg7 receptor is a human Edg7 receptor.

This invention provides a method of screening a plurality of chemical compounds not known to inhibit the activation of a mammalian Edg7 receptor to identify a compound which inhibits the activation of the mammalian Edg7 receptor, which comprises: (a) contacting cells transfected with and expressing the mammalian Edg7 receptor with the plurality of compounds in the presence of a known mammalian Edg7 receptor agonist, under conditions permitting activation of the mammalian Edg7 receptor; (b) determining whether the activation of the mammalian Edg7 receptor is reduced in the presence of the plurality of compounds, relative to the activation of the mammalian Edg7 receptor in the absence of the plurality of compounds; and if so (c) separately determining the inhibition of activation of the mammalian Edg7 receptor for each compound included in the plurality of compounds, so as to thereby identify the compound which inhibits the activation of the mammalian Edg7 receptor. In one embodiment, the mammalian Edg7 receptor is a human Edg7 receptor. In another embodiment, wherein the cell is a mammalian cell. In another embodiment, the mammalian cell is non-neuronal in origin. In another embodiment, the non-neuronal cell is a COS-7 cell, a 293 human embryonic kidney cell, a LM(tk-) cell or an NIH-3T3 cell.

This invention provides a pharmaceutical composition comprising a compound identified by the method of this invention effective to increase mammalian Edg7 receptor activity and a pharmaceutically acceptable carrier.

This invention provides a pharmaceutical composition comprising a compound identified by the method of this invention effective to decrease mammalian Edg7 receptor activity and a pharmaceutically acceptable carrier.

This invention provides a method of treating an abnormality in a subject wherein the abnormality is alleviated by increasing the activity of a mammalian Edg7 receptor which comprises administering to the subject an amount of a compound which is a mammalian Edg7 receptor agonist effective to treat the abnormality. In one embodiment, the abnormality is a regulation of a steroid hormone disorder, an epinephrine release disorder, a gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a neuroendocrine disorder, a cognitive disorder, a memory disorder, a sensory modulation and transmission disorder, a motor coordination disorder, a sensory integration disorder, a motor integration disorder, a dopaminergic function disorder, an appetite disorder, obesity, a sensory transmission disorder, an olfaction disorder, a sympathetic innervation disorder, pain, psychotic behavior, affective disorder, migraine, cancer, proliferative diseases, wound healing, tissue regeneration, blood coagulation-related disorders, developmental disorders, or ischemia-reperfusion injury-related diseases.

This invention provides a method of treating an abnormality in a subject wherein the abnormality is alleviated by decreasing the activity of a mammalian Edg7 receptor which comprises administering to the subject an amount of a compound which is a mammalian Edg7 receptor antagonist effective to treat the abnormality. In one embodiment, the abnormality is a regulation of a steroid hormone disorder, an epinephrine release disorder, a gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a neuroendocrine disorder, a cognitive disorder, a memory disorder, a sensory modulation and transmission disorder, a motor coordination disorder, a sensory integration disorder, a motor integration disorder, a dopaminergic function disorder, an appetite disorder, obesity, a sensory transmission disorder, an olfaction disorder, a sympathetic innervation disorder, pain, psychotic behavior, affective disorder, migraine, cancer, proliferative diseases, wound healing, tissue regeneration, blood coagulation-related disorders, developmental disorders, or ischemia-reperfusion injury-related diseases.

This invention provides a process for making a composition of matter which specifically binds to a mammalian Edg7 receptor which comprises identifying a chemical compound using the process of this invention and then synthesizing the chemical compound or a novel structural and functional analog or homolog thereof. In one embodiment, the mammalian Edg7 receptor is a human Edg7 receptor.

This invention provides a process for preparing a pharmaceutical composition which comprises admixing a pharmaceutically acceptable carrier and a pharmaceutically acceptable amount of a chemical compound identified by the process of this invention or a novel structural and functional analog or homolog thereof. In one embodiment, the mammalian Edg7 receptor is a human Edg7 receptor.

Thus, once the gene for a targeted receptor subtype is cloned, it is placed into a recipient cell which then expresses the targeted receptor subtype on its surface. This cell, which expresses a single population of the targeted human receptor subtype, is then propagated resulting in the establishment of a cell line. This cell line, which constitutes a drug discovery system, is used in two different types of assays: binding assays and functional assays. In binding assays, the affinity of a compound for both the receptor subtype that is the target of a particular drug discovery program and other receptor subtypes that could be associated with side effects are measured. These measurements enable one to predict the potency of a compound, as well as the degree of selectivity that the compound has for the targeted receptor subtype over other receptor subtypes. The data obtained from binding assays also enable chemists to design compounds toward or away from one or more of the relevant subtypes, as appropriate, for optimal therapeutic efficacy. In functional assays, the nature of the response of the receptor subtype to the compound is determined. Data from the functional assays show whether the compound is acting to inhibit or enhance the activity of the receptor subtype, thus enabling pharmacologists to evaluate compounds rapidly at their ultimate human receptor subtypes targets permitting chemists to rationally design drugs that will be more effective and have fewer or substantially less severe side effects than existing drugs.

Approaches to designing and synthesizing receptor subtype-selective compounds are well known and include traditional medicinal chemistry and the newer technology of combinatorial chemistry, both of which are supported by computer-assisted molecular modeling. With such approaches, chemists and pharmacologists use their knowledge of the structures of the targeted receptor subtype and compounds determined to bind and/or activate or inhibit activation of the receptor subtype to design and synthesize structures that will have activity at these receptor subtypes.

Combinatorial chemistry involves automated synthesis of a variety of novel compounds by assembling them using different combinations of chemical building blocks. The use of combinatorial chemistry greatly accelerates the process of generating compounds. The resulting arrays of compounds are called libraries and are used to screen for compounds (“lead compounds”) that demonstrate a sufficient level of activity at receptors of interest. Using combinatorial chemistry it is possible to synthesize “focused” libraries of compounds anticipated to be highly biased toward the receptor target of interest.

Once lead compounds are identified, whether through the use of combinatorial chemistry or traditional medicinal chemistry or otherwise, a variety of homologs and analogs are prepared to facilitate an understanding of the relationship between chemical structure and biological or functional activity. These studies define structure activity relationships which are then used to design drugs with improved potency, selectivity and pharmacokinetic properties. Combinatorial chemistry is also used to rapidly generate a variety of structures for lead optimization. Traditional medicinal chemistry, which involves the synthesis of compounds one at a time, is also used for further refinement and to generate compounds not accessible by automated techniques. Once such drugs are defined the production is scaled up using standard chemical manufacturing methodologies utilized throughout the pharmaceutical and chemistry industry.

Finally, it is contemplated that this receptor will serve as a valuable tool for designing drugs for treating various pathophysiological conditions such as chronic and acute inflammation, arthritis, autoimmune diseases, transplant rejection, graft vs. host disease, bacterial, fungal, protozoan and viral infections, septicemia, AIDS, pain, psychotic and neurological disorders, including anxiety, depression, schizophrenia, dementia, mental retardation, memory loss, epilepsy, locomotor problems, respiratory disorders, asthma, eating/body weight disorders including obesity, bulimia, diabetes, anorexia, nausea, hypertension, hypotension, vascular and cardiovascular disorders, ischemia, stroke, cancers, ulcers, urinary retention, sexual/reproductive disorders, circadian rhythm disorders, renal disorders, bone diseases including osteoporosis, benign prostatic hypertrophy, gastrointestinal disorders, nasal congestion, allergies, Parkinson's disease, Alzheimer's disease, acute heart failure, angina disorders, delirium, dyskinesias such as Huntington's disease or Gilles dela Tourett's syndrome, among others and diagnostic assays for such conditions.

This invention will be better understood from the Experimental Details which follow. However, one skilled in the art will readily appreciate that the specific methods and results discussed are merely illustrative of the invention as described more fully in the claims which follow thereafter.

Experimental Details

Materials and Methods

MOPAC (Mixed Oligonucleotide Primed Amplification of cDNA)

100 ng of human genomic DNA (Clontech, Palo Alto, Calif.) was used for degenerate MOPAC PCR using Taq DNA polymerase (Boehringer-Mannheim, Indianapolis, Ind.) and the following degenerate oligonucleotides: JAB126, designed based on an alignment of the sixth transmembrane domain of several members of the rhodopsin superfamily of GPCRs; and JAB108, designed based on an alignment of the seventh transmembrane domain of the same rhodopsin superfamily.

The conditions for the MOPAC PCR reaction were as follows: 3 minute~hold at 94° C.; 10 cycles of 1 minute at 94° C., 1 minute 45 seconds at 44° C., 2 minutes at 72° C.; 30 cycles of 94° C. for 1 minute, 490 C. for 1 minute 45 seconds, 2 minutes at 72° C.; 4 minute hold at 72° C.; 4° C. hold until ready for agarose gel electrophoresis.

The products were run on a 1% agarose TAE gel and bands of the expected size (˜150 bp) were cut from the gel, purified using the QIAQUICK gel extraction kit (QIAGEN, Chatsworth, Calif.), and subcloned into the TA cloning vector (Invitrogen, San Diego, Calif.). White (insert-containing) colonies were picked and subjected to PCR using pCR2.1 vector primers JAB1 and JAB2 using the Expand Long Template PCR System and the following protocol: 94° C. hold for 3 minutes; 35 cycles of 94° C. for 1 minute, 68° C. for 1 minute 15 seconds; 2 minute hold at 68° C., 4° C. hold until the products were ready for purification. PCR products were purified by isopropanol precipitation (10 μl PCR product, 18 μl low TE, 10.5 μl 2M NaClO₄, and 21.5 μl isopropanol) and sequenced using the ABI Big Dye cycle sequencing protocol and ABI 377 sequencers (ABI, Foster City, Calif.). Nucleotide and amino acid sequence analyses were performed using the Wisconsin Package (GCG, Genetics Computer Group, Madison, Wis.) Two PCR products produced from human genoraic DNA (MPR3-HGEN-137 and MPR3-HGEN-152) were determined to be identical clones of a novel GPCR-like sequence based on database searches and its homology to other known GPCRs (78% DNA identity to Edg3, 73% DNA identity to Edg1 , 58% amino acid identity to Edg4, 48% amino acid identity to Edg3, and 47% amino acid identity to Edg2). This novel sequence was designated SNORF3.

Cloning of the full-length coding sequence of SNORF3

To determine the full-length coding sequence, we first performed 3′ Rapid Amplification of cDNA ends (RACE) using human pancreas Marathon-Ready cDNA (Clontech, Palo Alto, Calif.). 5 μl of the cDNA template was amplified with the Expand Long Template PCR System using the supplier's Adaptor Primer 1 and JAB222, a forward oligonucleotide 3′ to the sixth transmembrane domain. The conditions for this PCR were 1 minute at 94° C.; 5 cycles of 94° C. for 15 seconds and 72° C. for 1 minute 30 seconds; 5 cycles of 94° C. for 15 seconds and 70° C. for 1 minute 30 seconds; 22 cycles of 94° C. for 15 seconds and 68° C. for 1 minute 30 seconds; 68° C. hold for 5 minutes, and 4° C. hold until the products were ready for analysis. 1 μl of this reaction was subjected to a second round of PCR using the same conditions as the first round and the primers JAB229 and AP2. PCR products of approximately 500 bp and 800 bp were purified from a 1% agarose TAE gel, ligated into the TA Cloning Kit, and sequenced. Sequence analysis of products from each band indicated that both bands contained sequence overlapping the original SNORF3 clone and extending to a stop codon downstream from TM7.

From this sequence information, a new reverse 3′ primer, JAB302, was designed 3′ to the stop codon to be used with JAB229 for PCR screening of a human hippocampal cDNA library. Pools of a Synaptic Pharmaceutical Corporation human hippocampal cDNA library were screened by PCR with JAB229 and JAB302 and the Expand Long Template PCR system (Boehringer-Mannheim, Indianapolis, Ind.) with the following PCR protocol: 94° C. hold for 3 minutes; 40 cycles of 94° C. for 1 minute, 68° C. for 2 minutes; 4 minute hold at 68° C.; 4° C. hold until the samples are run on a gel. This screen yielded a positive pool #35 and a subsequent positive sub-pool #35-12. High stringency hybridization of isolated colonies from #35-12 with the SNORF3-specific oligonucleotide probe JAB255 and subsequent PCR testing of positive colonies indicated that the isolated clone #35-12-1 contained at least a partial clone of SNORF3. Sequencing of #35-12-1 revealed that this insert was 2.26 kb in length, containing the coding region of the receptor (1059 bp) plus 200 bp 5′ untranslated sequence and about 1 kb of 3′ untranslated sequence, in the mammalian expression vector pEXJ.BS. However, the SNORF3 insert was in the wrong orientation in this vector for expression, so this construct was digested with EcoRI and ligated into pcDNA3.1(−) which had been cut with EcoRI and treated with calf intestinal alkaline phosphatase (Boehringer-Mannheim). The new construct of SNORF3 in the correct orientation in pcDNA3.1(−) was named BN-10.

Oligonucleotide Primers

The following is a list of primers and their associated sequences which were used in the cloning of these receptors:

| | | |
|---|---|---|
| JAB126: | 5′-GYITWYRYIITIWSITGGHTICC-3′ | (SEQ ID NO:3) |
| JAB126: | 5′-G(T/C)IT(A/T)(T/C)(G/A)(T/C)IITI(A/T)(G/C)ITGG(A/C/T)TICC-3′ | (SEQ ID NO:3) |
| JAB108: | 5′- AVIADIGBRWAVAIIAIIGGRTT-3′ | (SEQ ID NO:4) |
| JAB108: | 5′-A(G/C/A)IA(G/A/T)IG(G/T/C)(G/A)(A/T)A(G/C/A)AIIAIIGG(G/A)TT-3′ | (SEQ ID NO:4) |
| JAB1: | 5′-TTATGCTTCCGGCTCGTATGTTGTG-3′ | (SEQ ID NO:5) |
| JAB2: | 5′-ATGTGCTGCAAGGCGATTAAGTTGGG-3′ | (SEQ ID NO:6) |
| JAB302: | 5′-ATCTATCTCGAGCCTGGGTGGGCCGAGAGGCATCC-3′ | (SEQ ID NO:7) |
| JAB229: | 5′-GCAGGCAGTGTGGCGTGCAGCATG-3′ | (SEQ ID NO:8) |
| JAB222: | 5′-TCTGCTCCTCGACGGCCTGAACTG-3′ | (SEQ ID NO:9) |
| JAB255: | 5′-GTGAAAAGGTGGTTCCTGCTGCTGGCGCTGCTCAACTCCGTCGTGAAC-3′ | (SEQ ID NO:10) |

Host Cells

A broad variety of host cells can be used to study heterologously expressed proteins. These cells include but are not limited to mammalian cell lines such as; Cos-7, CHO, LM(tk⁻), HEK293, etc.; insect cell lines such as; Sf9, Sf21, etc.; amphibian cells such as xenopus oocytes; assorted yeast strains; assorted bacterial cell strains; and others. Culture conditions for each of these cell types is specific and is known to those familiar with the art. The cells used to express Edg7 receptor were Cos-7, Human embryonic kidney (HEK) 293 and Chinese hamster ovary (CHO) cells.

COS-7 cells are grown on 150 mm plates in DMEM with supplements (Dulbecco's Modified Eagle Medium with 10% bovine calf serum, 4 mM glutamine, 100 units/ml penicillin/100 μg/ml streptomycin) at 37° C., 5% CO₂. Stock plates of COS-7 cells are trypsinized and split 1:6 every 3-4 days.

Human embryonic kidney 293 cells are grown on 150 mm plates in DMEM with supplements (10% bovine calf serum, 4 mM glutamine, 100 units/ml penicillin/100 μg/ml streptomycin) at 37° C., 5% CO₂. Stock plates of 293 cells are trypsinized and split 1:6 every 3-4 days.

CHO cells are grown on 150 mm plates in HAM's F-12 medium with supplements (10% bovine calf serum, 4 mM L-glutamine and 100 units/ml penicillin/ 100 μg/ml streptomycin) at 37° C., 5% CO₂. Stock plates of CHO cells are trypsinized and split 1:8 every 3-4 days.

Transient Expression

DNA encoding proteins to be studied can be transiently expressed in a variety of mammalian, insect, amphibian, yeast, bacterial and other cell lines by several transfection methods, including but not limited to, calcium phosphate-mediated, DEAE-dextran mediated, liposomal-mediated, viral-mediated, electroporation-mediated and microinjection delivery. Each of these methods may require optimization of assorted experimental parameters depending on the DNA, cell line, and the type of assay to be subsequently employed.

The electroporation method was used to transiently transfect various cell lines with Edg7 cDNA.

A typical protocol for the electroporation method as applied to Cos-7 cells is described as follows. Cells to be used for transfection are split 24 hours prior to the transfection to provide flasks which are subconfluent at the time of transfection. The cells are harvested by trypsinization resuspended in their growth media and counted. 5×10⁶ cells are suspended in 300 μl of DMEM and placed into an electroporation cuvette. 8 μg of receptor DNA plus 8 μg of any additional DNA needed (e.g. G protein expression vector, reporter construct, antibiotic resistance marker, mock vector, etc.) is added to the cell suspension, the cuvette is placed into a BioRad Gene Pulser and subjected to an electrical pulse (Gene Pulser settings: 0.25 kV voltage, 950 μF capacitance). Following the pulse, 800 μl of complete DMEM is added to each cuvette and the suspension transferred to a sterile tube. Complete medium is added to each tube to bring the final cell concentration to 1×10⁵ cells/100 μl. The cells are then plated as needed depending upon the type of assay to be performed.

Stable Expression

Heterologous DNA can be stably incorporated into host cells, causing the cell to perpetually express a foreign protein. Methods for the delivery of the DNA into the cell are similar to those described above for transient expression but require the co-transfection of an ancillary gene to confer drug resistance on the targeted host cell. The ensuing drug resistance can be exploited to select and maintain cells that have taken up the DNA. An assortment of resistance genes are available including but not restricted to neomycin, kanamycin, and hygromycin. For the purposes of studies concerning the receptor of this invention, stable expression of a heterologous receptor protein is typically carried out in, mammalian cells including but not necessarily restricted to, CHO, HEK293, LM(tk-), etc.

In addition native cell lines that naturally carry and express the nucleic acid sequences for the receptor may be used without the need to engineer the receptor complement.

Membrane Preparations

Cell membranes expressing the receptor protein of this invention are useful for certain types of assays including but not restricted to ligand binding assays, GTP-γ-S binding assays, and others. The specifics of preparing such cell membranes may in some cases be determined by the nature of the ensuing assay but typically involve harvesting whole cells and disrupting the cell pellet by sonication in ice cold buffer (e.g. 20 mM Tris-HCl, 5 mM EDTA, pH 7.4). The resulting crude cell lysate is cleared of cell debris by low speed centrifugation at 200×g for 5 min at 4° C. The cleared supernatant is then centrifuged at 40,000×g for 20 min at 4° C., and the resulting membrane pellet is washed by suspending in ice cold buffer and repeating the high speed centrifugation step. The final washed membrane pellet is resuspended in assay buffer. Protein concentrations are determined by the method of Bradford (1976) using bovine serum albumin as a standard. The membranes may be used immediately or frozen for later use.

Generation of Baculovirus

The coding region of DNA encoding the human receptor disclosed herein may be subcloned into pBlueBacIII into existing restriction sites or sites engineered into sequences 5′ and 3′ to the coding region of the polypeptides. To generate baculovirus, 0.5 μg of viral DNA (BaculoGold) and 3 μg of DNA construct encoding a polypeptide may be co-transfected into 2×10⁶ Spodoptera frugiperda insect Sf9 cells by the calcium phosphate co-precipitation method, as outlined by Pharmingen (in “Baculovirus Expression Vector System: Procedures and Methods Manual”). The cells then are incubated for 5 days at 27° C.

The supernatant of the co-transfection plate may be collected by centrifugation and the recombinant virus plaque purified. The procedure to infect cells with virus, to prepare stocks of virus and to titer the virus stocks are as described in Pharmingen's manual.

Labeled ligand Binding Assays

Cells expressing the receptor of this invention may be used to screen for ligands for said receptors, for example, by labeled ligand binding assays. Once a ligand is identified the same assays may be used to identify agonists or antagonists of the receptor that may be employed for a variety of therapeutic purposes.

In an embodiment, labeled ligands are placed in contact with either membrane preparations or intact cells expressing the receptor in multi-well microtiter plates, together with unlabeled compounds, and binding buffer. Binding reaction mixtures are incubated for times and temperatures determined to be optimal in separate equilibrium binding assays. The reaction is stopped by filtration through GF/B filters, using a cell harvester, or by directly measuring the bound ligand. If the ligand was labeled with a radioactive isotope such as ³H, ¹⁴C, ¹²⁵I, ³⁵S, ³²P, ³³P, etc., the bound ligand may be detected by using liquid scintillation counting, scintillation proximity, or any other method of detection for radioactive isotopes. If the ligand was labeled with a fluorescent compound, the bound labeled ligand may be measured by methods such as, but not restricted to, fluorescence intensity, time resolved fluorescence, fluorescence polarization, fluorescence transfer, or fluorescence correlation spectroscopy. In this manner agonist or antagonist compounds that bind to the receptor may be identified as they inhibit the binding of the labeled ligand to the membrane protein or intact cells expressing the said receptor. Non-specific binding is defined as the amount of labeled ligand remaining after incubation of membrane protein in the presence of a high concentration (e.g., 100-1000×K_{D}) of unlabeled ligand. In equilibrium saturation binding assays membrane preparations or intact cells transfected with the receptor are incubated in the presence of increasing concentrations of the labeled compound to determine the binding affinity of the labeled ligand. The binding affinities of unlabeled compounds may be determined in equilibrium competition binding assays, using a fixed concentration of labeled compound in the presence of varying concentrations of the displacing ligands.

Functional Assays

Cells expressing the Edg7 receptor DNA may be used to screen for ligands to Edg7 receptor using functional assays. Once a ligand is identified the same assays may be used to identify agonists or antagonists of the Edg7 receptor that may be employed for a variety of therapeutic purposes. It is well known to those in the art that the over-expression of a GPCR can result in the constitutive activation of intracellular signaling pathways. In the same manner, over-expression of the Edg7 receptor in any cell line as described above, can result in the activation of the functional responses described below, and any of the assays herein described can be used to screen for both agonist and antagonist ligands of the Edg7 receptor.

A wide spectrum of assays can be employed to screen for the presence of Edg7 receptor ligands. These assays range from traditional measurements of total inositol phosphate accumulation, cAMP levels, intracellular calcium mobilization, and potassium currents, for example; to systems measuring these same second messengers but which have been modified or adapted to be of higher throughput, more generic and more sensitive; to cell based assays reporting more general cellular events resulting from receptor activation such as metabolic changes, differentiation, cell division/proliferation. Description of several such assays follow.

Cyclic AMP (cAMP) Assay

The receptor-mediated stimulation or inhibition of cyclic AMP (cAMP) formation may be assayed in cells expressing the receptors. Cells are plated in 96-well plates or other vessels and preincubated in a buffer such as HEPES buffered saline (NaCl (150 mM), CaCl₂ (1 mM), KCl (5 mM), glucose (10 mM)) supplemented with a phosphodiesterase inhibitor such as 5 mM theophylline, with or without protease inhibitor cocktail (For example, a typical inhibitor cocktail contains 2 μg/ml aprotinin, 0.5 mg/ml leupeptin, and 10 μg/ml phosphoramidon.) for 20 min at 37° C., in 5% CO₂. Test compounds are added with or without 10 mM forskolin and incubated for an additional 10 min at 37° C. The medium is then aspirated and the reaction stopped by the addition of 100 mM HCl or other methods. The plates are stored at 4° C. for 15 min, and the cAMP content in the stopping solution is measured by radioimmunoassay.

Radioactivity may be quantified using a gamma counter equipped with data reduction software. Specific modifications may be performed to optimize the assay for the receptor or to alter the detection method of cAMP.

Arachidonic Acid Release Assay

Cells expressing the receptor are seeded into 96 well plates or other vessels and grown for 3 days in medium with supplements. ³H-arachidonic acid (specific activity=0.75 μCi/ml) is delivered as a 100 μL aliquot to each well and samples are incubated at 37° C., 5% CO₂ for 18 hours. The labeled cells are washed three times with medium. The wells are then filled with medium and the assay is initiated with the addition of test compounds or buffer in a total volume of 250 μL. Cells are incubated for 30 min at 37° C., 5% CO₂. Supernatants are transferred to a microtiter plate and evaporated to dryness at 75° C. in a vacuum oven. Samples are then dissolved and resuspended in 25 μL distilled water. Scintillant (300 μL) is added to each well and samples are counted for ³H in a Trilux plate reader. Data are analyzed using nonlinear regression and statistical techniques available in the GraphPAD Prism package (San Diego, Calif.).

Inositol Phosphate Assay

Edg7 receptor-mediated activation of the inositol phosphate (IP) second messenger pathways was assessed by radiometric measurement of IP products.

In a 96 well microplate format assay, cells are plated at a density of 70,000 cells per well and allowed to incubate for 24 hours. The cells are then labeled with 0.5 μCi [³H]myo-inositol overnight at 37° C., 5% CO_{2.} Immediately before the assay, the medium is removed and replaced with 90 μL of PBS containing 10 mM LiCl. The plates are then incubated for 15 min at 37° C., 5% CO₂. Following the incubation, the cells are challenged with agonist (10 μl/well; 10×concentration) for 30 min at 37° C., 5% CO₂. The challenge is terminated by the addition of 100 μL of 50% v/v trichloroacetic acid, followed by incubation at 4° C. for greater than 30 minutes. Total IPs are isolated from the lysate by ion exchange chromatography. Briefly, the lysed contents of the wells are transferred to a Multiscreen HV filter plate (Millipore) containing Dowex AG1-X8 (200-400 mesh, formate form). The filter plates are prepared adding 100μL of Dowex AG1-X8 suspension (50% v/v, water: resin) to each well. The filter plates are placed on a vacuum manifold to wash or elute the resin bed. Each well is first washed 2 times with 200 μl of 5 mM myo-inositol. Total [³H]inositol phosphates are eluted with 75 μl of 1.2M ammonium formate/0.1M formic acid solution into 96-well plates. 200 μL of scintillation cocktail is added to each well, and the radioactivity is determined by liquid scintillation counting.

Intracellular Calcium Mobilization Assays

The intracellular free calcium concentration may be measured by microspectrofluorimetry using the fluorescent indicator dye Fura-2/AM (Bush et al, 1991). Cells expressing the receptor are seeded onto a 35 mm culture dish containing a glass coverslip insert and allowed to adhere overnight. Cells are then washed with HBS and loaded with 100 μL of Fura-2/AM (10 μM) for 20 to 40 min. After washing with HBS to remove the Fura-2/AM solution, cells are equilibrated in HBS for 10 to 20 min. Cells are then visualized under the 40×objective of a Leitz Fluovert FS microscope and fluorescence emission is determined at 510 nM with excitation wavelengths alternating between 340 nM and 380 nM. Raw fluorescence data are converted to calcium concentrations using standard calcium concentration curves and software analysis techniques.

In another method, the measurement of intracellular calcium can also be performed on a 96-well (or higher) format and with alternative calcium-sensitive indicators, preferred examples of these are: aequorin, Fluo-3, Fluo-4, Fluo-5, Calcium Green-1, Oregon Green, and 488 BAPTA. After activation of the receptors with agonist ligands the emission elicited by the change of intracellular calcium concentration can be measured by a luminometer, or a fluorescence imager; a preferred example of this is the fluorescence imager plate reader (FLIPR).

Cells expressing the receptor of interest are plated into clear, flat-bottom, black-wall 96-well plates (Costar) at a density of 80,000-150,000 cells per well and allowed to incubate for 48 hr at 5% CO₂, 37° C. The growth medium is aspirated and 100 μl of loading medium containing fluo-3 dye is added to each well. The loading medium contains: Hank's BSS (without phenol red)(Gibco), 20 mM HEPES (Sigma), 0.1 or 1% BSA (Sigma), dye/pluronic acid mixture (e.g. 1 mM Flou-3, AM (Molecular Probes) and 10% pluronic acid (Molecular Probes) mixed immediately before use), and 2.5 mM probenecid (Sigma)(prepared fresh) The cells are allowed to incubate for about 1 hour at 5% CO₂, 37° C.

During the dye loading incubation the compound plate is prepared. The compounds are diluted in wash buffer (Hank's BSS (without phenol red), 20 mM HEPES, 2.5 mM probenecid) to a 4×final concentration and aliquoted into a clear v-bottom plate (Nunc). Following the incubation the cells are washed to remove the excess dye. A Denley plate washer is used to gently wash the cells 4 times and leave a 100 μl final volume of wash buffer in each well. The cell plate is placed in the center tray and the compound plate is placed in the right tray of the FLIPR. The FLIPR software is setup for the experiment, the experiment is run and the data are collected. The data are then analyzed using an excel spreadsheet program.

Antagonist ligands are identified by the inhibition of the signal elicited by agonist ligands.

GTPγS Functional Assay

Membranes from cells expressing the receptor are suspended in assay buffer (e.g., 50 mM Tris, 100 mM NaCl, 5 MM MgCl₂, 10 μM GDP, pH 7.4) with or without protease inhibitors (e.g., 0.1% bacitracin). Membranes are incubated on ice for 20 minutes, transferred to a 96-well Millipore microtiter GF/C filter plate and mixed with GTPγ³⁵S (e.g., 250,000 cpm/sample, specific activity ˜1000 Ci/mmol) plus or minus unlabeled GTPγS (final concentration=100 μM). Final membrane protein concentration=90 μg/ml. Samples are incubated in the presence or absence of test compounds for 30 min. at room temperature, then filtered on a Millipore vacuum manifold and washed three times with cold (4° C.) assay buffer. Samples collected in the filter plate are treated with scintillant and counted for ³⁵S in a Trilux (Wallac) liquid scintillation counter. It is expected that optimal results are obtained when the receptor membrane preparation is derived from an appropriately engineered heterologous expression system, i.e., an expression system resulting in high levels of expression of the receptor and/or expressing G-proteins having high turnover rates (for the exchange of GDP for GTP). GTPγS assays are well-known to those skilled in the art, and it is contemplated that variations on the method described above, such as are described by Tian et al. (1994) or Lazareno and Birdsall (1993), may be used.

Microphysiometric Assay

Because cellular metabolism is intricately involved in a broad range of cellular events (including receptor activation of multiple messenger pathways), the use of microphysiometric measurements of cell metabolism can in principle provide a generic assay of cellular activity arising from the activation of any receptor regardless of the specifics of the receptor's signaling pathway.

General guidelines for transient receptor expression, cell preparation and microphysiometric recording are described elsewhere (Salon, J. A. and Owicki, J. A., 1996). Typically cells expressing receptors are harvested and seeded at 3×10⁵ cells per microphysiometer capsule in complete media 24 hours prior to an experiment. The media is replaced with serum free media 16 hours prior to recording to minimize non-specific metabolic stimulation by assorted and ill-defined serum factors. On the day of the experiment the cell capsules are transferred to the microphysiometer and allowed to equilibrate in recording media (low buffer RPMI 1640, no bicarbonate, no serum (Molecular Devices Corporation, Sunnyvale, Calif.) containing 0.1% fatty acid free BSA), during which a baseline measurement of basal metabolic activity is established.

A standard recording protocol specifies a 100 μl/min flow rate, with a 2 min total pump cycle which includes a 30 sec flow interruption during which the acidification rate measurement is taken. Ligand challenges involve a 1 min 20 sec exposure to the sample just prior to the first post challenge rate measurement being taken, followed by two additional pump cycles for a total of 5 min 20 sec sample exposure. Typically, drugs in a primary screen are presented to the cells at 10 μM final concentration. Follow up experiments to examine dose-dependency of active compounds are then done by sequentially challenging the cells with a drug concentration range that exceeds the amount needed to generate responses ranging from threshold to maximal levels. Ligand samples are then washed out and the acidification rates reported are expressed as a percentage increase of the peak response over the baseline rate observed just prior to challenge.

MAP Kinase Assay

MAP kinase (mitogen activated kinase) may be monitored to evaluate receptor activation. MAP kinase is activated by multiple pathways in the cell. A primary mode of activation involves the ras/raf/MEK/MAP kinase pathway. Growth factor (tyrosine kinase) receptors feed into this pathway via SHC/Grb-2/SOS/ras. Gi coupled receptors are also known to activate ras and subsequently produce an activation of MAP kinase. Receptors that activate phospholipase C (such as Gq/G11-coupled) produce diacylglycerol (DAG) as a consequence of phosphatidyl inositol hydrolysis. DAG activates protein kinase C which in turn phosphorylates MAP kinase.

MAP kinase activation can be detected by several approaches. One approach is based on an evaluation of the phosphorylation state, either unphosphorylated (inactive) or phosphorylated (active). The phosphorylated protein has a slower mobility in SDS-PAGE and can therefore be compared with the unstimulated protein using Western blotting. Alternatively, antibodies specific for the phosphorylated protein are available (New England Biolabs) which can be used to detect an increase in the phosphorylated kinase. In either method, cells are stimulated with the test compound and then extracted with Laemmli buffer. The soluble fraction is applied to an SDS-PAGE gel and proteins are transferred electrophoretically to nitrocellulose or Immobilon. Immunoreactive bands are detected by standard Western blotting technique. Visible or chemiluminescent signals are recorded on film and may be quantified by densitometry.

Another approach is based on evaluation of the MAP kinase activity via a phosphorylation assay. Cells are stimulated with the test compound and a soluble extract is prepared. The extract is incubated at 30° C. for 10 min with gamma-³²P-ATP, an ATP regenerating system, and a specific substrate for MAP kinase such as phosphorylated heat and acid stable protein regulated by insulin, or PHAS-I. The reaction is terminated by the addition of H₃PO₄ and samples are transferred to ice. An aliquot is spotted onto Whatman P81 chromatography paper, which retains the phosphorylated protein. The chromatography paper is washed and counted for ³²P in a liquid scintillation counter. Alternatively, the cell extract is incubated with gamma-³²P-ATP, an ATP regenerating system, and biotinylated myelin basic protein bound by streptavidin to a filter support. The myelin basic protein is a substrate for activated MAP kinase. The phosphorylation reaction is carried out for 10 min at 30° C. The extract can then by aspirated through the filter, which retains the phosphorylated myelin basic protein. The filter is washed and counted for ³²P by liquid scintillation counting.

Cell Proliferation Assay

Receptor activation of the receptor may lead to a mitogenic or proliferative response which can be monitored via ³H-thymidine uptake. When cultured cells are incubated with ³H-thymidine, the thymidine translocates into the nuclei where it is phosphorylated to thymidine triphosphate. The nucleotide triphosphate is then incorporated into the cellular DNA at a rate that is proportional to the rate of cell growth. Typically, cells are grown in culture for 1-3 days. Cells are forced into quiescence by the removal of serum for 24 hrs. A mitogenic agent is then added to the media. 24 hrs later, the cells are incubated with ³H-thymidine at specific activities ranging from 1 to 10 uCi/ml for 2-6 hrs. Harvesting procedures may involve trypsinization and trapping of cells by filtration over GF/C filters with or without a prior incubation in TCA to extract soluble thymidine. The filters are processed with scintillant and counted for ³H by liquid scintillation counting. Alternatively, adherent cells are fixed in MeOH or TCA, washed in water, and solubilized in 0.05% deoxycholate/0.1 N NaOH. The soluble extract is transferred to scintillation vials and counted for ³H by liquid scintillation counting.

Alternatively, cell proliferation can be assayed by measuring the expression of an endogenous or heterologous gene product, expressed by the cell line used to transfect the receptor, which can be detected by methods such as, but not limited to, florescence intensity, enzymatic activity, immunoreactivity, DNA hybridization, polymerase chain reaction, etc.

Promiscuous Second Messenger Assays

It is not possible to predict, a priori and based solely upon the GPCR sequence, which of the cell's many different signaling pathways any given receptor will naturally use. It is possible, however, to coax receptors of different functional classes to signal through a pre-selected pathway through the use of promiscuous G_{α} subunits. For example, by providing a cell based receptor assay system with an endogenously supplied promiscuous G_{α} subunit such as G_{α15} or G_{α16} or a chimeric G_{α} subunit such as G_{αqz}, a GPCR, which might normally prefer to couple through a specific signaling pathway (e.g., Gₛ, Gₗ, G_{q}, G₀, etc.), can be made to couple through the pathway defined by the promiscuous G_{α} subunit and upon agonist activation produce the second messenger associated with that subunit's pathway. In the case of G_{α15}, G_{α16} and/or G_{αqz} this would involve activation of the G_{q} pathway and production of the second messenger IP₃. Through the use of similar strategies and tools, it is possible to bias receptor signaling through pathways producing other second messengers such as Ca⁺⁺, cAMP, and K⁺ currents, for example (Milligan, 1999).

It follows that the promiscuous interaction of the exogenously supplied G_{α} subunit with the receptor alleviates the need to carry out a different assay for each possible signaling pathway and increases the chances of detecting a functional signal upon receptor activation.

Methods for Recording Currents in Xenopus Oocytes

Oocytes are harvested from Xenopus laevis and injected with mRNA transcripts as previously described (Quick and Lester, 1994; Smith et al.,1997). Edg7 receptor RNA transcript is synthesized using the T7 polymerase (“Message Machine,” Ambion) from linearized plasmids or PCR products containing the complete coding region of the gene. Oocytes are injected with 1-50 ng synthetic receptor RNA and incubated for 3-8 days at 17° C. Currents are recorded under dual electrode voltage clamp (Axon Instruments Inc.) with 3 M KCl-filled glass microelectrodes having resistances of 1-2 Mohm. Unless otherwise specified, oocytes are voltage clamped at a holding potential of −80 mV. During recordings, oocytes are bathed in continuously flowing (1-3 ml/min) medium containing 96 mM NaCl, 2 mM KCl, 1.8 MM CaCl₂, 1 mM MgCl₂, and 5 mM HEPES, pH 7.5 (ND96). Drugs are applied either by local perfusion from a 10 μl glass capillary tube fixed at a distance of 0.5 mm from the oocyte, or by switching from a series of gravity fed perfusion lines.

Other oocytes may be injected with a mixture of receptor mRNAs and synthetic mRNA encoding the genes for G-protein-activated inward rectifier channels (GIRK1 and GIRK4, U.S. Pat. Nos. 5,734,021 and 5,728,535 or GIRK1 and GIRK2) or any other appropriate combinations (see, e.g., Inanobe et al., 1999). Genes encoding G-protein inwardly rectifying K⁺ (GIRK) channels 1, 2 and 4 (GIRK1, GIRK2, and GIRK4) may be obtained by PCR using the published sequences (Kubo et al., 1993; Dascal et al., 1993; Krapivinsky et al., 1995 and 1995b) to derive appropriate 5′ and 3′ primers. Human heart or brain cDNA may be used as template together with appropriate primers.

Heterologous expression of GPCRs in Xenopus oocytes has been widely used to determine the identity of signaling pathways activated by agonist stimulation (Gundersen et al., 1983; Takahashi et al., 1987).

Activation of the phospholipase C (PLC) pathway is assayed by applying a test compound in ND96 solution to oocytes previously injected with mRNA for the Edg7 receptor and observing inward currents at a holding potential of approximately −80 mV. The appearance of currents that reverse at −25 mV and display other properties of the Ca⁺⁺-activated Cl⁻ channel is indicative of receptor-activation of PLC and release of IP₃ and intracellular Ca⁺⁺. Such activity is exhibited by GPCRs that couple to G_{q} or G₁₁.

Involvement of the G_{i/o} class of G-proteins in GPCR-stimulated Ca⁺⁺-activated Cl⁻ currents is evaluated using PTX, a toxin which inactivates G_{i/o} G-proteins. Oocytes are injected with 25 ng PTX/oocyte and modulation of Ca⁺⁺-activated Cl⁻ currents by Edg7 receptor is evaluated 2-5 h subsequently.

Measurement of inwardly rectifying K⁺ (potassium) channel (GIRK) activity may be monitored in oocytes that have been co-injected with mRNAs encoding the mammalian receptor plus GIRK subunits. GIRK gene products co-assemble to form a G-protein activated potassium channel known to be activated (i.e., stimulated) by a number of GPCRs that couple to Gⁱ or Gₒ (Kubo et al., 1993; Dascal et al., 1993). Oocytes expressing the mammalian receptor plus the GIRK subunits are tested for test compound responsivity by measuring K⁺ currents in elevated K⁺ solution containing 49 mM K⁺.

Localization of mRNA Coding for Human Edg7 Receptors

Development of probes for Edg7: To facilitate the production of radiolabeled, antisense RNA probes a fragment of the gene encoding human Edg7 was subcloned into a plasmid vector containing RNA polymerase promoter sites. The full-length CDNA encoding the Edg7 receptor was used as a template for PCR amplification of a fragment of the coding sequence. The following synthetic oligonucleotide primers were used:

| | | |
|---|---|---|
| Forward primer | | |
| SN3-F1/ EcoRI | 5′- GTCACGAATTCGTCATGACACTGCTCATTTTGCT-3′ | (SEQ ID NO:11) |
| Reverse primer | | |
| SN3-B1/BamH | 5′-GTCACGGATCCGTCATGTCATCACCGTCTTCAT T-3′ | (SEQ ID NO:12) |

The oligonucleotides used will amplify a 296 base pair fragment of the Edg7 gene. They contain a fragment of the Edg7 gene as well as restriction endonuclease recognition sequences to facilitate cloning. PCR was carried out using Expand High Fidelity reagents (Boehringer Mannheim) using standard protocols supplied with the PCR reagents. Following PCR, the amplified DNA was size fractionated on an agarose gel, purified, and digested with EcoRI and BamHI. This fragment was cloned into the EcoRI and BamHI sites of pGEM 3 z (Promega, Madison Wis.), containing both sp6 and T7 RNA polymerase promoter sites. The construct was sequenced to confirm sequence identity and orientation. To synthesize antisense strands of RNA, this construct was linearized with EcoRI and sp6 RNA polymerase was used to incorporate radiolabeled nucleotide as described below.

A probe coding for the rat glyceraldehyde 3-phosphate dehydrogenase (GAPDH) gene, a constitutively expressed protein, was used concurrently. GAPDH is expressed at a relatively constant level in most tissue and its detection is used to compare expression levels of the rat Edg7 receptor gene in different regions.

Synthesis of probes: Edg7 and GAPDH cDNA sequences preceded by phage polymerase promoter sequences were used to synthesize radiolabeled riboprobes. Conditions for the synthesis of riboprobes were: 0.25-1.0 μg linearized DNA plasmid template, 1.5 μl of ATP, GTP, UTP (10 mM each), 3 μl dithiothreitol (0.1 M), 30 units RNAsin RNAse inhibitor, 0.5-1.0 μl (15-20 units/μl) RNA polymerase, 7.0 μl transcription buffer (Promega Corp.), and 12.5 μl alpha ³²P-CTP (specific activity 3,000 Ci/mmol). 0.1 mM CTP (0.02-1.0 μl) was added to each reaction, and the volume was adjusted to 35 μl with DEPC-treated water. Labeling reactions were incubated at 37° C. for 60 min, after which 3 units of RQ1 RNAse-free DNAse (Promega Corp.) was added to digest the template. Riboprobes were separated from unincorporated nucleotides using Microspin S-300 columns (Pharmacia Biotech). TCA precipitation and liquid scintillation spectrometry were used to measure the amount of label incorporated into the probe. A fraction of all riboprobes synthesized were size-fractionated on 0.25 mm thick 7M urea, 4.5% acrylamide sequencing gels. These gels were apposed to storage phosphor screens and the resulting autoradiograph scanned using a phoshorimager (Molecular Dynamics, Sunnyvale, Calif.) to confirm that the probes synthesized were full-length and not degraded.

Solution hybridization/ribonuclease protection assay (RPA): For solution hybridization 2.0 μg of mRNA isolated from tissues were used. Negative controls consisted of 30 μg transfer RNA (tRNA) or no tissue blanks. All mRNA samples were placed in 1.5-ml microfuge tubes and vacuum dried. Hybridization buffer (40 μl of 400 mM NaCl, 20 mM Tris, pH 6.4, 2 mM EDTA, in 80% formamide) containing 0.25-2.0 E⁶ counts of each probe was added to each tube. Samples were heated at 95° C. for 15 min, after which the temperature was lowered to 55° C. for hybridization.

After hybridization for 14-18 hr, the RNA/probe mixtures were digested with RNAse A (Sigma) and RNAse T1 (Life Technologies). A mixture of 2.0 μg RNAse A and 1000 units of RNAse T1 in a buffer containing 330 mM NaCl, 10 mM Tris (pH 8.0) and 5 mM EDTA (400 μl) were added to each sample and incubated for 90 min at room temperature. After digestion with RNAses, 20 μl of 10% SDS and 50 μg proteinase K was added to each tube and incubated at 37° C. for 15 min. Samples were extracted with phenol/chloroform:isoamyl alcohol and precipitated in 2 volumes of ethanol for 1 hr at −70° C. Pellet Paint (Novagen) was added to each tube (2.0 μg) as a carrier to facilitate precipitation. Following precipitation, samples were centrifuged, washed with cold 70% ethanol, and vacuum dried. Samples were dissolved in formamide loading buffer and size-fractionated on a urea/acrylamide sequencing gel (7.0 M urea, 4.5% acrylamide in Tris-borate-EDTA). Gels were dried and apposed to storage phosphor screens and scanned using a phosphorimager (Molecular Dynamics, Sunnyvale, Calif.).

RT-PCR: For the detection of RNA encoding human Edg7, RT-PCR was carried out on mRNA extracted from human tissue. Reverse transcription and PCR reactions were carried out in 50 μl volumes using EZrTth DNA polymerase (Perkin Elmer). Primers with the following sequences were used:

| | |
|---|---|
| Forward primer | |
| SN3-F | |
| 5′-TCTCTGCCTGCTCTTCCCT-3′ | (SEQ ID NO:13) |
| Reverse primer | |
| SN3-B | |
| 5′-CATACCACAAACGCCCCT-3′ | (SEQ ID NO:14) |

These primers will amplify a 234 base pair fragment from nucleotides 544 to 778 of the sequence shown in FIGS. 1A-1B.

Each reaction contained 0.1 μg mRNA and 0.3 μM of each primer. Concentrations of reagents in each reaction were: 300 μM each of dGTP; DATP; dCTP; dTTP; 2.5 mM Mn(OAc)_{2 ; 50} mM Bicine; 115 mM potassium acetate, 8% glycerol and 5 units EZrTth DNA polymerase. All reagents for PCR (except mRNA and oligonucleotide primers) were obtained from Perkin Elmer. Reactions were carried out under the following conditions: 65° C. 60 min., 94° C. 2 min., (94° C., 1 min., 65° C. 1 min) 35 cycles, 72° C. 10 min. PCR reactions were size fractionated by gel electrophoresis using 10% polyacrylamide. DNA was stained with SYBR Green I (Molecular Probes, Eugene Oreg.) and scanned on a Molecular Dynamics (Sunnyvale Calif.) Storm 860 in blue fluorescence mode at 450 nM.

Positive controls for PCR reactions consisted of amplification of the target sequence from a plasmid construct, as well as reverse transcribing and amplifying a known sequence. Negative controls consisted of mRNA blanks, as well as primer and mRNA blanks. To confirm that the mRNA was not contaminated with genomic DNA, samples were digested with RNAses before reverse transcription. Integrity of RNA was assessed by amplification of mRNA coding for GAPDH.

Results and Discussion

Cloning of the Full-Length Sequence of Edg7

100 ng human genomic DNA was subjected to MOPAC PCR with two degenerate primers designed based on the sixth and seventh transmembrane domains of receptors from the rhodopsin superfamily of GPCRs. Two products from this reaction, MPR3-HGEN-137 and MPR3-HGEN-152 were found to be identical clones of a novel DNA sequence not found in the Genbank databases (Genembl, STS, EST, or GSS), which had a high degree of identity to the Edg family of GPCRs (78% DNA identity to Edg3, 73% DNA identity to Edg1, 58% amino acid identity to Edg4, 48% amino acid identity to Edg3, and 47% amino acid identity to Edg2). This novel partial clone was given the name SNORF3.

The full-length SNORF3 sequence was acquired by using 3′ RACE followed by PCR screening of pools of a human hippocampal cDNA library with specific SNORF3 oligonucleotide primers. This library screen yielded a positive pool #35 and a subsequent positive sub-pool #35-12. High stringency hybridization of isolated colonies from #35-12 with the SNORF3-specific oligonucleotide probe JAB255 and subsequent PCR testing of positive colonies indicated that the isolated clone #35-12-1 contained at least a partial clone of SNORF3. Sequencing of #35-12-1 revealed that the insert was 2.26 kb in length, containing the coding region of the receptor (1059 bp) plus 200 bp 5′ untranslated sequence and about 1 kb of 3′ untranslated sequence, in the mammalian expression vector pEXJ.BS. This insert was in the wrong orientation for expression, so this construct was digested with EcoRI and ligated in the correct orientation into pcDNA3.1 (−). The new construct of SNORF3 in pcDNA3.1 (−) was named BN-10. The nucleotide sequence of SNORF3 is represented in FIGS. 1A-1B. As shown in FIGS. 1A-1B, SNORF3 contains two potential initiating methionine (ATG) codons upstream of TMI at positions 27-29 and 54-56 and a stop codon at positions 1086-1088. FIGS. 2A-2B shows the translated amino acid sequence of the longest open reading frame of SNORF3 indicated in FIGS. 1A-1B.

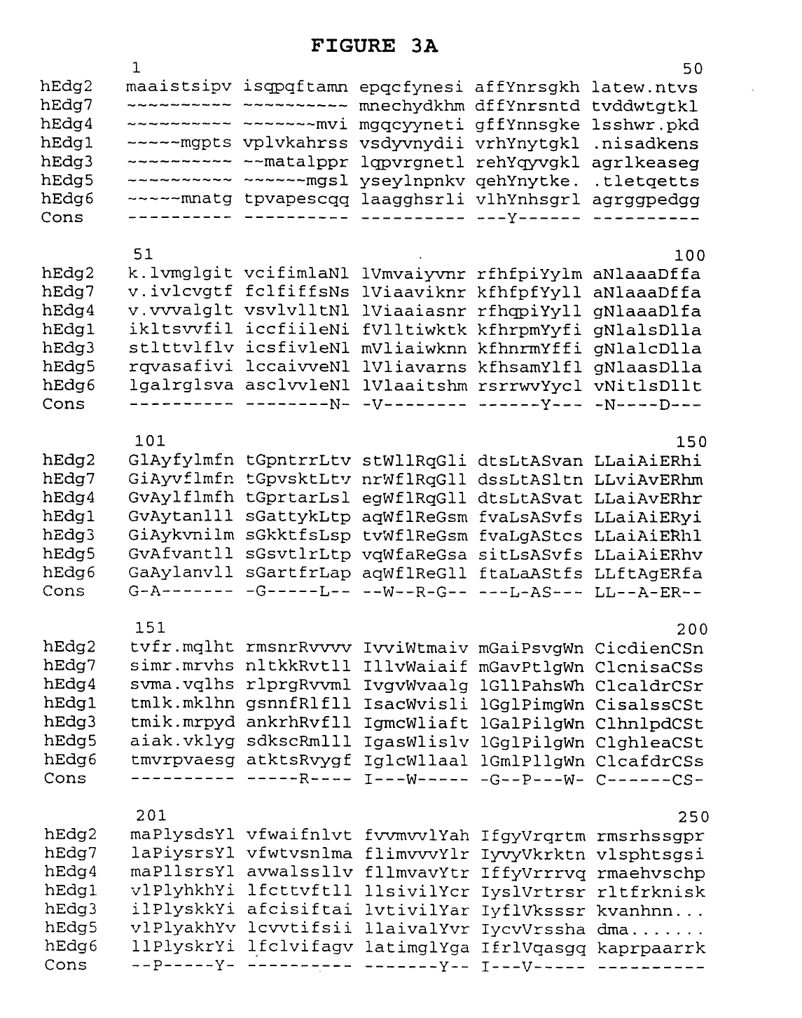

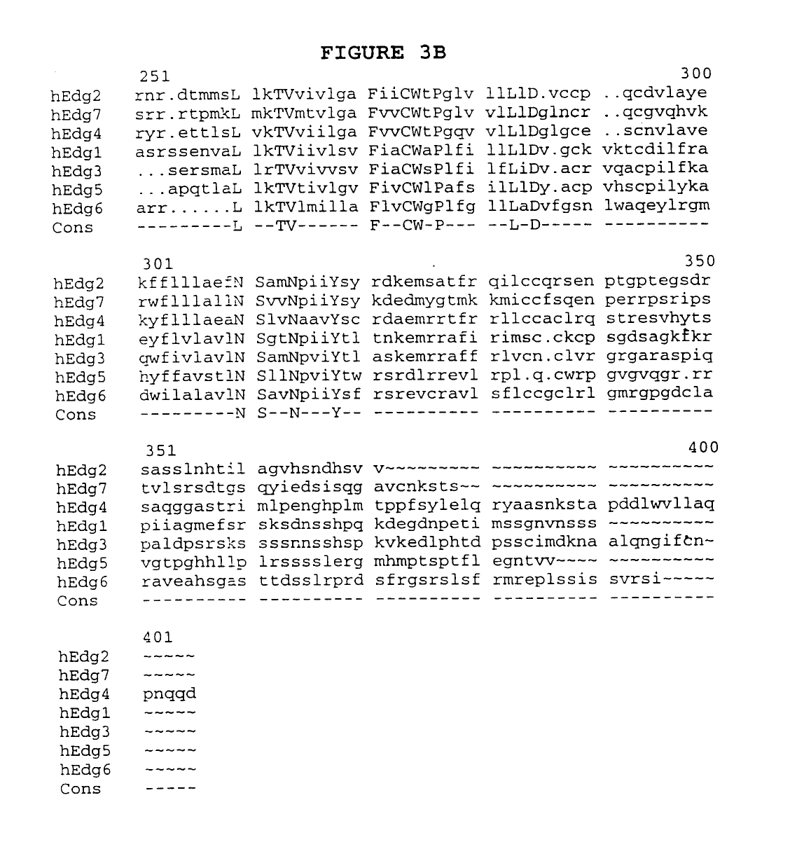

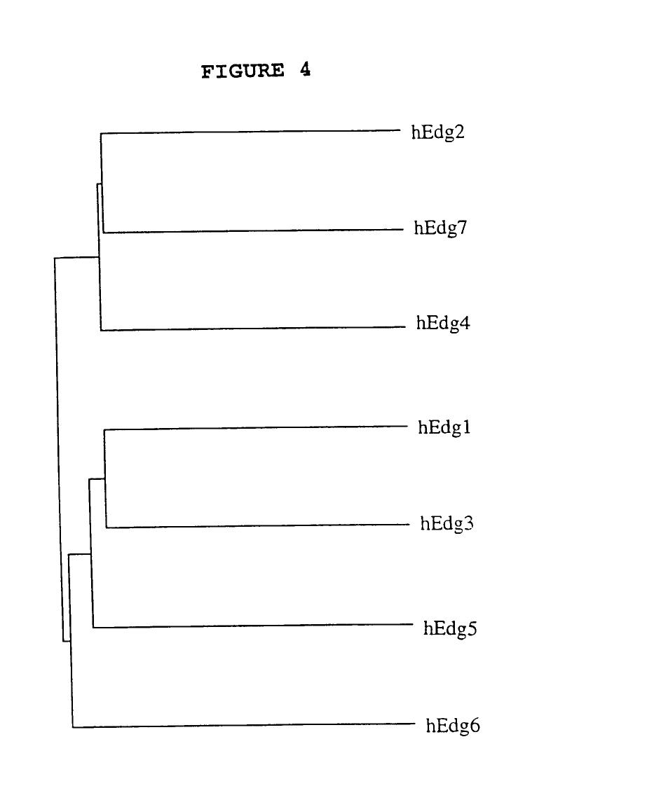

Hydophobicity (Kyte-Doolittle) analysis of the amino acid sequence of the full-length clone indicates the presence of seven hydrophobic regions, which is consistent with the seven transmembrane domains of a G protein-coupled receptor. The seven expected transmembrane domains are mapped out in FIGS. 2A-2B. A comparison of nucleotide and peptide sequences of SNORF3 with sequences contained in the Genbank/EMBL/SwissProtPlus databases reveals that the, amino acid sequence of this clone is most related to the Endothelial Differentiation Gene (Edg) family of GPCRs, with 49% amino acid identity to hEdg2 (Q92633), 46% identity to hEdg4 (AF011466), and 30-33% identity to hEdg1 (P21453), hEdg3 (Q99500), hEdg5 (AF034780), and hEdg6 (AJ000479). Similarly, the percent nucleotide identity of SNORF3 was highest to Edg2 (58%, Y09479) and Edg4 (54%, AF011466), indicating that it was likely to be a member of the Edg subfamily of GPCRs. Due to the high degree of homology of SNORF3 with members of the Edg family, we have renamed SNORF3 as Edg7. An amino acid alignment of Edg7 with other members of the Edg family is shown in FIG. 3. A dendrogram shown in FIG. 4 demonstrates that Edg7 and Edg2 cluster closer to each other than to any of the other Edg family members.

Edg7 possesses several potential protein kinase C (PKC) phosphorylation motifs throughout its amino acid sequence: threonine 132 in the second intracellular loop, serine 220 in the third intracellular loop, serine 285 at the c-terminal end of TMVII, and threonine 294 in the C-terminal tail. Serines 285 and 320 are potential casein kinase 2 phosphorylation sites, while threonines 208 and 224 in the third intracellular loop, as well as serine 312 in the C-terminal tail, are potential cAMP-dependent protein kinase phosphorylation sites. There are also two potential N-linked glycosylation sites at asparagine 6 in the N-terminal tail and at asparagine 163 in the second extracellular loop.

Inositol Phosphate Response of Edg7-Transfected Cells

The expression vector (pcDNA) containing the Edg7 cDNA was transfected by electroporation into Cos-7 cells. After plating and labeling with [³H]myo-inositol, the transfectants were challenged with a ligand library that included, among other things, oleoyl lysophosphatidic acid (oleoyl LPA) (1 μM final concentration) and then assayed for inositol phosphate (IP) formation. When challenged with oleoyl LPA, the most active LPA analogue at LPA receptors (Jalink et al., 1995), cells transfected with Edg7 gave an average 3-fold increase in IP production as compared to mock (empty vector)—transfected cells which gave an average 2-fold increase (n=4).

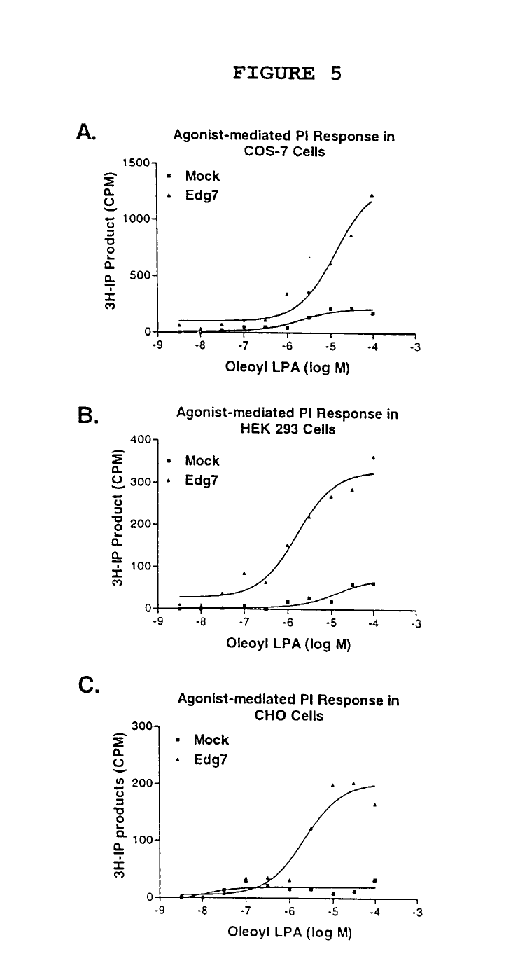

Subsequent experiments demonstrated that the oleoyl LPA-induced increase in IP formation was independent of host cell as it was observed also in HEK 293 and CHO cells (n=2) (FIG. 5). The IP response to oleoyl LPA in all three cell lines was concentration-dependent with EC₅₀ values ranging from 1 to 10 μM and Eₘₐₓ of approximately 200-300% (FIG. 5).

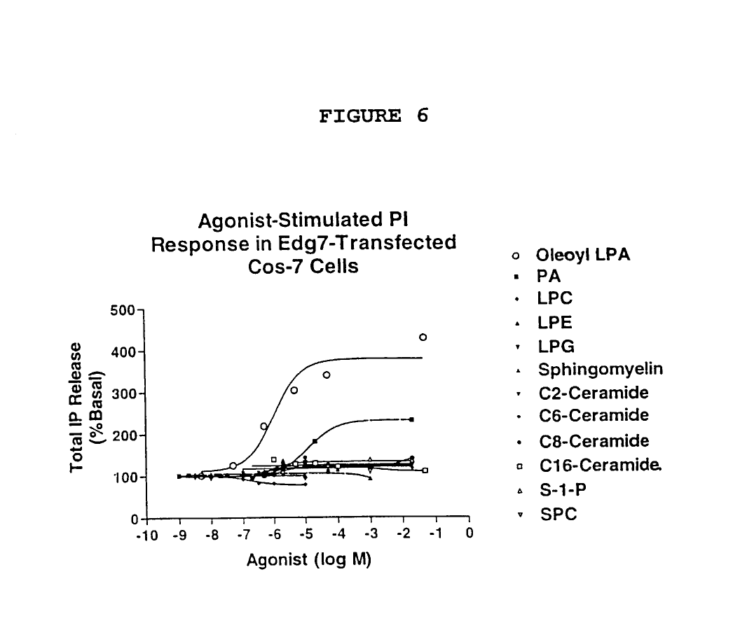

Several additional phospholipids, including sphingosine-1-phosphate, were tested for their ability to activate Edg7. No dose-responsiveness of inositol phosphate formation could be detected in Cos-7 cells transfected with Edg7 when challenged with several lysophosphatides, ceramides, sphingosine-1-phosphate or sphingosylphosphorylcholine (FIG. 6). In contrast, phosphatidic acid was able to activate Edg7 in a concentration-dependent fashion and behaved as a partial agonist with respect to oleoyl LPA (FIG. 6).

Increase in Intracellular Calcium in Edg7-Transfected Cells

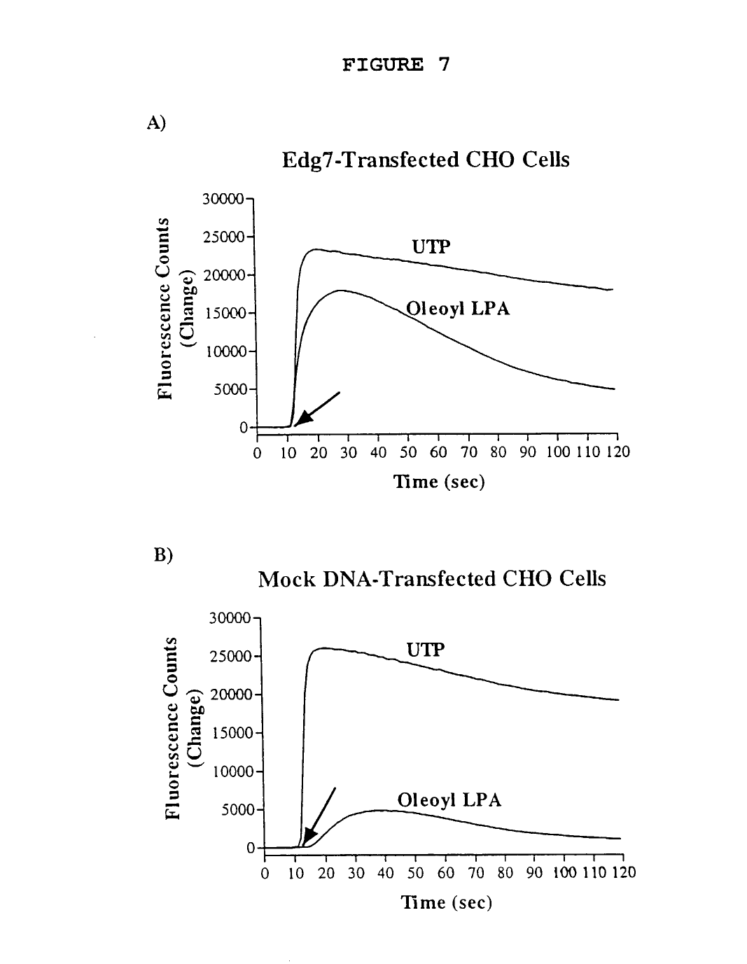

Enhancement of intracellular calcium is almost a ubiquitous response to LPA. To examine whether activation of Edg7 receptors by oleoyl LPA would induce the response, an intracellular calcium mobilization assay was performed in Edg7-transfected and mock DNA (empty vector)-transfected CHO cells. As shown in FIG. 7, oleoyl LPA (10 μM) increased intracellular calcium in both mock DNA- and Edg7-transfected cells. However, the peak response was approximately 3-fold higher in Edg7-transfected cells as compared to the response in mock DNA-transfected cells, suggesting that activation of Edg7 receptors by oleoyl LPA indeed resulted in an increase in intracellular calcium.

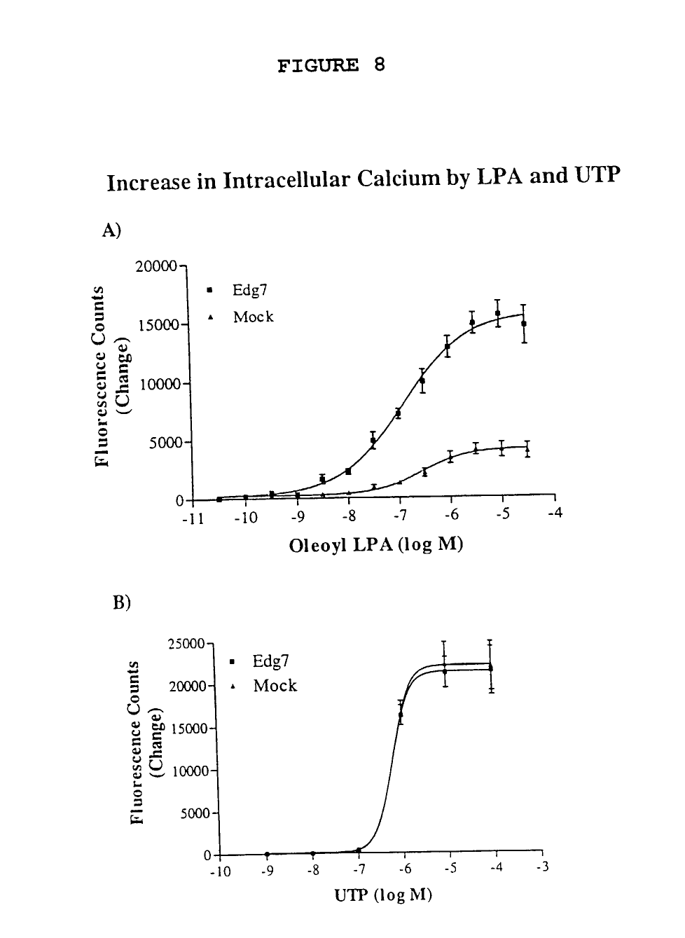

Further evaluation revealed that the oleoyl LPA-mediated increase in intracellular calcium was concentration-dependent both in Edg7- and mock DNA-transfected cells with EC₅₀ values of 136±19 nM and 347±54 nM, respectively (FIG. 8A). In contrast, responses to UTP in both the cell lines were almost identical (FIG. 8B), suggesting that the enhanced maximal response to oleoyl LPA observed in Edg7-expressing cells, as compared to mock DNA-transfected cells, was not due to a change in cell density or in the intrinsic properties of the cells.

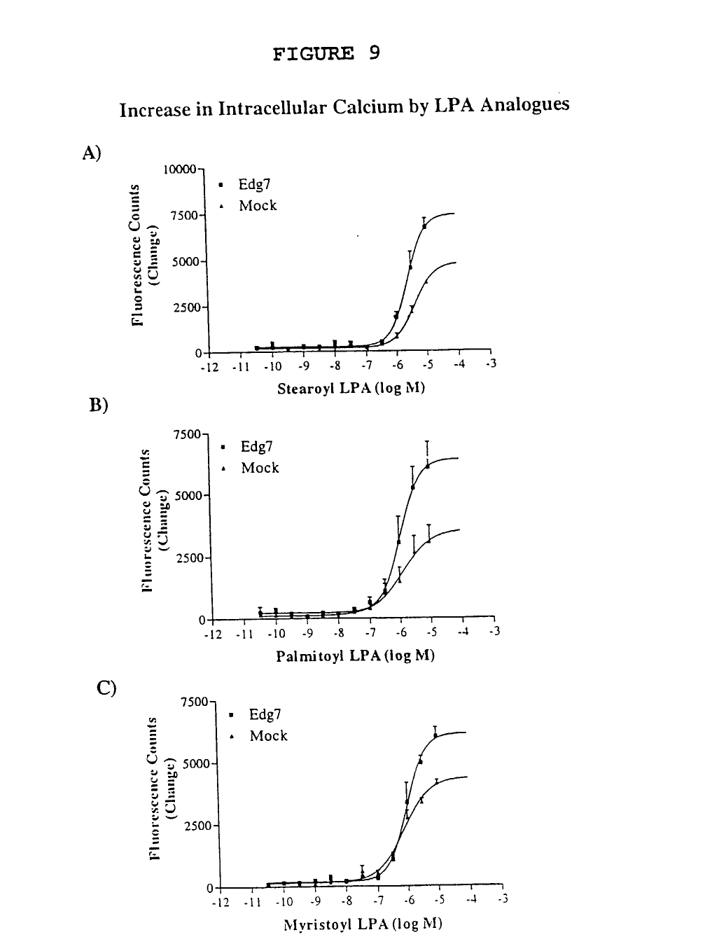

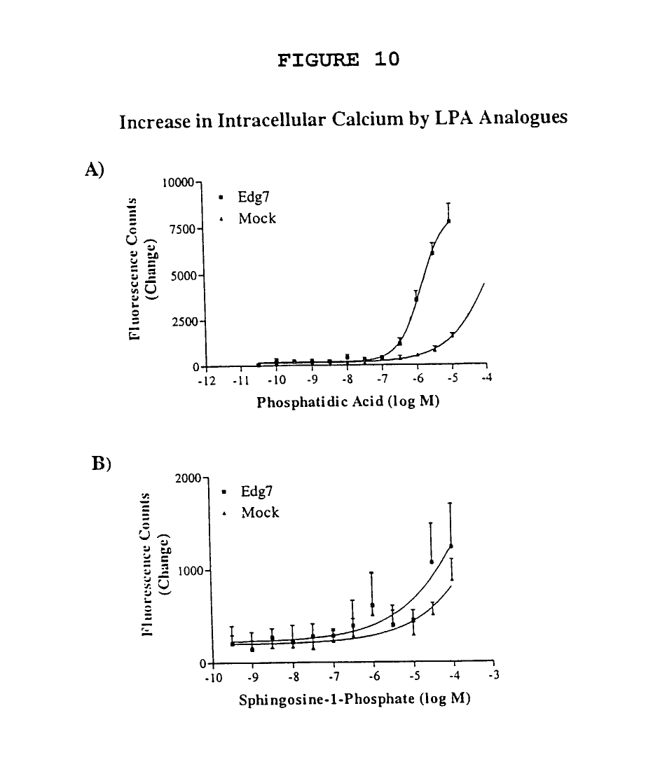

It has been reported in the literature that LPA analogues with different acyl chain lengths can activate LPA receptors. In accordance with this observation, stearoyl LPA, palmitoyl LPA and myristoyl LPA increased intracellular calcium by 1.5 to 2-fold in Edg7 expressing cells as compared to mock DNA-transfected cells (FIG. 9). Phosphatidic acid, too, behaved as an agonist at Edg7 receptors and increased intracellular calcium by approximately 5-fold (FIG. 10A). In contrast, sphingosine-1-phosphate, the endogenous agonist at Edg1, Edg3 and Edg5 receptors, failed to activate Edg7 receptors (FIG. 10B), indicating that Edg7 receptors respond to LPA in a selective manner.

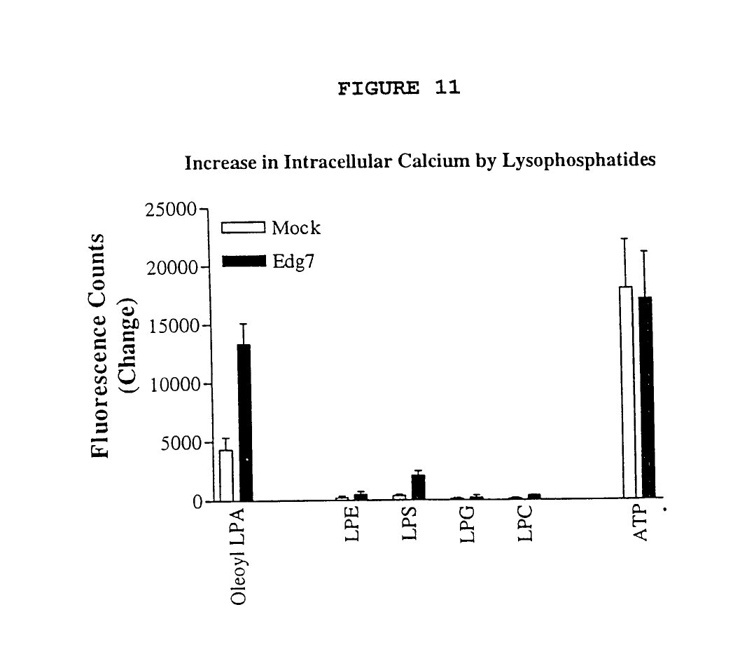

To further evaluate the specificity of the LPA response, Edg7 receptors were challenged with various lysophosphatides. None of the tested compounds, except for lysophosphatidylserine, produced a significantly different response in Edg7-expressing cells as compared to mock DNA-transfected cells (FIG. 11). This result is important for two reasons. First, it once again demonstrates the specificity of the response to LPA. Second, it argues against the possibility that LPA produced the response due to its detergent-like properties, since in that case, other lysophosphatides would have had the effect similar to LPA. In addition to these phospholipids, other lipid ligands, such as oleic acid, phosphatidyl choline, sphingomyelin, anandamide, platelet-activating factor, prostaglandin E₂ and thromboxane B₄ were tested against Edg7 and were found to be inactive (data not shown).

Activation of Calcium-Activated Cl⁻ Currents in Edg7-Expressing Xenopus oocytes

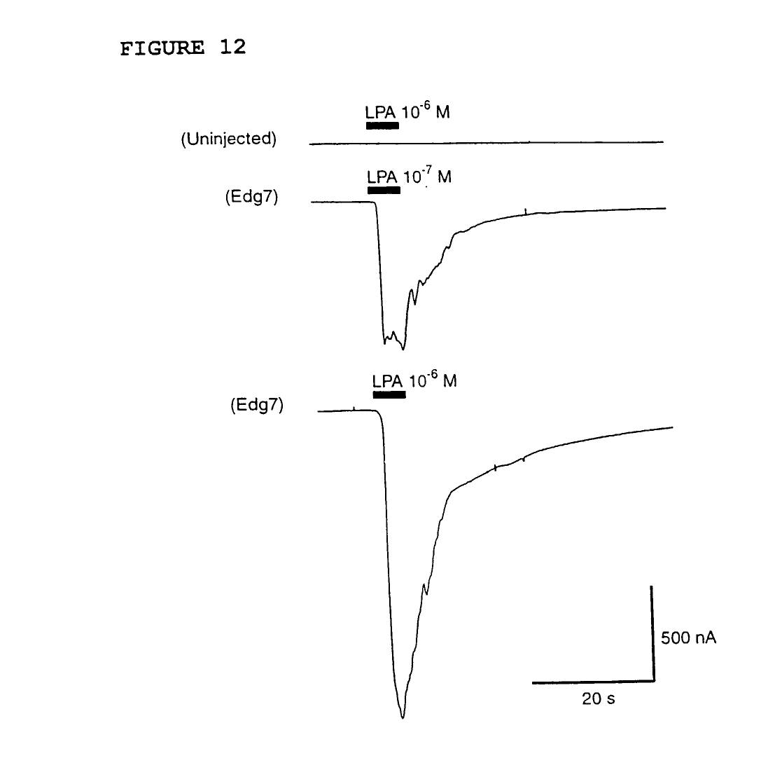

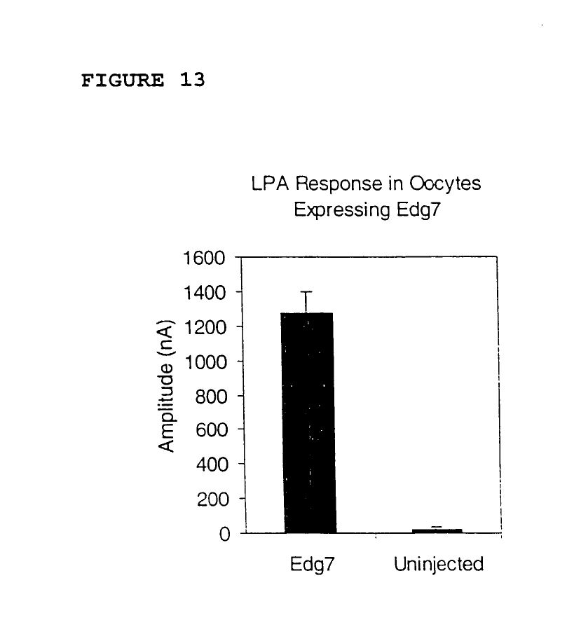

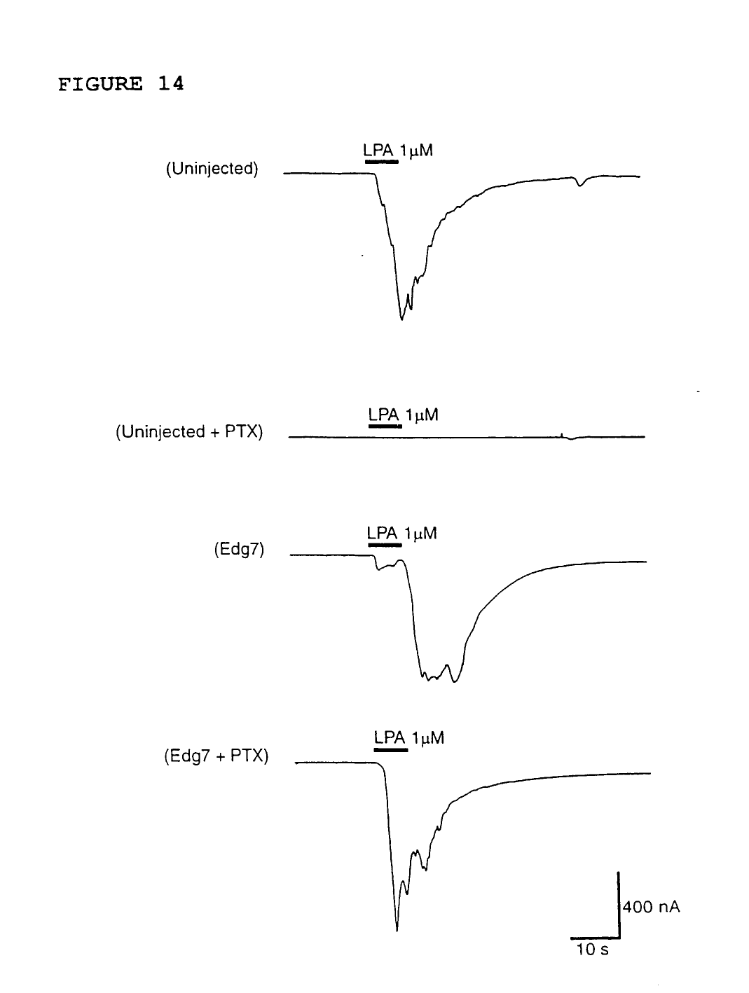

In Xenopus laevis oocytes, LPA activates at least two pharmacologically distinct receptor subtypes distinguished by 1-acyl-sn-glycero-2,3-cyclic phosphate (Liliom et al., 1996). Both of these ligands elicit oscillatory Cl⁻ currents in the oocyte through G protein-coupled stimulation of the phosphoinositide/Ca²⁺ second messenger system, which in turn leads to the activation of a Ca²⁺-dependent Cl⁻ current. As shown in FIG. 14, control oocytes, lacking injection of foreign mRNA, typically responded to LPA with inward Cl⁻ currents in the range of 100-1000 nA. This endogenous responsivity complicated the determination of LPA-induced activity specific to any particular cloned cDNA. In certain batches of ooctyes, however, endogenous LPA activity was quite low (FIG. 12), and in these cases the agonist sensitivity of Edg7 could be determined unambiguously. For the batch of ooctyes shown in FIG. 12, the Cl⁻ current amplitude in uninjected oocytes averaged 18±18 nA (n=5), whereas in oocytes injected with Edg7 mRNA, the current amplitude averaged 1272±126 nA (n=5; FIG. 13). A detailed concentration-response relation was not obtained, but nearly maximal responses were observed at an LPA concentration of 100 nM (FIG. 12). Other neurotransmitter and neurohormonal substances did not mimic the effect of LPA (data not shown).

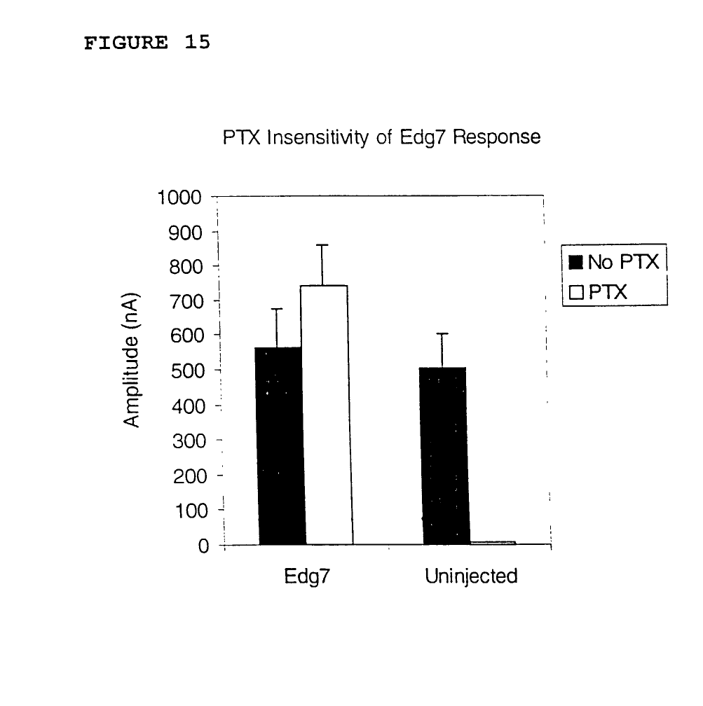

The intracellular Ca⁺⁺-releasing activity of Edg2 and Edg4 receptors (An et al., 1998b; Fukushima et al., 1998) is at least partially blocked by PTX. To investigate the PTX-sensitivity of the Edg7 receptor, batches of oocytes previously injected with Edg7 mRNA, as well as uninjected oocytes, were treated with PTX (25 ng injected 2-5 h prior to recording). In the experiment summarized in FIG. 14, uninjected (control) oocytes had a substantial response to LPA that masked the response specific to Edg7. After treatment with PTX, however, the LPA response in control oocytes was nearly abolished whereas in Edg7-injected oocytes a substantial current remained (FIGS. 14, 15). This result corroborates the previous report that LPA receptors endogenous to oocytes are sensitive to PTX (Durieux et al., 1992). Interestingly, although Edg7 is closest by homology to Edg2 and Edg4, it does not appear to share the feature of sensitivity to PTX. Taken together, the results in oocytes suggest that Edg7 couples to G_{q} or a related G-protein to stimulate the release of Ca⁺⁺ from intracellular stores, possibly via the activation of phospholipase C beta.

Detection of mRNA Coding for Human Edg7 Receptors

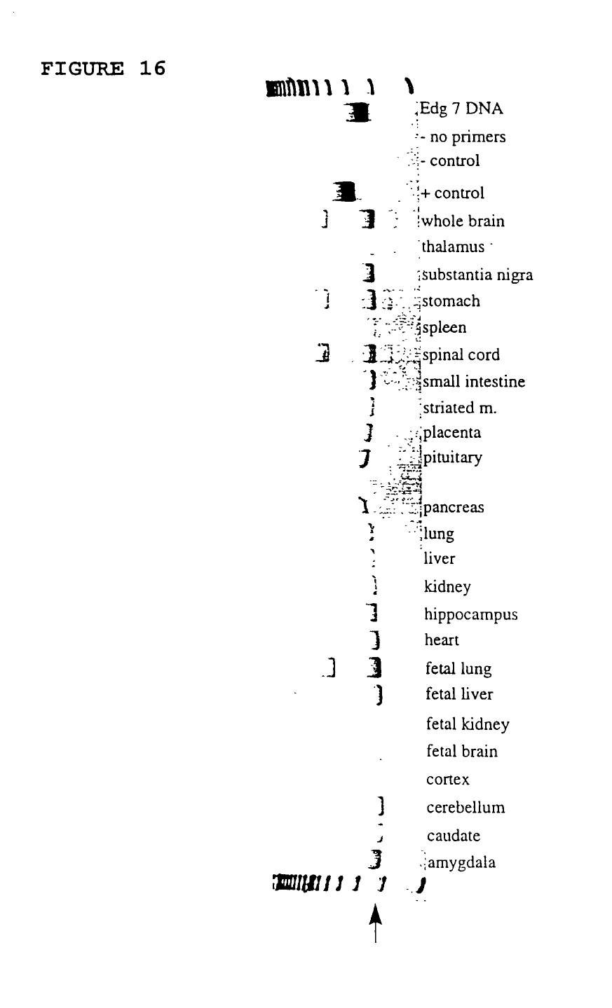

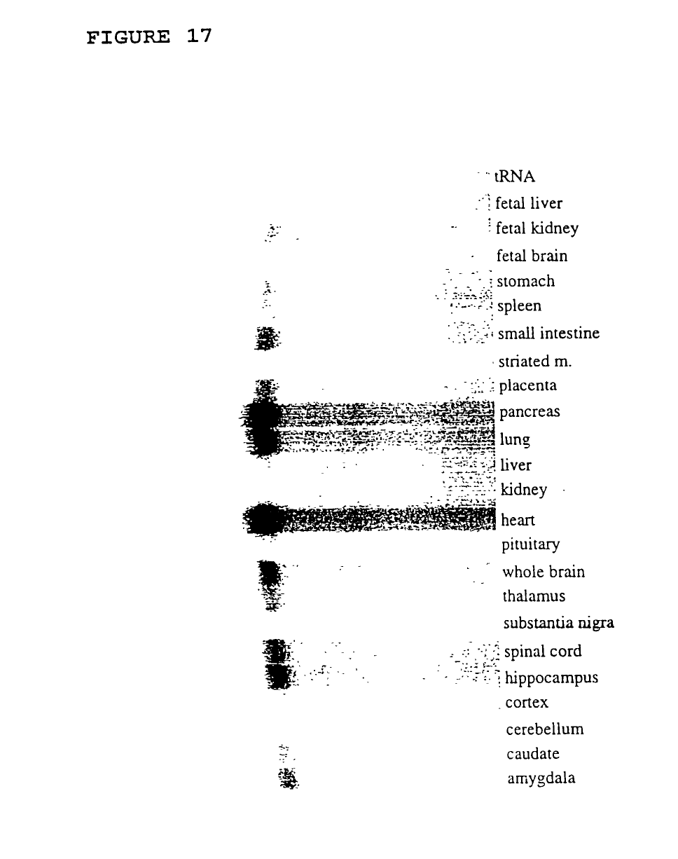

mRNA was isolated from multiple tissues (listed in table 1) and assayed as described. The distribution of mRNA encoding human Edg7 receptors is widespread. (Table 1, FIGS. 16, 17). Using RT-PCR, all tissues assayed revealed an amplicon consistent with Edg7 mRNA.

Using a solution hybridization/nuclease protection assay, Edg7 receptor mRNA was detected in most tissues assayed (Table 1). Highest levels of Edg7 mRNA are found in the heart, lungs, and pancreas, with lower levels detected elsewhere. The only regions not expressing Edg7 mRNA (as measured by RPA) are substantia nigra, pituitary, kidney, liver and striated muscle, although it was detected in these tissues using RT-PCR.

The distribution of Edg7 mRNA suggests a broad regulatory function in multiple organ systems in the body. It is found in most tissues assayed. The presence of high levels of Edg7 mRNA in the heart and lung implicate a cardiovascular regulatory function. It is interesting to note that Edg7 mRNA is localized to cardiac muscle and not striated muscle. The difference between the two muscle types is striking with no detectable Edg7 mRNA found in striated muscle, with extremely high levels found in the heart. The localization of Edg7 to smooth muscle was not determined. Although it is found in moderate amounts in stomach and intestinal tissue, it is not known if it is localized to smooth muscle or mucosal/submucosal layers. Another area expressing very high levels of Edg7 mRNA is the pancreas. The pancreas secretes a broad variety of broadly active substances, indicating that Edg7 receptor may play a role in regulating multiple metabolic functions, potentially via endocrine mechanisms. The presence of Edg7 mRNA in multiple of regions of the CNS including the spinal cord, hippocampal formation (where levels are highest in the CNS) and other functionally diverse areas, indicate a diffuse regulatory function or regional functionality for this receptor.

Edg7 mRNA appears to be developmentally regulated. In fetal brain and lung, Edg7 mRNA is detectable at low/very low levels using RPA. This is in stark contrast to the high levels detected in adult brain and lung. This pattern is reversed in kidney and liver. There is a low to moderate amount of mRNA coding for Edg7 in fetal tissue with no detectable mRNA in adult liver and kidney. The time course of this increase has not been examined and would be important in understanding the function of this receptor.

In summary, the broad distribution of Edg7 receptor mRNA implies broad regulatory functions that involves multiple organ system, endocrine mechanisms as well as the central nervous system.

TABLE 1
Distribution of mRNA coding for human Edg7
receptors using Rt-PCR and RPA.

| Region | PCR | RPA | Potential applications |
|---|---|---|---|
| liver | ++ | − | Diabetes |
| kidney | ++ | − | Hypertension, electrolyte |
| | | | balance |
| lung | +++ | +++ | Respiratory disorders, asthma |
| heart | +++ | ++++ | Cardiovascular indications |
| pancreas | ++ | +++ | Diabetes, endocrine disorders |
| placenta | ++ | ++ | Gestational abnormalities |
| small | +++ | ++ | Gastrointestinal disorders |
| intestine | | | |
| spleen | + | + | Immune function |
| stomach | +++ | ++ | Gastrointestinal disorders |
| striated | ++ | − | Musculoskeletal disorders |
| muscle | | | |
| pituitary | +++ | − | Endocrine/neuroendocrine |
| | | | regulation |
| whole brain | +++ | +++ | |
| amygdala | ++++ | ++ | Depression, phobias, anxiety, |
| | | | mood disorders |
| cerebral | ++ | + | Sensory and motor integration, |
| cortex | | | cognition |
| hippocampus | +++ | +++ | Cognition/memory |
| hypothalamus | +++ | na | Appetite/obesity, neuroendocrine |
| | | | regulation |
| spinal cord | +++ | ++ | Analgesia, sensory modulation |
| | | | and transmission |
| cerebellum | +++ | + | Motor coordination |
| thalamus | + | ++ | Sensory integration |
| substantia | +++ | +/− | Modulation of dopaminergic |
| nigra | | | function, modulation of motor |
| | | | coordiantion. |
| caudate- | ++ | ++ | Modulation of dopaminergic |
| putamen | | | function |
| fetal brain | + | +/− | Developmental disorders |
| fetal lung | ++++ | + | Developmental disorders |
| fetal kidney | + | + | Developmental disorders |
| fetal liver | ++ | + | Developmental disorders |

### REFERENCES

Alexander, J. S., et al., “Platelet-derived lysophosphatidic acid decreases endothelial permeability in vitro” Am. J. Physiol. 274:H115-122 (1998).

Amano, M., et al., “Formation of actin stress fibers and focal adhesions enhanced by Rho-kinase” Science 275:1308-1311 (1997).

An, S., et al., “Molecular cloning of the human Edg2 protein and its identification as a functional cellular receptor for lysophosphatidic acid” Biochem. Biophys. Res. Commun. 231:619-622 (1997a).

An, S., et al., “Identification of cDNAs encoding two G protein-coupled receptors for lysosphingolipids” FEBS Lett. 417:279-282 (1997b).

An, S., et al., “Characterization of a novel subtype of human G protein-coupled receptor for lysophosphatidic acid” J. Biol. Chem. 273:7906-7910 (1998a).

An, S., et al., “Recombinant human G protein-coupled lysophosphatidic acid receptors mediate intracellular calcium mobilization” Mol. Pharmacol. 54:881-888 (1998b).

Bradford, M. M., “A rapid and sensitive method for the quantitation of microgram quantities of protein utilizing the principle of protein-dye binding”, Anal. Biochem. 72: 248-254 (1976).

Burns, C. C. et al., “Indentification and deletion of sequences required for feline leukemia virus RNA packaging and construction of a high-titer feline leukemia virus packaging cell line” Virology 222(1):14-20 (1996).

Bush, et al., “Nerve growth factor potentiates bradykinin-induced calcium influx and release in PC12 cells” J. Neurochem. 57: 562-574(1991).

Cerutis, D. R., et al., “Lysophosphatidic acid and EGF stimulate mitogenesis in human airway smooth muscle” Am. J. Physiol. 273:L10-L15 (1997).

Chu, Y. Y. et al., “Characterization of the rat A2a adenosine receptor gene” DNA Cell Biol. 15(4):329-37 (1996).

Chuprun, J. K., et al., “The heterotrimeric G protein Gₐₗₚₕₐ₁₂ mediates lysophosphatidic acid-stimulated induction of the c-fos gene in mouse fibroblasts” J. Biol. Chem. 272:773-781 (1997).

Dascal, N., et al., “Atrial G protein-activated K⁺ channel: expression cloning and molecular properties” Proc. Natl. Acad. Sci. USA 90:10235-10239 (1993).

Durieux, M. E., et al., “Lysophosphatidic acid induces a pertussis toxin-sensitive Ca²⁺-activated Cl⁻ current in Xenopus leavis oocytes” Am. J. Physiol. 263:C896-C900 (1992).

Erickson, J. R., et al., “Edg-2/Vzg-1 couples to the yeast pheromone response pathway selectively in response to lysophosphatidic acid” J. Biol. Chem. 273:1506-1510 (1998).

Fong, T. M. et al., “Mutational analysis of neurokinin receptor function” Can. J. Physio. Pharmacol. 73(7):860-865 (1995).

Fourcade, O., et al., “Secretory phospholipase A₂ generates the novel lipid mediator lysophosphatidic acid in membrane microvesicles shed from activated cells” Cell 80:919-927 (1995).

Fromm, C., et al., “The small GTP-binding protein rho links G protein-coupled receptors and Gₐₗₚₕₐ₁₂ to the serum response element and to cellular transformation” Proc. Natl. Acad. Sci. USA 94:10098-10103 (1997).

Fukushima, N., et al., “A single receptor encoded by vzg-1/lp_{A1}/edg-2 couples to G-proteins and mediates multiple cellular responses to lysophosphatidic acid” Proc. Natl. Acad. Sci. USA 95:6151-6156 (1998).

Gaits, F., et al., “Lysophosphatidic acid as a phospholipid mediator: Pathways of synthesis” FEBS Lett. 410:54-58 (1997a).

Gaits, F., et al., “Dual effect of lysophosphatidic acid on proliferation of glomerular mesangial cells” Kidney Int. 51:1022-1027 (1997b).

Gerrard, J. M., et al., “Lysophosphatidic acids. Influence on platalet aggregation and intracellular calcium flux” Am. J. Pathol. 96:423-438 (1979).

Goetzl, E. J. and An, S., “Diversity of cellular receptors and functions for the lysophospholipid growth factors lysophosphatidic acid and sphingosine 1-phosphate” FASEB J. 12:1589-1598 (1998).

Gohla, A., et al., “The G-protein G₁₃ but not G₁₂ mediates signaling from lysophosphatidic acid receptor via epidermal growth factor receptor to rho” J. Biol. Chem. 273:4653-4659 (1998).

Graler, M. H., et al., “EDG6, a novel G-protein-coupled receptor related to receptors for bioactive lysophospholipids, is specifically expressed in lymphoid tissue” Genomics 53:164-169 (1998).

Graziano, M. P. et al., “The amino terminal domain of the glucagon-like peptide-1 receptor is a critical determinant of subtype specificity” Receptors Channels 4(1):9-17 (1996).

Guan X. M. et al., “Determination of Structural Domains for G Protein Coupling and Ligand Binding in β₃-Adrenergic Receptor” Mol. Pharmacol. 48(3) :492-498 (1995).

Gundersen, C. B., et al., “Serotonin receptors induced by exogenous messenger RNA in Xenopus oocytes” Proc. R. Soc. Lond. B. Biol. Sci. 219(1214): 103-109 (1983).

Guo, Z., et al., “Molecular cloning of a high-affinity receptor for the growth factor-like lipid mediator lysophosphatidic acid from Xenopus oocytes” Proc. Natl. Acad. Sci. USA 93:14367-14372 (1996).

Hecht, J. H., et al., “Ventricular zone gene-1 (vzg-1) encodes a lysophosphatidic acid receptors expressed in neurogenic regions of the developing cerebral cortex” J. Cell Biol. 135:1071-1083 (1996).

Holtsberg, F. W., et al., “Lysophosphatidic acid induces a sustained elevation of neuronal intracellular calcium” J. Neurochem. 69:68-75 (1997).

Holtsberg, F. W., et al., “Lysophosphatidic acid induces necrosis and apoptosis in hippocampal neurons” J. Neurochem. 70:66-76 (1998).

Hordijk, P. L., et al., “Protein tyrosine phosphorylation induced by lysophosphatidic acid in Rat-1 fibroblasts: Evidence that phosphorylation of MAP kinase is mediated by the G₁-p21ras pathway” J. Biol. Chem. 269:645-651 (1994).

Howe, L. R. and Marshall, C. J., “Lysophosphatidic acid stimulates mitogen-activated protein kinase activation via a G-protein-coupled pathway requiring p21ras and p74raf-1” J. Biol. Chem. 268:20717-20720 (1993).

Inanobe, A., et al., “Characterization of G-protein-gated K⁺ channels composed of Kir3.2 subunits in dopaminergic neurons of the substantia nigra” J. of Neuroscience 19(3):1006-1017 (1999).

Inoue, C. N., et al., “Effects of lysophosphatidic acid, a novel lipid mediator, on cytosolic Ca²⁺ and contractility in cultured rat mesangial cells” Circ. Res. 77:888-896 (1995).

Jalink, K., et al., “Growth factor-like effects of lysophosphatidic acid, a novel lipid mediator” Biochim. Biophys. Acta 1198:185-196 (1994).

Jalink, K., et al., “Lysophosphatidic acid-induced Ca²⁺ mobilization in human A431 cells: structure-activity analysis” Biochem. J. 307:609-616 (1995).

Kawasawa, Y., et al., “Cloning and characterization of a mouse homologue of Xenopus PSP24 LPA receptor” FASEB J. 12:A1460 (1998).

Keller, J. N., et al., “Lysophosphatidic acid-induced proliferation-related signals in astrocytes” J. Neurochem. 69:1073-1084 (1997).

Koh, J. S., et al., “Lysophosphatidic acid is a major serum noncytokine survival factor for murine macrophages which acts via the phosphatidylinositol 3-kinase signaling pathway” J. Clin. Invest. 102:716-727 (1998).

Krapivinsky, G., et al., “The G-protein-gated atrial K⁺ channel IKACh is a heteromultimer of two inwardly rectifying K(+)-channel proteins” Nature 374:135-141 (1995).

Krapivinsky, G., et al., “The cardiac inward rectifier K⁺ channel subunit, CIR, does not comprise the ATP-sensitive K⁺ channel, IKATP” J. Biol. Chem. 270:28777-28779 (1995b).

Kubo, Y., et al., “Primary structure and functional expression of a rat G-protein-coupled muscarinic potassium channel” Nature 364:802-806 (1993).

Lazareno, S. and Birdsall, N. J. M. “Pharmacological characterization of acetylcholine stimulated [³⁵S]-GTPγS binding mediated by human muscarinic m1-m4 receptors: antagonist studies”, Br. J. Pharmacol. 109: 1120-1127 (1993).

Lee, M. -J., et al., “Sphingosine-1-phosphate as a ligand for the G protein-coupled receptor EDG-1” Science 279:1552-1555 (1998).

Levine, J. S., et al., “Lysophosphatidic acid: a novel growth and survival factor for renal proximal tubular cells” Am. J. Physiol. 273:F575-F585 (1997).

Liliom, K., et al., “Xenopus oocytes express multiple receptors for LPA-like lipid mediators” Am. J. Physiol. 270:C772-C777 (1996).

Liliom, K., et al., “Growth factor-like phospholipids generated after corneal injury” Am. J. Physiol. 274:C1065-C1074 (1998).

Mao, J., et al., “Specific involvement of G proteins in regulation of serum response factor-mediated gene transcription by different receptors” J. Biol. Chem. 273:27118-27123 (1998).

Milligan, G., et al., “Use of chimeric Gα proteins in drug discovery” TIPS (In press).

Moolenaar, W. H., “Lysophosphatidic acid, a multifunctional phospholipid messenger” J. Biol. Chem. 270:12949-12952 (1995).

Moolenaar, W. H., et al., “Lysophosphatidic acid: G-protein signalling and cellular responses” Curr. Opin. Cell Biol. 9:168-173 (1997).

Narumiya, S., et al., “Rho effectors and reorganization of actin cytoskeleton” FEBS Lett. 410:68-72 (1997).

Nietgen, G. W. and Durieux, M. E., “Intercellular signaling by lysophosphatidate. Recent developments” Cell Adhesion Commun. 5:221-235 (1998).

Nishizaki, T. and Sumikawa, K., “Lysophosphatidic acid potentiates Ach receptor currents by G-protein-mediated activation of protein kinase C” Brain Res. Mol. Brain Res. 50:121-126 (1997).

Perkins, L. M., et al., “Activation of serum response element-regulated genes by lysophosphatidic acid” Nucleic Acids Res. 22(3): 450-452 (1994).

Piazza, G. A., et al., “Lysophosphatidic acid induction of transforming growth factors alpha and beta: modulation of proliferation and differentiation in cultured human keratinocytes and mouse skin” Exp. Cell Res. 216:51-64 (1995).

Pietruck, F., et al., “Signalling properties of lysophosphatidic acid in primary human skin fibroblasts: role of pertussis toxin-sensitive GTP-binding proteins” Naunyn-Schmiedeberg's Arch. Pharmacol. 355:1-7 (1997).

Plevin, R., et al., “Differences in the regulation of endothelin-1- and lysophosphatidic acid-stimulated Ins(1,4,5)P₃ formation in Rat-1 fibroblasts” Biochem. J. 280:609-615 (1991).

Postma, F. R., et al., “Serum-induced membrane depolarization in quiescent fibroblasts: activation of a chloride conductance through the G protein-coupled LPA receptor” EMBO J. 15:63-72 (1996).

Quick, M. W. and Lester, H. A., “Methods for expression of excitability proteins in Xenopus oocytes”, Meth. Neurosci. 19: 261-279 (1994).

Ridley, A. J. and Hall, A., “The small GTP-binding protein rho regulates the assembly of focal adhesions and actin stress fibers in response to growth factors” Cell 70:389-399 (1992).

Sakai, T., et al., “Restoration of beta₁A integrins is required for lysophosphatidic acid-induced migration of beta₁-null mouse fibroblastic cells” J. Biol. Chem. 273:19378-19382 (1998).

Salon, J. A. and Owicki, J. A., “Real-time measurements of receptor activity: Application of microphysiometic techniques to receptor biology” Meth. Neurosci. 25: pp. 201-224, Academic Press (1996).

Schulze, C., et al., “Lysophosphatidic acid increases tight junction permeability in cultured brain endothelial cells” J. Neurochem. 68:991-1000 (1997).

Schumacher, K. A., et al., “Platelet aggregation evoked in vitro and in vivo by phosphatidic acids and lysoderivatives: identity with substances in aged serum (DAS)” Thromb. Haemost. 42:631-640 (1979).

Shiono, S., et al., “Neurotransmitter release from lysophosphatidic acid stimulated PC12 cells: involvement of lysophosphatidic acid receptors” Biochem. Biophys. Res. Commun. 193:667-673 (1993).

Simon, M. F., et al., “Platelet aggregating activity of lysophosphatidic acids is not related to their calcium ionophore properties” FEBS Lett. 166:115-119 (1984).

Smith, K. E., et al., “Expression cloning of a rat hypothalamic galanin receptor coupled to phosphoinositide turnover”, J. Biol. Chem. 272: 24612-24616 (1997).

Spurney, R. F. et al., “The C-terminus of the thromboxane receptor contributes to coupling and desensitization in a mouse mesangial cell line” J. Pharmacol. Exp. Ther. 283(1):207-215 (1997).

Stam, J. C., et al., “Invasion of T-lymphoma cells: cooperation between Rho family GTPases and lysophospholipid receptor signaling” EMBO J. 17:4066-4074 (1998).

Takahashi, T., et al., “Rat brain serotonin receptors in Xenopus oocytes are coupled by intracellular calcium to endogenous channels” Proc. Natl. Acad. Sci. USA 84(14): 5063-5067 (1987).

Thomson, F. J., et al., “Identification and characterization of a lysophosphatidic acid receptor” Mol. Pharmacol. 45:718-723 (1994).

Thoreson, W. B., et al., “Lysophosphatidic acid stimulates proliferation of human retinal pigment epithelial cells” Curr. Eye Res. 16:698-702 (1997).

Tian, W., et al., “Determinants of alpha-Adrenergic Receptor Activation of G protein: Evidence for a Precoupled Receptor/G protein State” Molecular Pharmacology 45: 524-553 (1994).

Tigyi, G., et al., “Lysophosphatidic acid possesses dual action in cell proliferation” Proc. Natl. Acad. Sci. USA 91:1908-1912 (1994).

Tigyi, G., et al., “Lysophosphatidic acid alters cerebrovascular reactivity in piglets” Am. J. Physiol. 268:H2048-H2055 (1995).

Toews, M. L., et al., “Lysophosphatidic acid enhances contractility of isolated airway smooth muscle” J. Appl. Physiol. 83:1216-1222 (1997).

Tokumura, A., et al., “Effects of synthetic and natural lysophosphatidic acids on the arterial blood pressure of different animal species” Lipids 13:572-574 (1978).

Tokumura, A., et al., “Vasopressor effect of lysophosphatidic acid on spontaneously hypertensive rats and Wistar Kyoto rats” Res. Commun. Mol. Pathol. Pharmacol. 90:96-102 (1995).

Underwood, D. J. et al., “Structural model of antagonist and agonist binding to the angiotensin II, AT1 subtype, G protein coupled receptor” Chem. Biol. 1(4)1:211-21 (1994).

Valet, P., et al., “Alpha₂-adrenergic receptor-mediated release of lysophosphatidic acid by adipocytes. A paracrine signal for preadipocyte growth” J. Clin. Invest. 101:1431-1438 (1998).

van Corven, E. J., et al., “Mitogenic action of lysophosphatidic acid and phosphatidic acid on fibroblasts. Dependence on acyl-chain length and inhibition by suramin” Biochem. J. 281:163-169 (1992).

Van Corven, E. J., et al., “Pertussis toxin-sensitive activation of p21ras by G protein-coupled receptor agonists in fibroblasts” Proc. Natl. Acad. Sci. USA 90:1257-1261 (1993).

van der Bend, R. L., et al., “Identification of a putative membrane receptor for the bioactive phospholipid, lysophosphatidic acid” EMBO J. 11:2495-2501 (1992).

Westermann, A. M., et al., “Malignant effusions contain lysophosphatidic acid (LPA)-like activity” Ann. Oncol. 9:437-442 (1998).

Xu, Y., et al., “Lysophosphatidic acid as a potential biomarker for ovarian and other gynecologic cancers” JAMA 280:719-723 (1998).

Yakubu, M. A., et al., “Role of lysophosphatidic acid in endothelin-1- and hematoma-induced alteration of cerebral microcirculation” Am. J. Physiol. 273:R703-R709 (1997).

Yoshioka, K., et al., “Small GTP-binding protein rho stimulates the actomyosin system, leading to invasion of tumor cells” J. Biol. Chem. 273:5146-5154 (1998).

Zhou, D., et al., “Phosphatidic acid and lysophosphatidic acid induce haptotactic migration of human monocytes” J. Biol. Chem. 270:25549-25556 (1995).

What is claimed is:

1. An isolated nucleic acid encoding a mammalian Edg7 receptor.

2. The nucleic acid of claim 1, wherein the nucleic acid is DNA.

3. The DNA of claim 2, wherein the DNA is cDNA.

4. The DNA of claim 2, wherein the DNA is genomic DNA.

5. The nucleic acid of claim 1, wherein the nucleic acid is RNA.

6. The nucleic acid of claim 1, wherein the mammalian Edg7 receptor is a human Edg7 receptor.

7. The nucleic acid of claim 6, wherein the human Edg7 receptor has an amino acid sequence identical to that encoded by the plasmid hSNORF3-pCDNA3.1 (ATCC Accession No. 203520).

8. The nucleic acid of claim 6, wherein the human Edg7 receptor has an amino acid sequence identical to the amino acid sequence shown in FIG. 2 (SEQ ID NO: 2).

9. A purified mammalian Edg7 receptor protein.

10. The purified mammalian Edg7 receptor protein of claim 9, wherein the Edg7 receptor protein is a human Edg7 receptor protein.

11. A vector comprising the nucleic acid of claim 1.

12. A vector comprising the nucleic acid of claim 6.

13. A vector of claim 11 or 12 adapted for expression in a cell which comprises the regulatory elements necessary for expression of the nucleic acid in the cell operatively linked to the nucleic acid encoding the receptor so as to permit expression thereof, wherein the cell is a bacterial, amphibian, yeast, insect or mammalian cell.

14. The vector of claim 13, wherein the vector is a baculovirus.

15. The vector of claim 11, wherein the vector is a plasmid.

16. The plasmid of claim 15 designated hSNORF3-pCDNA3.1 (ATCC Accession No. 203520)

17. A cell comprising the vector of claim 13.

18. A cell of claim 17, wherein the cell is a non-mammalian cell.

19. A cell of claim 18, wherein the non-mammalian cell is a Xenopus oocyte cell or a Xenopus melanophore cell.

20. A cell of claim 17, wherein the cell is a mammalian cell.

21. A mammalian cell of claim 20, wherein the cell is a COS-7 cell, a 293 human embryonic kidney cell, a NIH-3T3 cell, a LM(tk-) cell, a mouse Y1 cell, or a CHO cell.

22. A cell of claim 17, wherein the cell is an insect cell.

23. An insect cell of claim 22, wherein the insect cell is an Sf9 cell, an Sf21 cell or a Trichoplusia ni 5B-4 cell.

24. A membrane preparation isolated from the cell of any of claims 17, 18, 20, 21, 22 or 23.

25. A nucleic acid probe comprising at least 15 nucleotides, which probe specifically hybridizes with a nucleic acid encoding a mammalian Edg7 receptor, wherein the probe has a unique sequence corresponding to a sequence present within one of the two strands of the nucleic acid encoding the mammalian Edg7 receptor and contained in plasmid hSNORF3-pCDNA3.1 (ATCC Accession No. 203520).

26. A nucleic acid probe comprising at least 15 nucleotides, which probe specifically hybridizes with a nucleic acid encoding a mammalian Edg7 receptor, wherein the probe has a unique sequence corresponding to a sequence present within (a) the nucleic acid sequence shown in FIG. 1 (SEQ ID NO: 1) or (b) the reverse complement thereto.

27. The nucleic acid probe of claim 26, wherein the nucleic acid is DNA.

28. The nucleic acid probe of claim 26, wherein the nucleic acid is RNA.

29. An antisense oligonucleotide having a sequence capable of specifically hybridizing to the RNA of claim 5, so as to prevent translation of the RNA.

30. An antisense oligonucleotide having a sequence capable of specifically hybridizing to the genomic DNA of claim 4, so as to prevent transcription of the genomic DNA.

31. An antisense oligonucleotide of claim 29 or 30, wherein the oligonucleotide comprises chemically modified nucleotides or nucleotide analogues.

32. An antibody capable of binding to a mammalian Edg7 receptor encoded by the nucleic acid of claim 1.

33. An antibody of claim 32, wherein the mammalian Edg7 receptor is a human Edg7 receptor.

34. An agent capable of competitively inhibiting the binding of the antibody of claim 32 to a mammalian Edg7 receptor.

35. An antibody of claim 32, wherein the antibody is a monoclonal antibody or antisera.

36. A pharmaceutical composition comprising (a) an amount of the oligonucleotide of claim 29 capable of passing through a cell membrane and effective to reduce expression of a mammalian Edg7 receptor and (b) a pharmaceutically acceptable carrier capable of passing through the cell membrane.

37. A pharmaceutical composition of claim 36, wherein the oligonucleotide is coupled to a substance which inactivates mRNA.

38. A pharmaceutical composition of claim 37, wherein the substance which inactivates mRNA is a ribozyme.

39. A pharmaceutical composition of claim 37, wherein the pharmaceutically acceptable carrier comprises a structure which binds to a mammalian Edg7 receptor on a cell capable of being taken up by the cells after binding to the structure.

40. A pharmaceutical composition of claim 39, wherein the pharmaceutically acceptable carrier is capable of binding to a mammalian Edg7 receptor which is specific for a selected cell type.

41. A pharmaceutical composition which comprises an amount of the antibody of claim 32 effective to block binding of a ligand to a human Edg7 receptor and a pharmaceutically acceptable carrier.

42. A transgenic, nonhuman mammal expressing DNA encoding a mammalian Edg7 receptor of claim 1.

43. A transgenic, nonhuman mammal comprising a homologous recombination knockout of the native mammalian Edg7 receptor.

44. A transgenic, nonhuman mammal whose genome comprises antisense DNA complementary to the DNA encoding a mammalian Edg7 receptor of claim 1 so placed within the genome as to be transcribed into antisense mRNA which is complementary to mRNA encoding the mammalian Edg7 receptor and which hybridizes to mRNA encoding the mammalian Edg7 receptor, thereby reducing its translation.

45. The transgenic, nonhuman mammal of claim 42 or 43, wherein the DNA encoding the mammalian Edg7 receptor additionally comprises an inducible promoter.

46. The transgenic, nonhuman mammal of claim 42 or 43, wherein the DNA encoding the mammalian Edg7 receptor additionally comprises tissue specific regulatory elements.

47. A transgenic, nonhuman mammal of claim 42, 43, or 44, wherein the transgenic, nonhuman mammal is a mouse.

48. A process for identifying a chemical compound which specifically binds to a mammalian Edg7 receptor which comprises contacting cells containing DNA encoding and expressing on their cell surface the mammalian Edg7 receptor, wherein such cells do not normally express the mammalian Edg7 receptor, with the compound under conditions suitable for binding, and detecting specific binding of the chemical compound to the mammalian Edg7 receptor.

49. A process for identifying a chemical compound which specifically binds to a mammalian Edg7 receptor which comprises contacting a membrane preparation from cells containing DNA encoding and expressing on their cell surface the mammalian Edg7 receptor, wherein such cells do not normally express the mammalian Edg7 receptor, with the compound under conditions suitable for binding, and detecting specific binding of the chemical compound to the mammalian Edg7 receptor.

50. The process of claim 48 or 49, wherein the mammalian Edg7 receptor is a human Edg7 receptor.

51. The process of claim 48 or 49, wherein the mammalian Edg7 receptor has substantially the same amino acid sequence as the human Edg7 receptor encoded by plasmid hSNORF3-pCDNA3.1 (ATCC Accession No. 203520).

52. The process of claim 48 or 49, wherein the mammalian Edg7 receptor has substantially the same amino acid sequence as that shown in FIG. 2 (SEQ ID NO: 2).

53. The process of claim 48 or 49, wherein the mammalian Edg7 receptor has the amino acid sequence shown in FIG. 2 (SEQ ID NO: 2).

54. The process of claim 48 or 49, wherein the compound is not previously known to bind to a mammalian Edg7 receptor.

55. A compound identified by the process of claim 54.

56. A process of claim 48 or 49, wherein the cell is an insect cell.

57. The process of claim 48 or 49, wherein the cell is a mammalian cell.

58. The process of claim 57, wherein the cell is nonneuronal in origin.

59. The process of claim 58, wherein the nonneuronal cell is a COS-7 cell, 293 human embryonic kidney cell, a CHO cell, a NIH-3T3 cell, a mouse Y1 cell, or a LM(tk-) cell.

60. A process of claim 57, wherein the compound is a compound not previously known to bind to a mammalian Edg7 receptor.

61. A compound identified by the process of claim 60.

62. A process involving competitive binding for identifying a chemical compound which specifically binds to a mammalian Edg7 receptor which comprises separately contacting cells expressing on-their cell surface the mammalian Edg7 receptor, wherein such cells do not normally express the mammalian Edg7 receptor, with both the chemical compound and a second chemical compound known to bind to the receptor, and with only the second chemical compound, under conditions suitable for binding of both compounds, and detecting specific binding of the chemical compound to the mammalian Edg7 receptor, a decrease in the binding of the second chemical compound to the mammalian Edg7 receptor in the presence of the chemical compound indicating that the chemical compound binds to the mammalian Edg7 receptor.

63. A process involving competitive binding for identifying a chemical compound which specifically binds to a mammalian Edg7 receptor which comprises separately contacting a membrane preparation from cells expressing on their cell surface the mammalian Edg7 receptor, wherein such cells do not normally express the mammalian Edg7 receptor, with both the chemical compound and a second chemical compound known to bind to the receptor, and with only the second chemical compound, under conditions suitable for binding of both compounds, and detecting specific binding of the chemical compound to the mammalian Edg7 receptor, a decrease in the binding of the second chemical compound to the mammalian Edg7 receptor in the presence of the chemical compound indicating that the chemical compound binds to the mammalian Edg7 receptor.

64. A process of claim 62 or 63, wherein the mammalian Edg7 receptor is a human Edg7 receptor.

65. The process of claim 62 or 63, wherein the cell is an insect cell.

66. The process of claim 62 or 63, wherein the cell is a mammalian cell.

67. The process of claim 66, wherein the cell is nonneuronal in origin.

68. The process of claim 67, wherein the nonneuronal cell is a COS-7 cell, 293 human embryonic kidney cell, a CHO cell, a NIH-3T3 cell, a mouse Y1 cell, or a LM(tk-) cell.

69. The process of claim 68, wherein the compound is not previously known to bind to a mammalian Edg7 receptor.

70. A compound identified by the process of claim 69.

71. A method of screening a plurality of chemical compounds not known to bind to a mammalian Edg7 receptor to identify a compound which specifically binds to the mammalian Edg7 receptor, which comprises
(a) contacting cells transfected with and expressing DNA encoding the mammalian Edg7 receptor with a compound known to bind specifically to the mammalian Edg7 receptor;
(b) contacting the preparation of step (a) with the plurality of compounds not known to bind specifically to the mammalian Edg7 receptor, under conditions permitting binding of compounds known to bind to the mammalian Edg7 receptor;
(c) determining whether the binding of the compound known to bind to the mammalian Edg7 receptor is reduced in the presence of any compound within the plurality of compounds, relative to the binding of the compound in the absence of the plurality of compounds; and if so
(d) separately determining the binding to the mammalian Edg7 receptor of compounds included in the plurality of compounds, so as to thereby identify the compound which specifically binds to the mammalian Edg7 receptor.

72. A method of screening a plurality of chemical compounds not known to bind to a mammalian Edg7 receptor to identify a compound which specifically binds to the mammalian Edg7 receptor, which comprises
(a) contacting a membrane preparation from cells transfected with and expressing DNA encoding the mammalian Edg7 receptor with the plurality of compounds not known to bind specifically to the mammalian Edg7 receptor under conditions permitting binding of compounds known to bind to the mammalian Edg7 receptor;
(b) determining whether the binding of a compound known to bind to the mammalian Edg7 receptor is reduced in the presence of any compound within the plurality of compounds, relative to the binding of the compound in the absence of the plurality of compounds; and if so
(c) separately determining the binding to the mammalian Edg7 receptor of compounds included in the plurality of compounds, so as to thereby identify the compound which specifically binds to the mammalian Edg7 receptor.

73. A method of claim 71 or 72, wherein the mammalian Edg7 receptor is a human Edg7 receptor.

74. A method of claim 71 or 72, wherein the cell is a mammalian cell.

75. A method of claim 74, wherein the mammalian cell is non-neuronal in origin.

76. The method of claim 75, wherein the non-neuronal cell is a COS-7 cell, a 293 human embryonic kidney cell, a LM(tk-) cell, a CHO cell, a mouse Y1 cell, or an NIH-3T3 cell.

77. A method of detecting expression of a mammalian Edg7 receptor by detecting the presence of mRNA coding for the mammalian Edg7 receptor which comprises obtaining total mRNA from the cell and contacting the mRNA so obtained with the nucleic acid probe of claim 25 or 26 under hybridizing conditions, detecting the presence of mRNA hybridizing to the probe, and thereby detecting the expression of the mammalian Edg7 receptor by the cell.

78. A method of detecting the presence of a mammalian Edg7 receptor on the surface of a cell which comprises contacting the cell with the antibody of claim 32 under conditions permitting binding of the antibody to the receptor, detecting the presence of the antibody bound to the cell, and thereby detecting the presence of the mammalian Edg7 receptor on the surface of the cell.

79. A method of determining the physiological effects of varying levels of activity of mammalian Edg7 receptors which comprises producing a transgenic, nonhuman mammal of claim 45 whose levels of mammalian Edg7 receptor activity are varied by use of an inducible promoter which regulates mammalian Edg7 receptor expression.

80. A method of determining the physiological effects of varying levels of activity of mammalian Edg7 receptors which comprises producing a panel of transgenic, nonhuman mammals of claim 45 each expressing a different amount of mammalian Edg7 receptor.

81. A method for identifying an antagonist capable of alleviating an abnormality wherein the abnormality is alleviated by decreasing the activity of a mammalian Edg7 receptor comprising administering a compound to the transgenic, nonhuman mammal of claim 42, 43, or 44, and determining whether the compound alleviates the physical and behavioral abnormalities displayed by the transgenic, nonhuman mammal as a result of overactivity of a mammalian Edg7 receptor, the alleviation of the abnormality identifying the compound as an antagonist.

82. The method of claim 81, wherein the mammalian Edg7 receptor is a human Edg7 receptor.

83. An antagonist identified by the method of claim 81.

84. A pharmaceutical composition comprising an antagonist of claim 83 and a pharmaceutically acceptable carrier.

85. A method of treating an abnormality in a subject wherein the abnormality is alleviated by decreasing the activity of a mammalian Edg7 receptor which comprises administering to the subject an effective amount of the pharmaceutical composition of claim 84, thereby treating the abnormality.

86. A method for identifying an agonist capable of alleviating an abnormality in a subject wherein the abnormality is alleviated by increasing the activity of a mammalian Edg7 receptor comprising administering a compound to the transgenic, nonhuman mammal of claim 42, 43, or 44, and determining whether the compound alleviates the physical and behavioral abnormalities displayed by the transgenic, nonhuman mammal, the alleviation of the abnormality identifying the compound as an agonist.

87. The method of claim 86, wherein the mammalian Edg7 receptor is a human Edg7 receptor.

88. An agonist identified by the method of claim 86.

89. A pharmaceutical composition comprising an agonist identified by the method of claim 88 and a pharmaceutically acceptable carrier.

90. A method of treating an abnormality in a subject wherein the abnormality is alleviated by increasing the activity of a mammalian Edg7 receptor which comprises administering to the subject an effective amount of the pharmaceutical composition of claim 89, thereby treating the abnormality.

91. A method for diagnosing a predisposition to a disorder associated with the activity of a specific mammalian allele which comprises:
(a) obtaining DNA of subjects suffering from the disorder;
(b) performing a restriction digest of the DNA with,a panel of restriction enzymes;
(c) electrophoretically separating the resulting DNA fragments on a sizing gel;
(d) contacting the resulting gel with a nucleic acid probe capable of specifically hybridizing with a unique sequence included within the sequence of a nucleic acid molecule encoding a mammalian Edg7 receptor and labeled with a detectable marker;
(e) detecting labeled bands which have hybridized to the DNA encoding a mammalian Edg7 receptor of claim 1 labeled with a detectable marker to create a unique band pattern specific to the DNA of subjects suffering from the disorder;
(f) preparing DNA obtained for diagnosis by steps (a)-(e); and
(g) comparing the unique band pattern specific to the DNA of subjects suffering from the disorder from step (e) and the DNA obtained for diagnosis from step (f) to determine whether the patterns are the same or different and to diagnose thereby predisposition to the disorder if the patterns are the same.

92. The method of claim 91, wherein a disorder associated with the activity of a specific mammalian allele is diagnosed.

93. A method of preparing the purified mammalian Edg7 receptor of claim 9 which comprises:
(a) culturing cells which express the mammalian Edg7 receptor;
(b) recovering the mammalian Edg7 receptor from the cells; and
(c) purifying the mammalian Edg7 receptor so recovered.

94. A method of preparing the purified mammalian Edg7 receptor of claim 9 which comprises:
(a) inserting a nucleic acid encoding the mammalian Edg7 receptor into a suitable vector;
(b) introducing the resulting vector into a suitable host cell;
(c) placing the resulting cell in suitable condition permitting the production of the mammalian Edg7 receptor;
(d) recovering the mammalian Edg7 receptor produced by the resulting cell; and
(e) isolating and/or purifying the mammalian Edg7 receptor so recovered.

95. A process for determining whether a chemical compound is a mammalian Edg7 receptor agonist which comprises contacting cells transfected with and expressing DNA encoding the mammalian Edg7 receptor with the compound under conditions permitting the activation of the mammalian Edg7 receptor, and detecting an increase in mammalian Edg7 receptor activity, so as to thereby determine whether the compound is a mammalian Edg7 receptor agonist.

96. A process for determining whether a chemical compound is a mammalian Edg7 receptor antagonist which comprises contacting cells transfected with and expressing DNA encoding the mammalian Edg7 receptor with the compound in the presence of a known mammalian Edg7 receptor agonist, under conditions permitting the activation of the mammalian Edg7 receptor, and detecting a decrease in mammalian Edg7 receptor activity, so as to thereby determine whether the compound is a mammalian Edg7 receptor antagonist.

97. A process of claim 95 or 96, wherein the mammalian Edg7 receptor is a human Edg7 receptor.

98. A pharmaceutical composition which comprises an amount of a mammalian Edg7 receptor agonist determined by the process of claim 95 effective to increase activity of a mammalian Edg7 receptor and a pharmaceutically acceptable carrier.

99. A pharmaceutical composition of claim 98, wherein the mammalian Edg7 receptor agonist is not previously known.

100. A pharmaceutical composition which comprises an amount of a mammalian Edg7 receptor antagonist determined by the process of claim 96 effective to reduce activity of a mammalian Edg7 receptor and a pharmaceutically acceptable carrier.

101. A pharmaceutical composition of claim 100, wherein the mammalian Edg7 receptor antagonist is not previously known.

102. A process for determining whether a chemical compound specifically binds to and activates a mammalian Edg7 receptor, which comprises contacting cells producing a second messenger response and expressing on their cell surface the mammalian Edg7 receptor, wherein such cells do not normally express the mammalian Edg7 receptor, with the chemical compound under conditions suitable for activation of the mammalian Edg7 receptor, and measuring the second messenger response in the presence and in the absence of the chemical compound, a change in the second messenger response in the presence of the chemical compound indicating that the compound activates the mammalian Edg7 receptor.

103. The process of claim 102, wherein the second messenger response comprises chloride channel activation and the change in second messenger is an increase in the level of chloride current.

104. The process of claim 102, wherein the second messenger response comprises change in intracellular calcium levels and the change in second messenger is an increase in the measure of intracellular calcium.

105. The process of claim 102, wherein the second messenger response comprises release of inositol phosphate and the change in second messenger is an increase in the level of inositol phosphate.

106. A process for determining whether a chemical compound specifically binds to and inhibits activation of a mammalian Edg7 receptor, which comprises separately contacting cells producing a second messenger response and expressing on their cell surface the mammalian Edg7 receptor, wherein such cells do not normally express the mammalian Edg7 receptor, with both the chemical compound and a second chemical compound known to activate the mammalian Edg7 receptor, and with only the second chemical compound, under conditions suitable for activation of the mammalian Edg7 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the mammalian Edg7 receptor.

107. The process of claim 106, wherein the second messenger response comprises chloride channel activation and the change in second messenger response is a smaller increase in the level of chloride current in the presence of both the chemical compound and the second chemical compound than in the presence of only the second chemical compound.

108. The process of claim 106, wherein the second messenger response comprises change in intracellular calcium levels and the change in second messenger response is a smaller increase in the measure of intracellular calcium in the presence of both the chemical compound and the second chemical compound than in the presence of only the second chemical compound.

109. The process of claim 106, wherein the second messenger response comprises release of inositol phosphate and the change in second messenger response is a smaller increase in the level of inositol phosphate in the presence of both the chemical compound and the second chemical compound than in the presence of only the second chemical compound.

110. A process of any of claims 102, 103, 104, 105, 106, 107, 108, or 109, wherein the mammalian Edg7 receptor is a human Edg7 receptor.

111. The process of any of claims 102, 103, 104, 105, 106, 107, 108, or 109, wherein the cell is an insect cell.

112. The process of any of claims 102, 103, 104, 105, 106, 107, 108, or 109, wherein the cell is a mammalian cell.

113. The process of claim 112, wherein the mammalian cell is nonneuronal in origin.

114. The process of claim 113, wherein the nonneuronal cell is a COS-7 cell, CHO cell, 293 human embryonic kidney cell, NIH-3T3 cell or LM(tk-) cell.

115. The process of claim 102, 103, 104, 105, 106, 107, 108, or 109, wherein the compound is not previously known to bind to a mammalian Edg7 receptor.

116. A compound determined by the process of claim 115.

117. A pharmaceutical composition which comprises an amount of a mammalian Edg7 receptor agonist determined by the process of claim 102, 103, 104, or 105, effective to increase activity of a mammalian Edg7 receptor and a pharmaceutically acceptable carrier.

118. A pharmaceutical composition of claim 117, wherein the mammalian Edg7 receptor agonist is not previously known.

119. A pharmaceutical composition which comprises an amount of a mammalian Edg7 receptor antagonist determined by the process of claim 106, 107, 108, or 109, effective to reduce activity of a mammalian Edg7 receptor and a pharmaceutically acceptable carrier.

120. A pharmaceutical composition of claim 119, wherein the mammalian Edg7 receptor antagonist is not previously known.

121. A method of screening a plurality of chemical compounds not known to activate a mammalian Edg7 receptor to identify a compound which activates the mammalian Edg7 receptor which comprises:
(a) contacting cells transfected with and expressing the mammalian Edg7 receptor with the plurality of compounds not known to activate the mammalian Edg7 receptor, under conditions permitting activation of the mammalian Edg7 receptor;
(b) determining whether the activity of the mammalian Edg7 receptor is increased in the presence of the compounds; and if so
(c) separately determining whether the activation of the mammalian Edg7 receptor is increased by each compound included in the plurality of compounds, so as to thereby identify the compound which activates the mammalian Edg7 receptor.

122. A method of claim 121, wherein the mammalian Edg7 receptor is a human Edg7 receptor.

123. A method of screening a plurality of chemical compounds not known to inhibit the activation~of a mammalian Edg7 receptor to identify a compound which inhibits the activation of the mammalian Edg7 receptor, which comprises:
(a) contacting cells transfected with and expressing the mammalian Edg7 receptor with the plurality of compounds in the presence of a known mammalian Edg7 receptor agonist, under conditions permitting activation of the mammalian Edg7 receptor;
(b) determining whether the activation of the mammalian Edg7 receptor is reduced in the presence of the plurality of compounds, relative to the activation of the mammalian Edg7 receptor in the absence of the plurality of compounds; and if so
(c) separately determining the inhibition of activation of the mammalian Edg7 receptor for each compound included in the plurality of compounds, so as to thereby identify the compound which inhibits the activation of the mammalian Edg7 receptor.

124. A method of claim 123, wherein the mammalian Edg7 receptor is a human Edg7 receptor.

125. A method of any of claims 121, 122, 123, 124, wherein the cell is a mammalian cell.

126. A method of claim 125, wherein the mammalian cell is non-neuronal in origin.

127. The method of claim 126, wherein the non-neuronal cell is a COS-7 cell, a 293 human embryonic kidney cell, a LM(tk-) cell or an NIH-3T3 cell.

128. A pharmaceutical composition comprising a compound identified by the method of claim 121 or 122 effective to increase mammalian Edg7 receptor activity and a pharmaceutically acceptable carrier.

129. A pharmaceutical composition comprising a compound identified by the method of claim 123 or 124 effective to decrease mammalian Edg7 receptor activity and a pharmaceutically acceptable carrier.

130. A method of treating an abnormality in a subject wherein the abnormality is alleviated by increasing the activity of a mammalian Edg7 receptor which comprises administering to the subject an amount of a compound which is a mammalian Edg7 receptor agonist effective to treat the abnormality.

131. A method of treating an abnormality in a subject wherein the abnormality is alleviated by decreasing the activity of a mammalian Edg7 receptor which comprises administering to the subject an amount of a compound which is a mammalian Edg7 receptor antagonist effective to treat the abnormality.

132. A process for making a composition of matter which specifically binds to a mammalian Edg7 receptor which comprises identifying a chemical compound using the process of any of claims 48, 49, 62, 63, 71, or 72 and then synthesizing the chemical compound or a novel structural and functional analog or homolog thereof.

133. The process of claims 132, wherein the mammalian Edg7 receptor is a human Edg7 receptor.

134. A process for making a composition of matter which specifically binds to a mammalian Edg7 receptor which comprises identifying a chemical compound using the process of any of claims 95, 102, or 121 and then synthesizing the chemical compound or a novel structural and functional analog or homolog thereof.

135. The process of claim 134, wherein the mammalian Edg7 receptor is a human Edg7 receptor.

136. A process for making a composition of matter which specifically binds to a mammalian Edg7 receptor which comprises identifying a chemical compound using the process of any of claims 96, 106, or 123 and then synthesizing the chemical compound or a novel structural and functional analog or homolog thereof.

137. The process of claim 136, wherein the mammalian Edg7 receptor is a human Edg7 receptor.

138. A process for preparing a pharmaceutical composition which comprises admixing a pharmaceutically acceptable carrier and a pharmaceutically acceptable amount of a chemical compound identified by the process of any of claims 48, 49, 62, 63, 71, or 72 or a novel structural and functional analog or homolog thereof.

139. The process of claim 138, wherein the mammalian Edg7 receptor is a human Edg7 receptor.

140. A process for preparing a pharmaceutical composition which comprises admixing a pharmaceutically acceptable carrier and a pharmaceutically acceptable amount of a chemical compound identified by the process of any of claims 95, 102, or 121 or a novel structural and functional analog or homolog thereof.

141. The process of claim 140, wherein the mammalian Edg7 receptor is a human Edg7 receptor.

142. A process for preparing a pharmaceutical composition which comprises admixing a pharmaceutically acceptable carrier and a pharmaceutically acceptable amount of a chemical compound identified by the process of any of claims 96, 106, or 123 or a novel structural and functional analog or homolog thereof.

143. The process of claim 142, wherein the mammalian Edg7 receptor is a human Edg7 receptor.
